(19) United States
(12) Patent Application Publication
(10) Pub. No.: US 2026/0167740 A1
(43) Pub. Date: Jun. 18, 2026

(54) MEANS AND METHODS FOR TREATING CASTRATION-RESISTANT PROSTATE CANCER

(71) Applicant: Merus N.V., Utrecht (NL)

(72) Inventors: Ernesto Isaac WASSERMAN, Utrecht (NL); Jeroen Jilles LAMMERTS VAN BUEREN, Utrecht (NL); Farida Anastasia Leila MURAT, Utrecht (NL)

(21) Appl. No.: 19/101,181

(22) PCT Filed: Aug. 4, 2023
(86) PCT No.: PCT/NL2023/050416

§ 371 Date: Feb. 4, 2025

(30) Foreign Application Priority Data
Aug. 5, 2022 (NL) 2032695

(51) Int. Cl. C07K 16/32; A61K 31/4166; A61K 31/58; A61K 39/00; A61K 39/395; A61P 35/00

(52) U.S. Cl. CPC C07K 16/32; A61K 31/4166; A61K 31/58; A61K 39/3955; A61P 35/00; A61K 2039/505; A61K 2039/545; C07K 2317/31

(57) ABSTRACT

The invention relates to the field of therapeutic antibodies for the treatment of a subject with castration-resistant prostate cancer. More in particular it relates to treating castration-resistant prostate cancer using a bispecific antibody that comprises an antigen binding site that can bind an extracellular part of ERBB2 and an antigen binding site that can bind an extracellular part of ERBB3. Also, it relates to treating castration-resistant prostate cancer using an antibody that comprises an antigen binding site that can bind an extracellular part of ERBB3. The invention also relates to treating castration-resistant prostate cancer using a combination of one of said antibodies and an androgen receptor axis-targeting agent.

### BACKGROUND OF THE INVENTION

The invention relates to the field of antibodies. In particular it relates to the field of therapeutic (human) antibodies for the treatment of castration-resistant prostate cancer. More in particular it relates to antibodies that bind ERBB3, and to antibodies that bind ERBB2 and ERBB3 and their use in the treatment of cancer in combination treatment, or combination therapy, with an androgen receptor axis-targeting agent.

In 2020, prostate cancer was the second most common malignancy in males globally and the fifth most common cause of cancer deaths (Sung et al. “Global Cancer Statistics 2020: GLOBOCAN Estimates of Incidence and Mortality Worldwide for 36 Cancers in 185 Countries.” CA Cancer J Clin 71(3): 209-249, 2021). In 2021, the number of cases of advanced prostate cancer in the United States was estimated to be approximately 250,000 (Siegel et al, “Cancer Statistics, 2021.” CA Cancer J Clin 71(1): 7-33, 2021), which includes a subpopulation of 30,000 men with castration-resistant prostate cancer (CRPC) (Scher et al. Prostate Cancer Clinical Trials Working (2016). “Trial Design and Objectives for Castration-Resistant Prostate Cancer: Updated Recommendations From the Prostate Cancer Clinical Trials Working Group 3.” J Clin Oncol 34(12): 1402-1418, 2015). In patients with locally advanced, recurrent or metastatic tumors, goals of present therapy include to prolong survival and the progression-free interval, while maintaining a good quality of life.

Since the 1940s, the primary therapy for men with metastatic prostate cancer has been Androgen Deprivation Therapy (ADT) alone, to suppress the production of testosterone, either using surgical or chemical castration. Chemotherapy is typically initiated only after the patient no longer responds to ADT alone, which is when disease is considered to be castration resistant.

The androgen receptor (AR) is a lineage survival factor for luminal cancer cells in prostate tumor that plays a role in cancer. ADT is currently still the main treatment modality for advanced/metastatic disease, targeting AR signaling. Next-generation AR signaling inhibitors approved for first-line treatment include abiraterone and enzalutamide (Swami et al. 2020, “Advanced Prostate Cancer: Treatment Advances and Future Directions.” Trends Cancer 6(8): 702-715). These inhibitors enable stronger blockade of the androgen receptor (AR) axis and longer survival of men afflicted with CRPC. However, the extent of the improved survival remains insufficient and the majority of patients eventually develop resistance to these novel agents. The mechanisms of resistance to next-generation hormonal agents are complex and may be dependent or independent of AR signaling, and the AR axis may remain an important driver in advanced/metastatic CRPC after progression (Verma et al. 2020, Resistance to second generation antiandrogens in prostate cancer: pathways and mechanisms.” Cancer Drug Resist 3(4): 742-761).

While preclinical studies have implicated the ERBB signaling pathway in the progression of CRPC (Craft et al., 1999. A mechanism for hormone independent prostate cancer through modulation of androgen receptor signaling by the HER-2/neu tyrosine kinase. Nat Med 5(3): 280-285), successful targeting of this pathway is yet to materialize in the clinical setting, with failure to demonstrate antitumor activity of ERBB2-targeting agents, including trastuzumab and afatinib (Ziada et al. 2004, The use of trastuzumab in the treatment of hormone refractory prostate cancer; phase II trial. Prostate 60(4): 332-337. Molife et al. 2014, Randomized Phase II trial of nintedanib, afatinib and sequential combination in castration-resistant prostate cancer. Future Oncol 10(2): 219-231.).

More than 30,000 patients with metastatic CRPC (mCRPC) are currently receiving first or second line systemic therapy but invariably progress, warranting subsequent lines of therapy. There is thus a significant unmet medical need for additional treatment options in patients who progress after treatment with AR signaling inhibitors. Besides, a growing number of patients with non-metastatic prostate cancer currently treated with AR signaling inhibitors will progress, expanding the pool of CRPC post-AR signaling inhibitors patients.

Accordingly, there is a need in the field for treatment of castration-resistant prostate cancer.

### SUMMARY OF THE INVENTION

The present disclosure provides a bispecific antibody that comprises an antigen binding site that can bind an extracellular part of ERBB2 and an antigen binding site that can bind an extracellular part of ERBB3 for use in a method of treatment of a castration-resistant prostate cancer in a subject. Said method comprises administering an effective amount of a bispecific antibody that comprises an antigen binding site that can bind an extracellular part of ERBB2 and an antigen binding site that can bind an extracellular part of ERBB3 to the subject. In certain aspects, said method of treatment further comprises the use of an androgen receptor axis-targeting agent.

The present disclosure provides a method of treatment of castration-resistant prostate cancer in a subject, the method comprising administering to the subject a therapeutically effective amount of a bispecific antibody that comprises an antigen binding site that can bind an extracellular part of ERBB2 and an antigen binding site that can bind an extracellular part of ERBB3. In certain aspects, the method comprises selecting a subject having castration-resistant prostate cancer, or a subject being suspected of having castration-resistant prostate cancer, prior to being treated for castration-resistant prostate cancer.

The present disclosure provides an antibody that comprises an antigen binding site that can bind an extracellular part of ERBB3 for use in a method of treatment of a castration-resistant prostate cancer in a subject. Said method comprises administering an effective amount of an antibody that comprises an antigen binding site that can bind an extracellular part of ERBB3 to the subject. In certain aspects, said method of treatment further comprises the use of an androgen receptor axis-targeting agent. In certain aspects, the antibody is a monospecific antibody.

The present disclosure provides a method of treatment of castration-resistant prostate cancer in a subject, the method comprising administering to the subject a therapeutically effective amount of a bispecific antibody that comprises an antigen binding site that can bind an extracellular part of ERBB3. In certain aspects, the method comprises selecting a subject having castration-resistant prostate cancer, or a subject being suspected of having castration-resistant prostate cancer, prior to being treated for castration-resistant prostate cancer. In certain aspects, the antibody is a monospecific antibody.

In certain aspects, said method of treatment further comprises the screening of a patient being suspected of having castration-resistant prostate cancer according to certain inclusion and exclusion criteria.

In certain aspects, said method of treatment further comprises the administration of an androgen receptor axis-targeting agent to the subject.

The present disclosure provides a use of a bispecific antibody that comprises an antigen binding site that can bind an extracellular part of ERBB2 and an antigen binding site that can bind an extracellular part of ERBB3 for the manufacture of a medicament for the treatment of castration-resistant prostate cancer in a subject. In certain aspects, said treatment or use further comprises the use of an androgen receptor axis-targeting agent.

In certain aspects, the castration-resistant prostate cancer has progressed after prior cancer treatment with an androgen receptor axis-targeting agent. In certain aspects, the castration-resistant prostate cancer has progressed after prior treatment with an androgen receptor antagonist, such as a second generation androgen receptor antagonist, or has progressed after prior treatment with an androgen synthesis inhibitor, such as abiraterone acetate. In certain aspects, the castration-resistant prostate cancer has progressed after prior treatment with enzalutamide.

In certain aspects, methods of treatment which include the use or administration of the bispecific antibody of the present disclosure, further comprise the use or administration of an androgen receptor axis-targeting agent. In certain aspects, said bispecific antibody and said androgen receptor axis-targeting agent are administered within the same time period. Said time period typically comprises a cycle of 28 days during which the bispecific antibody is administered twice, typically on day 1 and day 15 of said cycle (i.e. Q2W), and said androgen receptor axis-targeting agent is administered on each day of the cycle (i.e. QD). Administration of said bispecific antibody and/or said androgen receptor axis-targeting agent is continued until a decision is made to discontinue treatment but may proceed for as long as deemed clinically relevant or for as long as a clinically relevant effect is observed.

Thus, in certain aspects, the cancer has progressed after prior treatment with an androgen receptor axis-targeting agent. In certain aspects, if the cancer has progressed after prior treatment with an androgen receptor antagonist, the use of the bispecific antibody in the method of treatment according to the present disclosure further comprises the use of an androgen receptor antagonist. In certain aspects, if the cancer has progressed after prior treatment with an androgen receptor antagonist, the method of treatment according to the present disclosure with said bispecific antibody further comprises administration of an androgen receptor antagonist. A combined use is thus foreseen wherein the bispecific antibody that comprises an antigen binding site that can bind an extracellular part of ERBB2 and an antigen binding site that can bind an extracellular part of ERBB3, is used during the same treatment period with an androgen receptor antagonist.

The disclosure thus provides in certain aspects a combination therapy comprising a first container comprising an antibody as described herein together with a second container comprising an androgen receptor axis-targeting agent. The two components may be formulated as separate pharmaceutical compositions (e.g., as a kit-of-parts) and may be administered simultaneously, separately or sequentially in any order. As a skilled person will appreciate, the two components may be provided at different times during the same treatment cycle. In certain aspects, a treatment cycle is made up of 28 days. In certain aspects, a bispecific antibody of the disclosure is administered twice during a treatment cycle (e.g., Q2W) and an androgen receptor axis-targeting agent is administered daily during a treatment cycle.

In certain aspects, the androgen receptor antagonist used in the prior cancer treatment is the same androgen receptor antagonist as used in the method of treatment in combination with the bispecific antibody of the present disclosure. In certain aspects, the androgen receptor antagonist used in the prior cancer treatment and as used in combination with the bispecific antibody of the present disclosure are both enzalutamide. Thus, in these aspects, the present disclosure provides a combination treatment, or combination therapy, wherein the bispecific antibody that comprises an antigen binding site that can bind an extracellular part of ERBB2 and an antigen binding site that can bind an extracellular part of ERBB3, such as zenocutuzumab, is used with an androgen receptor antagonist, such as enzalutamide.

In certain aspects, enzalutamide is administered at a daily dose of 160 mg.

In certain aspects, the cancer has progressed after prior treatment with an androgen synthesis inhibitor. In certain aspects, if the cancer has progressed after prior treatment with an androgen synthesis inhibitor, the use of the bispecific antibody in the method of treatment according to the present disclosure further comprises the use of an androgen synthesis inhibitor. In certain aspects, if the cancer has progressed after prior treatment with an androgen synthesis inhibitor, the method of treatment according to the present disclosure with the bispecific antibody further comprises the administration of an androgen synthesis inhibitor. A combined use is thus foreseen wherein the bispecific antibody that comprises an antigen binding site that can bind an extracellular part of ERBB2 and an antigen binding site that can bind an extracellular part of ERBB3, is used during the same treatment period with an androgen synthesis inhibitor.

In certain aspects, the androgen synthesis inhibitor used in the prior cancer treatment is the same androgen synthesis inhibitor as used in the method of treatment in combination with the bispecific antibody of the present disclosure. In certain aspects, the androgen synthesis inhibitor used in the prior cancer treatment and as used in combination with the bispecific antibody of the present disclosure are both abiraterone acetate, such as ZYTIGA®. Thus, in this aspect, the present disclosure provides a combination treatment, or combination therapy, wherein the bispecific antibody that comprises an antigen binding site that can bind an extracellular part of ERBB2 and an antigen binding site that can bind an extracellular part of ERBB3, such as zenocutuzumab, is used during the same treatment period with an androgen synthesis inhibitor, such as abiraterone acetate, for instance ZYTIGA®.

In certain aspects, abiraterone acetate, such as ZYTIGA®, is administered at a daily dose of 1000 mg. In certain aspects, abiraterone acetate is used in combination with an oral administration of prednisone at 5 mg twice daily.

In certain aspects, the bispecific antibody of the present disclosure is administered or used in an amount of 750 mg once every two weeks. In certain aspects, the bispecific antibody is zenocutuzumab.

In certain aspects, an antibody of the present disclosure comprises an antigen binding site that can bind an extracellular part of ERBB3 which blocks both ERBB3 and its ligand heregulin. In certain aspects, said antibody binds domain III of ERBB3. In certain aspects, said antibody also comprises an antigen binding site that can bind an extracellular part of ERBB2. In certain aspects, said antibody comprises an antigen binding site that can bind an extracellular part of ERBB2 and an antigen binding site that can bind an extracellular part of ERBB3. In certain aspects, said antibody is a bispecific antibody. In certain aspects, the bispecific antibody of the present disclosure comprises a first antigen-binding site that can bind an extracellular part of ERBB2 and a second antigen-binding site that can bind an extracellular part of ERBB3. In certain aspects, said bispecific antibody has a first antigen-binding site that can bind domain I of ERBB2 and a second antigen-binding site that can bind domain III of ERBB3. In certain aspects, the affinity of the first antigen-binding site for ERBB2 is lower than the affinity of the second antigen-binding site for ERBB3. In certain aspects, said antibody is or comprises zenocutuzumab.

In certain aspects, the present disclosure provides methods of treatments, as disclosed herein, wherein an antibody that comprises an antigen binding site that can bind an extracellular part of ERBB3 is used. In certain aspects, the antibody is a monospecific antibody.

In certain aspects, the present disclosure provides a method of treatment of a subject having a castration-resistant prostate cancer, the method comprising administering to the subject an antibody that comprises an antigen binding site that can bind an extracellular part of ERBB3. In certain aspects, the antibody comprises an antigen-binding site that can bind domain III of ERBB3.

In certain aspects, the present disclosure provides a use of an antibody that comprises an antigen binding site that can bind an extracellular part of ERBB3 for the manufacture of a medicament for the treatment castration-resistant prostate cancer in a subject. In certain aspects, said the antibody comprises an antigen-binding site that can bind domain III of ERBB3.

In certain aspects, an antibody of the present disclosure, including but not limited to a bispecific antibody, comprises an antigen binding site that can bind an extracellular part of ERBB3 which blocks both ERBB3 and its ligand heregulin. In certain aspects, said antibody also comprises an antigen binding site that can bind an extracellular part of ERBB3. In certain aspects, said antibody comprises an antigen binding site that can bind an extracellular part of ERBB2 and an antigen binding site that can bind an extracellular part of ERBB3. In certain aspects, said antibody is or comprises zenocutuzumab.

Also provided is a method of selecting a subject having castration-resistant prostate cancer for treatment with a bispecific antibody that comprises an antigen binding site that can bind an extracellular part of ERBB2 and an antigen binding site that can bind an extracellular part of ERBB3, or for treatment with an antibody that comprises an antigen binding site that can bind an extracellular part of ERBB3, the method comprising: a) determining PTEN status in a sample obtained from the subject; and b) selecting the subject for said treatment if the sample does not exhibit PTEN loss.

Also provided is a method of establishing whether a subject having castration-resistant prostate cancer is likely to respond to treatment with a bispecific antibody that comprises an antigen binding site that can bind an extracellular part of ERBB2 and an antigen binding site that can bind an extracellular part of ERBB3, or to respond to treatment with an antibody that comprises an antigen binding site that can bind an extracellular part of ERBB3, the method comprising: a) determining PTEN status in a sample obtained from the subject; and b) selecting a sample for not exhibiting PTEN loss, thereby establishing that the subject from which, or from whom, the sample is derived is likely to respond to said treatment.

Also provided is a method of classifying a subject having castration-resistant prostate cancer on the basis of PTEN status prior to treatment with a bispecific antibody that comprises an antigen binding site that can bind an extracellular part of ERBB2 and an antigen binding site that can bind an extracellular part of ERBB3, or prior to treatment with an antibody that comprises an antigen binding site that can bind an extracellular part of ERBB3, the method comprising: a) determining PTEN status in a sample obtained from the subject; and b) classifying the subject from which, or from whom, the sample was obtained as eligible for said treatment if the sample does not exhibit PTEN loss.

Also provided is a method of selecting a subject having castration-resistant prostate cancer for treatment with a bispecific antibody that comprises an antigen binding site that can bind an extracellular part of ERBB2 and an antigen binding site that can bind an extracellular part of ERBB3, or for treatment with an antibody that comprises an antigen binding site that can bind an extracellular part of ERBB3, the method comprising: a) determining PTEN status in a sample obtained from the subject; and b) selecting the subject for said treatment if the sample exhibits PTEN loss.

Also provided is a method of establishing whether a subject having castration-resistant prostate cancer is likely to respond to treatment with a bispecific antibody that comprises an antigen binding site that can bind an extracellular part of ERBB2 and an antigen binding site that can bind an extracellular part of ERBB3, or to respond to treatment with an antibody that comprises an antigen binding site that can bind an extracellular part of ERBB3, the method comprising: a) determining PTEN status in a sample obtained from the subject; and b) selecting a sample for exhibiting PTEN loss, thereby establishing that the subject from which, or from whom, the sample is derived is likely to respond to said treatment.

Also provided is a method of classifying a subject having castration-resistant prostate cancer on the basis of PTEN status prior to treatment with a bispecific antibody that comprises an antigen binding site that can bind an extracellular part of ERBB2 and an antigen binding site that can bind an extracellular part of ERBB3, or prior to treatment with an antibody that comprises an antigen binding site that can bind an extracellular part of ERBB3, the method comprising: a) determining PTEN status in a sample obtained from the subject; and b) classifying the subject from which, or from whom, the sample was obtained as eligible for said treatment if the sample exhibits PTEN loss.

In certain aspects, the subject is a human subject. The human subject has castration-resistant prostate cancer, or is at risk of suffering therefrom. In a certain aspect, the subject has metastatic castration-resistant prostate cancer or is at risk of developing metastatic castration-resistant prostate cancer. As such, said subject is in need of treatment of castration-resistant prostate cancer or metastatic castration-resistant prostate cancer.

### BRIEF DESCRIPTION OF THE DRAWINGS

FIG. 1 lists the amino acid sequence of a) a common light chain variable region amino acid sequence (VL+CL sequences); b) common light chain variable region DNA sequence and its translation (IGKV1-39/jk1). c) Common light chain constant region DNA sequence and translation. d) IGKV1-39/jk5 common light chain variable region translation. e) V-region IGKV1-39A; f) common light chain CDR1, CDR2 and CDR3 sequences, all according to IMGT.

FIG. 2 lists the IgG heavy chains sequences for the generation of bispecific molecules. a) CH1 region, b) hinge region, c) CH2 region, d) CH3 domain containing variations L351K and T366K (KK), e) CH3 domain containing variations L351D and L368E (DE).

FIG. 3 lists the nucleic acid and amino acid sequences of the heavy chain of variable regions. CDR1, CDR2 and CDR3 sequences, according to the Kabat numbering system, of the respective heavy chain variable regions are also provided.

### DETAILED DESCRIPTION OF THE INVENTION

A proportion of CRPC patients present with primary resistance, and ultimately almost all patients develop resistance. The development of CRPC resistance is thought to be a subclonal phenomenon, with a proportion of the tumoral cells remaining sensitive to androgen-deprivation therapy despite the emergence of resistant clones. Without being bound to theory, it is the inventors insight that preventing paracrine activation of NRG1/ERBB3 signaling to blunt a driver of anti-androgen resistance in CRPC is an option for treatment with an antibody that comprises an antigen binding site that can bind an extracellular part of ERBB3 at domain III or an antibody that comprises an antigen binding site that can bind an extracellular part of ERBB3 at domain III and an antigen binding site that can bind an extracellular part of ERBB2 at domain I, as disclosed herein for use in a method of treatment of a castration-resistant prostate cancer in a subject.

The ERBB family of tyrosine kinase transmembrane receptors are also referred to as the human epidermal growth factor (EGF) receptor family (HER). The family has four members: ERBB (Erythroblastoma)-1, ERBB2, ERBB3 and ERBB4. The receptors (reviewed in Yarden and Pines 2012) are widely expressed on epithelial cells.

Upregulation of HER receptors or their ligands, such as, neuregulin (NRG) (also known heregulin (HRG)) or epidermal growth factor (EGF), is a frequent event in human cancer (Wilson, Timothy R et al. Nature, vol. 487, 7408 (2012): 505-9). Overexpression of ERBB1 and ERBB2 in particular occurs in epithelial tumors and is associated with tumor invasion, metastasis, resistance to chemotherapy, and poor prognosis (Zhang, Hongtao et al. The Journal of clinical investigation, vol. 117, 8 (2007): 2051-8). In the normal breast, ERBB3 has been shown to be important in the growth and differentiation of luminal epithelium. For instance, loss/inhibition of ERBB3 results in selective expansion of the basal over the luminal epithelium (Balko, Justin M et al. Proceedings of the National Academy of Sciences, vol. 109, 1 (2012): 221-6). Binding of ligand to the extracellular domain of the RTKs induces receptor dimerization, both between the same (homodimerization) and different (heterodimerization) receptor subtypes. Dimerization can activate the intracellular tyrosine kinase domains, which undergo autophosphorylation and, in turn, can activate a number of downstream pro-proliferative signaling pathways, including those mediated by mitogen-activated protein kinases (MAPK) and the prosurvival pathway Akt (reviewed in Yarden, Yosef, and Gur Pines. Nature reviews. Cancer, vol. 12, 8 553-63). No specific endogenous ligand has been identified for ERBB2, which is therefore assumed to normally signal through heterodimerization (Sergina, Natalia V et al. Nature, vol. 445, 7126 (2007): 437-41). ERBB3 can be activated by engagement of its ligands. These ligands include but are not limited to neuregulin (NRG) (also known as heregulin (HRG)).

ERBB1 is known under various synonyms, the most common of which is EGFR. EGFR has an extracellular domain (ECD) that is composed of four sub-domains, two of which are involved in ligand binding and two of which are involved in homodimerization and heterodimerization. EGFR integrates extracellular signals from a variety of ligands to yield diverse intracellular responses. The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This receptor tyrosine kinase (RTK) has been extensively used as target for cancer therapy. Both small-molecule inhibitors targeting the RTK and monoclonal antibodies (mAbs) (monospecific bivalent) directed to the extracellular ligand-binding domains have been developed and have shown hitherto several clinical successes. The database accession number for the human EGFR protein and the gene encoding it is (GenBank NM_005228.3). This accession number is primarily given to provide a further method of identification of EGFR protein as a target, the actual sequence of the EGFR protein bound by an antibody may vary, for instance because of a mutation in the encoding gene such as those occurring in some cancers or the like.

The term ‘ERBB2’ as used herein refers to the protein that in humans is encoded by the ERBB2 gene. Alternative names for the gene or protein include CD340; HER2; HER-2/neu; MLN 19; NEU; NGL; TKR1. The ERBB2 gene is frequently called HER2 (from human epidermal growth factor receptor 2). Where reference is made herein to ERBB2, the reference refers to human ERBB2. An antibody comprising an antigen-binding site that binds ERBB2, binds human ERBB2. The ERBB2 antigen-binding site may, due to sequence and tertiary structure similarity between human and other mammalian orthologs, also bind such an ortholog but not necessarily so. Database accession numbers for the human ERBB2 protein and the gene encoding it are (NP_001005862.1, NP_004439.2 NC_000017.10 NT_010783.15 NC_018928.2). The accession numbers are primarily given to provide a further method of identification of ERBB2 as a target, the actual sequence of the ERBB2 protein bound the antibody may vary, for instance because of a mutation in the encoding gene such as those occurring in some cancers or the like. The ERBB2 antigen binding site binds ERBB2 and a variety of variants thereof, such as those expressed by some ERBB2 positive tumor cells. The antigen-binding site that binds ERBB2 preferably binds domain I of ERBB2.

The term ‘ERBB3’ as used herein refers to the protein that in humans is encoded by the ERBB3 gene. Alternative names for the gene or protein are HER3; LCCS2; MDA-BF-1; c-ERBB3; c-ERBB3; ERBB3-S; p180-ERBB3; p45-sERBB3; and p85-sERBB3. Where reference is made herein to ERBB3, the reference refers to human ERBB3. An antibody comprising an antigen-binding site that binds ERBB3, binds human ERBB3. The ERBB3 antigen-binding site may, due to sequence and tertiary structure similarity between human and other mammalian orthologs, also bind such an ortholog but not necessarily so. Database accession numbers for the human ERBB3 protein of the present disclosure and the gene encoding it are NP_001973.2 and NC_000012.11 which contains the genomic location of the ERBB3 gene on chromosome 12 (56473892 to 56497289). The accession numbers are primarily given to provide a further method of identification of ERBB3 as a target, the actual sequence of the ERBB3 protein bound by an antibody may vary, for instance because of a mutation in the encoding gene such as those occurring in some cancers or the like. The ERBB3 antigen binding site binds ERBB3 and a variety of variants thereof, such as those expressed by some ERBB3 positive tumor cells. The antigen-binding site that binds ERBB3 preferably binds domain III of ERBB3.

When reference is made to ERBB1, ERBB2 or ERBB3 or an alternative name for the same, the reference is to human ERBB1, ERBB2 or ERBB3. Antibodies as referred to herein bind to ERBB1, ERBB2 or ERBB3 and many mutated ERBB1, ERBB2 or ERBB3 proteins as can be found in cancers.

The present disclosure provides a bispecific antibody that comprises an antigen binding site that binds or can bind an extracellular part of ERBB2 and an antigen binding site that binds or can bind an extracellular part of ERBB3 for use in a method of treatment of a castration-resistant prostate cancer in a subject. Said method comprises administering of an effective amount of a bispecific antibody that comprises an antigen binding site that binds or can bind an extracellular part of ERBB2 and an antigen binding site that binds or can bind an extracellular part of ERBB3 to the subject. In certain aspects, said method of treatment further comprises the use of an androgen receptor axis-targeting agent.

The present disclosure provides a method of treatment of castration-resistant prostate cancer in a subject, the method comprising administering to the subject an effective amount of a bispecific antibody that comprises an antigen binding site that binds or can bind an extracellular part of ERBB2 and an antigen binding site that binds or can bind an extracellular part of ERBB3. In certain aspects, the method comprises selecting a subject having castration-resistant prostate cancer, or a subject being suspected of having castration-resistant prostate cancer, prior to being treated for castration-resistant prostate cancer. In certain aspects, said method of treatment further comprises the administration of an androgen receptor axis-targeting agent to the subject.

The present disclosure provides a use of a bispecific antibody that comprises an antigen binding site that binds or can bind an extracellular part of ERBB2 and an antigen binding site that binds or can bind an extracellular part of ERBB3 for the manufacture of a medicament for the treatment of castration-resistant prostate cancer in a subject. In certain aspects, said treatment or use further comprises the use of an androgen receptor axis-targeting agent.

Endocrine and metabolic consequences represent the primary adverse effects associated with the safety profile of hormone therapies directed against prostate cancer. For instance, treatment with abiraterone acetate induces mineralocorticoid excess, hypokalemia, hypertension, elevated liver function tests, insulin resistance, and hyperglycemia. Enzalutamide is known to induce or worsen hypertension and is associated with increased falls and fractures in elderly patients. As such, limited overlapping toxicity with zenocutuzumab is anticipated with the safety profiles associated with abiraterone acetate and enzalutamide and the very well tolerated safety profile of zenocutuzumab renders it a particular good candidate for combination therapy in this advanced and pretreated often elderly patient population. Continued blockade of CRPC with an AR signaling inhibitor while simultaneously blocking the HER3/NRG1-resistance signaling pathway manages patients after failure on a next-generation AR agent. Continued treatment of CRPC with an androgen targeting agent while simultaneously using an antibody that comprises an antigen binding site that can bind an extracellular part of ERBB3 at domain III or an antibody that comprises an antigen binding site that can bind an extracellular part of ERBB3 at domain III and an antigen binding site that can bind an extracellular part of ERBB2 at domain I, is disclosed herein for use in a method of treatment of a castration-resistant prostate cancer in a subject.

In certain aspects, said castration-resistant prostate cancer has progressed after having received prior cancer treatment. Such prior treatment, also known as anti-cancer therapy or treatment, in some aspects includes an androgen receptor axis-targeting agent. In certain aspects, the cancer has progressed after prior treatment with an androgen receptor antagonist, such as a second generation androgen receptor antagonist. In certain aspects, the cancer has progressed after prior treatment with a first generation androgen receptor antagonist, such as flutamide, bicalutamide or nilutamide, or a second generation androgen receptor antagonist such as apalutamide, darolutamide or enzalutamide or an androgen receptor antagonist known to persons of ordinary skill in the art, such as proxalutamide, BMS-641988, TQB3720, SHR3680 or TRC-253. In certain aspects, the cancer has progressed after prior treatment with enzalutamide.

In certain aspects, castration-resistant prostate cancer is metastatic castration-resistant prostate cancer (mCRPC).

In certain aspects, methods of treatment which include the use or administration of the bispecific antibody of the present disclosure, further comprise the use or administration of an androgen receptor axis-targeting agent.

Thus, in certain aspects, the cancer has progressed after prior treatment with an androgen receptor axis-targeting agent. In certain aspects, if the cancer has progressed after prior treatment with an androgen receptor antagonist, the use of the bispecific antibody in the method of treatment according to the present disclosure further comprises the use of an androgen receptor antagonist. In certain aspects, if the cancer has progressed after prior treatment with an androgen receptor antagonist, the method of treatment according to the present disclosure with said bispecific antibody further comprises administration of an androgen receptor antagonist. A combined use is thus foreseen wherein the bispecific antibody that comprises an antigen binding site that can bind an extracellular part of ERBB2 and an antigen binding site that can bind an extracellular part of ERBB3, is used during the same treatment period with an androgen receptor antagonist. Thus, in certain aspects, said cancer has progressed after prior treatment with an androgen receptor antagonist and the method of treatment with the antibody further comprises the use of an androgen receptor antagonist.

In certain aspects, the androgen receptor antagonist used in the prior cancer treatment is the same androgen receptor antagonist as used in the method of treatment in combination with the bispecific antibody of the present disclosure. In certain aspects, the androgen receptor antagonist used in the prior cancer treatment and as used in combination with the bispecific antibody of the present disclosure are both apalutamide, darolutamide or enzalutamide. In certain aspects, the androgen receptor antagonist used in the prior cancer treatment and as used in combination with the antibody of the present disclosure are both enzalutamide. Thus, in this aspect, the present disclosure provides a combination treatment, or combination therapy, wherein the bispecific antibody that comprises an antigen binding site that can bind an extracellular part of ERBB2 and an antigen binding site that can bind an extracellular part of ERBB3, such as zenocutuzumab, is used with an androgen receptor antagonist, such as enzalutamide.

In certain aspects, an antibody of the present disclosure is combined with an androgen receptor antagonist which includes flutamide, bicalutamide, nilutamide, a second generation androgen receptor antagonist, such as apalutamide, darolutamide or enzalutamide, or an androgen receptor antagonist that known to persons of ordinary skill in the art, such as proxalutamide, BMS-641988, TQB3720, SHR3680 or TRC-253.

In certain aspects, enzalutamide is administered at a daily dose of 160 mg. In certain aspects, enzalutamide is administered at a daily dose of 160 mg and the bispecific antibody of the present disclosure is administered in an amount of 750 mg once every two weeks. In certain aspects, said bispecific antibody is zenocutuzumab. Thus, treatment of said subject comprises both administration of enzalutamide and zenocutuzumab. In certain aspects, enzalutamide used in the prior cancer treatment as well as further included in the present method of treatment is administered at a daily dose of 160 mg.

In certain aspects, the cancer has progressed after prior treatment with an androgen synthesis inhibitor. In certain aspects, if the cancer has progressed after prior treatment with an androgen synthesis inhibitor, the use of the bispecific antibody in the method of treatment according to the present disclosure further comprises the use of an androgen synthesis inhibitor. In certain aspects, if the cancer has progressed after prior treatment with an androgen synthesis inhibitor, the method of treatment according to the present disclosure with the bispecific antibody further comprises the administration of an androgen synthesis inhibitor. A combined use is thus foreseen wherein the bispecific antibody that comprises an antigen binding site that can bind an extracellular part of ERBB2 and an antigen binding site that can bind an extracellular part of ERBB3, is used during the same treatment period with an androgen synthesis inhibitor. Thus, in certain aspects, said cancer has progressed after prior treatment with an androgen synthesis inhibitor and the method of treatment with the antibody further comprises the use of an androgen synthesis inhibitor.

In certain aspects, the androgen synthesis inhibitor used in the prior cancer treatment is the same androgen synthesis inhibitor as used in the method of treatment in combination with the bispecific antibody of the present disclosure. In certain aspects, the androgen synthesis inhibitor used in the prior cancer treatment and as used in combination with the bispecific antibody of the present disclosure are both abiraterone acetate, such as ZYTIGA®. Thus, in this aspect, the present disclosure provides a combination treatment, or combination therapy, wherein the bispecific antibody that comprises an antigen binding site that can bind an extracellular part of ERBB2 and an antigen binding site that can bind an extracellular part of ERBB3, such as zenocutuzumab, is used during the same treatment period with an androgen synthesis inhibitor, such as abiraterone acetate, for instance ZYTIGA®.

In certain aspects, an antibody of the present disclosure is combined with an androgen synthesis inhibitor, such as abiraterone acetate. In certain aspects, said antibody is a bispecific antibody as disclosed herein. In certain aspects, abiraterone acetate used in the prior cancer treatment as well as further included in the present method of treatment is administered at a daily dose of 1000 mg.

In certain aspects, abiraterone acetate, such as ZYTIGA®, is administered at a daily dose of 1000 mg. In certain aspects, abiraterone acetate is used in combination with an oral administration of prednisone at 5 mg twice daily. In certain aspects, abiraterone acetate, such as ZYTIGA®, is administered at a daily dose of 1000 mg and the bispecific antibody of the present disclosure is administered in an amount of 750 mg once every two weeks. In certain aspects, said bispecific antibody is zenocutuzumab. Thus, treatment of said subject comprises administration of both abiraterone acetate and zenocutuzumab during the same treatment period.

Furthermore, the subject may have received further prior treatment against prostate cancer, including taxane-based chemotherapy, such as docetaxel or cabazitaxel. Additionally or alternatively, the subject may also have received prior treatment with sipuleucel-T or radiopharmaceuticals, such as radium-223 or lutetium LU 177.

Radiopharmaceuticals, or medicinal radio compounds, are a group of pharmaceutical drugs containing radioactive isotopes. Examples include Radium-223 and Lutetium (177Lu) oxodotreotide. Lutetium (¹⁷⁷Lu) oxodotreotide (International Nonproprietary Name or INN) or ¹⁷⁷Lu DOTA-TATE, trade name Lutathera®, is a chelated complex of a radioisotope of the element lutetium with DOTA-TATE, used in peptide receptor radionuclide therapy.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer). In certain aspects, the subject is a human subject. Herein, the subject has castration-resistant prostate cancer, or is at risk of developing castration-resistant prostate cancer.

As used herein, castration-resistant prostate cancer (CRPC) refers to subjects with prostate cancer which has progressed after prior prostate treatment and having testosterone levels at or below the level detected after a subject has undergone castration (also referred to as “testosterone at castration level”). In certain aspects, subjects show an increase in serum prostate-specific antigen and/or have developed new metastases and/or progression of existing metastases, while receiving androgen deprivation therapy (ADT). In certain aspects, subjects with CRPC have serum testosterone levels of <50 ng/dL.

Castration serves to suppress production of androgens, such as testosterone, which is one method used in treatment of prostate cancer to reduce circulating androgen levels. Castration is typically achieved using surgical (orchiectomy) or chemical means. Such chemical means include androgen deprivation therapy (ADT), chemotherapy (including, but not limited to, docetaxel (Taxotere®) or cabazitaxel (Jevtana®)), androgen receptor axis-targeted (ARAT) agents, androgen synthesis inhibitors and (second-generation) androgen receptor antagonists. Examples include, but are not limited to, abiraterone, enzalutamide, apalutamide and darolutamide. Further examples of AR antagonists are included in Table 1.

TABLE 1
Timeline for the development of AR
antagonists for prostate cancer.

| | | |
|---|---|---|
| Generic name | Other name | Treatments |

First-generation

| | | |
|---|---|---|
| Flutamide | Eulexin | mCRPC |
| Bicalutamide | Casodex | mCRPC |
| Nilutamide | Nilandron | mCRPC (combined with |
| | | surgical castration) |

Second-generation

| | | |
|---|---|---|
| Enzalutamide | MDV3100 | mCRPC |
| | | nmCRPC |
| | | mCSPC |
| Apalutamide | ARN-509 | nmCRPC |
| | | mCSPC/mCRPC |
| Darolutamide | ODM-201 | nmCRPC |

Candidates in clinical trials

| | | |
|---|---|---|
| Proxalutamide | GT-0918 | mCRPC |
| BMS-641988 | | CRPC |
| TQB3720 | | mCRPC |
| SHR3680 | Rezvilutamide | mCRPC |
| TRC-253 | | mCRPC |

In certain aspects, castration-resistant prostate cancer is a cancer that has progressed after having received a line of a second-generation hormonal agent for treatment of castration-resistant prostate cancer. In certain aspects, castration-resistant prostate cancer is a cancer that has progressed after having received not more than two lines of a second-generation hormonal agent for treatment of castration-resistant prostate cancer. Such second-generation hormonal agents include, but are not limited to, androgen receptor antagonists (including, but not limited to, enzalutamide), or androgen-synthesis inhibitors (including, but not limited to, abiraterone).

In certain aspects, the subject to be treated exhibits increasing levels of Prostate-Specific Antigen (PSA), defined as two increases in PSA reaching a minimum value of ≥1 ng/mL before start of treatment. In certain aspects, the increase is over a previous reference value. Increasing levels of Prostate-Specific Antigen (PSA) is typically an increase in PSA greater than 25% and >2 ng/ml above nadir, confirmed by progression at two timepoints at least three weeks apart.

In certain aspects, the patient may have had an interruption of dosing of an androgen receptor axis-targeting agent, such as abiraterone or enzalutamide, for more than 14 days, after which increasing PSA levels are documented after resuming with said agent.

Herein the term “androgen receptor axis-targeting agent” includes androgen synthesis inhibitors and androgen receptor antagonists. As such, an androgen receptor axis-targeting agent as meant herein prevents, blocks or reduces signaling activity of the androgen receptor. Activity of the androgen receptor axis is involved in progression into castration-resistant prostate cancer. Said effect on the androgen receptor axis can be achieved using an androgen synthesis inhibitor, such as abiraterone acetate, which affects serum androgen levels, such as testosterone, such that androgen receptor activity is prevented, blocked or reduced. Alternatively, said effect on the androgen receptor axis can be achieved using an androgen receptor antagonist, such as enzalutamide, to prevent, block or reduce binding of ligand to the androgen receptor and its activation thereof.

At the start of treatment, at least one, more than one or all of the following inclusion factors IF1-IF7 are applicable to subjects for treatment. In certain aspects, the subject comprises or complies with all of inclusion factors IF1-IF7.

IF1. Having an age of equal to or more than 18 years.

IF2. Having an Eastern Cooperative Oncology Group performance status (ECOG) of 0 or 1.

IF3. Having an estimated life expectancy of equal to or more than 12 weeks.

IF4. Having a minimum of three weeks since any major surgery, completion of radiation, completion of all prior systemic anticancer therapy, or at least 5-half-lives if the prior therapy is a single-agent small molecule therapeutic agent, and adequately recovered from the acute toxicitys of any prior therapy to Grade equal to or less than 1 National Cancer Institute (NCI)-Common Terminology Criteria for AEs (CTCAE) v. 5.0, except in cases of alopecia or neuropathy. Such a single-agent small molecule therapeutic agent may be flutamide, ketoconazole and the like.

IF5. Having a Left Ventricular Ejection Fraction (LVEF) equal to or more than 50% by echocardiogram (ECHO) or multigated acquisition scan (MUGA).

IF6. Having adequate organ function as established by:
IF6.1. An absolute neutrophil count of equal to or more than 1.5×10⁹/L.
IF6.2. A hemoglobin level of equal to or more than 9 g/dL.
IF6.3. A platelets count of equal to or more than 100×10⁹/L.
IF6.4. Serum calcium within normal ranges (or corrected with supplements).
IF6.5. Alanine aminotransferase (ALT) and aspartate aminotransferase (AST) equal to or less than 2.5× upper limit of normal (ULN), with the proviso that if a liver malignancy is involved, ALT/AST is equal to or less than 5×ULN.
IF6.6 A total bilirubin level of equal to or less than 1.5×ULN, with the proviso that total bilirubin is equal to or less than 3×ULN in case of Gilbert disease.
IF6.7 An estimated glomerular filtration rate of more than 30 mL/min based on the Cockroft-Gault formula, and
IF6.8 Serum albumin more than 3.0 g/dL.

IF7. Having a formalin-fixed paraffin embedded (FFPE) tumor specimen. The specimen may be collected de novo an archival FFPE tumor sample, preferably collected within 2 years of the start of treatment.

In certain aspects, all values for organ function measurements according to IF6 have an upper limit observed with healthy subjects.

In certain aspects, the subject for treatment comprises one or more factors selected from the group consisting of IF1-IF7. In certain aspects, the subject for treatment comprises factors IF2, IF3, IF5 and IF6. In certain aspects, the subject for treatment comprises all of the factors IF1-IF7.

Herein, ECOG Performance Status Scoring Grade Definition is as follows: 0 Fully active, able to carry on all pre-disease performance without restriction. 1 Restricted in physically strenuous activity but ambulatory and able to carry out work of a light or sedentary nature, eg, light housework, office work. 2 Ambulatory and capable of all self-care but unable to carry out any work activities. Up and about more than 50% of waking hours. 3 Capable of only limited self-care, confined to bed or chair more than 50% of waking hours. 4 Completely disabled. Cannot carry on any self-care. Totally confined to bed or chair. 5 Dead.

In certain aspects, the subject to be treated has histologically confirmed adenocarcinoma of the prostate, in certain aspects without neuroendocrine differentiation or small cell features.

In certain aspects, the subject to be treated has metastatic disease documented by at least 2 bone lesions on whole body bone scintigraphy, or soft tissue disease documented by computed tomography (CT) scan/magnetic resonance imaging (MRI).

In certain aspects, the subject to be treated has a serum testosterone level of ≤1.73 nmol/L (≤50 ng/dL) prior to the start of treatment. In certain aspects, the subject for treatment is undergoing androgen deprivation treatment. In certain aspects, the subject for treatment is undergoing androgen deprivation treatment with a serum testosterone level of ≤1.73 nmol/L (≤50 ng/dL) prior to treatment. Hence, in certain aspects, the method of treatment is preceded by a step of determining serum testosterone levels on a sample obtained from said subject. Subsequently, said subject may be selected for treatment if serum testosterone levels are ≤1.73 nmol/L (≤50 ng/dL). The use of testosterone detection assays with a sensitivity of 1 to 2 ng/dL is recommended by the PCWG3 guidelines when determining serum testosterone levels and such detection assays are readily available.

In certain aspects, prior treatment with an androgen receptor axis-targeting agent started at least 90 days or at least 118 days prior to start of the treatment according to the present disclosure. An interruption of dosing of a maximum of 30 days is permitted during the at least 90 or 118 days.

In certain aspects, the subject to be treated has CRPC or CRPC disease progression according to Prostate Cancer Working Group 3 (PCWG3) criteria. In certain aspects, CRPC disease progression is established in accordance with at least one or more of the following criteria:
1. Increasing levels of Prostate-Specific Antigen (PSA), which is defined as two increases in PSA over a previous reference value reaching a minimum value of ≥1 ng/mL before start of treatment, with the proviso that if the patient had an interruption of dosing of an androgen receptor axis-targeting agent, such as abiraterone or enzalutamide, for more than 14 days, increasing PSA levels are documented after resuming with said agent.
2. Soft tissue disease progression as defined by RECIST v1.1.
3. Progression of previously normal (<10 mm) lymph nodes, as determined by growth of >5 mm over the short axis.
4. Bone disease progression defined by two or more new lesions as shown by whole body bone scintigraphy.

Increasing, or determining said increasing levels of, Prostate-Specific Antigen (PSA) is an increase in PSA greater than 25% and >2 ng/ml above nadir, confirmed by progression at two timepoints at least three weeks apart. (See Scher et al., 2016, Trial Design and Objectives for Castration-Resistant Prostate Cancer: Updated Recommendations From the Prostate Cancer Clinical Trials Working Group 3.” J Clin Oncol 34(12): 1402-1418.)

In certain aspects, subjects to be treated are receiving a stable dose for a period of at least 4 weeks of bisphosphonates or denosumab. Said bisphosphonates or denosumab may be provided for maintaining or improving bone health.

In certain aspects, the subject to be treated is capable of swallowing oral medications and have absence of gastrointestinal conditions (eg, malabsorption, resection) deemed to jeopardize intestinal absorption.

In certain aspects, premedication with the following substances is provided:
Aracetamol/acetaminophen 1000 mg PO or IV.
Dexchlorpheniramine 5 mg IV (or other anti-H1 equivalent, PO or IV)
Dexamethasone 10 mg IV (or equivalent, PO or IV). If needed, corticosteroids are administered before Cycle 1 Day 1 administration and should be used for subsequent injections at the Investigator's discretion to manage infusion-related reactions (IRRs).
An H2 antagonist can be given at the Investigator's discretion.

In certain aspects, the subject to be treated has not received more than two lines of a second-generation hormonal agent for metastatic disease. Such agents include abiraterone and enzalutamide. In certain aspects, the subject to be treated has not received more than two lines of systemic chemotherapy for metastatic disease. Such chemotherapy includes docetaxel (Taxotere®) and cabazitaxel (Jevtana®). In certain aspects, the subject to be treated has not received a prior anti-HER3-directed therapy.

In certain aspects, the subject shows clinical efficacy according to PCWG3-modified RECIST v1.1 criteria after having received anti-CRPC treatment according to the present disclosure. In certain aspects, the patient shows progression-free survival (PFS), non-progressive disease (Non-PD), no evidence of disease (NED), stable disease (SD), partial response (PR) or complete response (CR) per PCWG3-modified RECIST v1.1 criteria. In certain aspects, the patient shows radiographic disease control measurable for lymph nodes, visceral tissue and/or bone tissue per PCWG3 or PCWG3-modified RECIST v1.1 criteria. In certain aspects, the subject shows said clinical efficacy after having received treatment with a bispecific antibody that comprises an antigen binding site that can bind an extracellular part of ERBB2 and an antigen binding site that can bind an extracellular part of ERBB3 or with an antibody that comprises an antigen binding site that can bind an extracellular part of ERBB3 to the subject. In certain aspects, said method of treatment further comprises the administration of an androgen receptor axis-targeting agent to the subject, like abiraterone or enzalutamide.

The terms “treat,” “treating,” and “treatment,” as used herein, refer to any type of intervention or process performed on or administering an active agent or combination of active agents to a subject with the objective of curing or improving a disease or symptom thereof. This includes reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease.

Any aspect disclosed herein as mentioned in relation to any use in a method of treatment according to the present disclosure applies equally to any method of treatment according to the present disclosure, and vice versa.

Any aspect disclosed herein as mentioned in relation to any use in a method of treatment according to the present disclosure applies equally to any use for the manufacture of a medicament for treatment according to the present disclosure, and vice versa.

Any aspect disclosed herein as mentioned in relation to any method of treatment according to the present disclosure applies equally to any use for the manufacture of a medicament for treatment according to the present disclosure, and vice versa.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The terms “therapeutic amount” or “effective amount” are used interchangeably herein and refer to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In certain aspects, a therapeutic amount is an amount sufficient to delay tumor development. In certain aspects, a therapeutic amount is an amount sufficient to prevent or delay tumor recurrence.

The therapeutic amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

A therapeutic amount may vary according to factors such as the disease state, age, sex, and weight of the individual to be treated, and the ability of the agent or combination of agents to elicit a desired response in the individual.

A therapeutic amount can be administered in one or more administrations.

A therapeutic amount also includes an amount that balances any toxic or detrimental effects of the agent or combination of agents and the therapeutically beneficial effects.”

Of note, loss of tumor suppressor genes is particularly common in CRPC and is associated with aggressive disease and poor prognosis. For effective inhibition of tumor growth mediated by a bispecific antibody as disclosed herein, such as zenocutuzumab, or by an ERBB3-specific antibody as disclosed herein, an intact signaling pathway downstream of the PI3K pathway may be necessary. Hence, in certain aspects, the castration-resistant prostate cancer to be treated does not have PTEN loss, or does not have PTEN deficiency. In certain aspects, a cancer to be treated according to the present disclosure is characterized as having wildtype PTEN status. In certain aspects, the cancer does not have PTEN loss. In certain aspects, the cancer is wildtype for PTEN. In certain aspects, the cancer does not exhibit PTEN loss. Without being bound by theory, in certain aspects classifying subjects on the basis of PTEN status is useful prior to commencing treatment as disclosed herein as cancers with appropriate PTEN status may benefit more from said treatment. Subjects for which PTEN status has been determined to be either wildtype PTEN or not having PTEN loss may be selected for treatment as disclosed herein or classified as being eligible for said treatment.

Alternatively, in certain aspects, a castration-resistant prostate cancer to be treated according to the present disclosure exhibits PTEN loss, or PTEN deficiency. In certain aspects, a cancer to be treated according to the present disclosure is characterized as having PTEN loss status. In certain aspects, the cancer has PTEN loss. In certain aspects, the cancer is not wildtype for PTEN. In certain aspects, the cancer exhibits PTEN loss. Without being bound by theory, in certain aspects classifying subjects on the basis of PTEN status is useful prior to commencing treatment as disclosed herein as cancers with appropriate PTEN status may benefit more from said treatment. Subjects for which PTEN status has been determined to be either not wildtype for PTEN or having PTEN loss may be selected for treatment as disclosed herein or classified as being eligible for said treatment.

PTEN status is capable of being determined by means known to persons of ordinary skill in the art, and any suitable means can be used in conjunction with the methods of treatment or use with an antibody of the present disclosure, including in a combination therapy as described herein. In certain aspects, PTEN status is determined using IHC. In certain aspects, PTEN status is determined using a liquid biopsy assay.

Determining PTEN status can for instance be done using commercially available PTEN genetic tests on cells of a biopsy or a blood sample. Various methods are available and many are known in the art. One way is by means of PCR-amplification with primers spanning the PTEN cDNA or genomic DNA, followed by sequencing of the amplified nucleotide molecules. This can be implemented for mutations that are known to occur and affect PTEN activity. New mutations can also be detected readily through techniques known to those of ordinary skill in the art, including by next-generation DNA or RNA sequencing. Other ways to determine PTEN status is by ELISA.

Nucleic acid-based techniques are available to determine PTEN status include allele specific RNA based methodology including RT-PCR, Real-time PCR, Transcriptome analysis, Anchored multiplex PCR, nCounter, FISH, DNA-based methodologies including Hybrid capture-based next generation sequencing (NGS), Amplicon-based NGS, among other techniques available commercially. Also, PTEN loss can be established using protein-based assays, such as immunostaining, IHC, FISH or the like. To date, robust clinical assays exit that use immunohistochemistry and fluorescence in situ hybridization to reproducibly measure PTEN protein and gene loss using diagnostic tissue biopsies and circulating tumour cells from plasma. Especially, IHC protocols have been successfully validated on the Ventana Benchmark platform in a Clinical Laboratory Improvement Amendments-certified laboratory with high interobserver reproducibility in the scoring system.

One example of a commercial kit to establish PTEN status is by use of the Guardant360® TissueNext® which is an analytically validated tissue comprehensive genomic profiling panel that includes TMB, MSI status, and PD-L1 IHC. A Guardant360 TissueNext report includes 84 genes. This kit allows genotyping point mutations (SNVs) and deletion variants (Indels) of 84 genes, including PTEN, amplification of 20 genes and identification of fusions of 12 genes. A further example of a commercial kit that can be used to establish PTEN status is the Roche Ventana Optiview kit DAB using SP218 PTEN antibody.

Hence, the present disclosure also provides a method of selecting a subject having castration-resistant prostate cancer for treatment with a bispecific antibody that comprises an antigen binding site that can bind an extracellular part of ERBB2 and an antigen binding site that can bind an extracellular part of ERBB3, the method comprising: a) determining PTEN status in a sample obtained from the subject; and b) selecting the subject for said treatment if the sample does not exhibit PTEN loss.

Alternatively, the present disclosure provides a method of establishing whether a subject having castration-resistant prostate cancer is likely to respond to treatment with a bispecific antibody that comprises an antigen binding site that can bind an extracellular part of ERBB2 and an antigen binding site that can bind an extracellular part of ERBB3, the method comprising: a) determining PTEN status in a sample obtained from the subject; and b) selecting a sample for not exhibiting PTEN loss, thereby establishing that the subject from which, or from whom, the sample is derived is likely to respond to said treatment.

Alternatively, the present disclosure provides a method of classifying a subject having castration-resistant prostate cancer on the basis of PTEN status prior to treatment with a bispecific antibody that comprises an antigen binding site that can bind an extracellular part of ERBB2 and an antigen binding site that can bind an extracellular part of ERBB3, the method comprising: a) determining PTEN status in a sample obtained from the subject; and b) classifying the subject from which, or from whom, the sample was obtained as eligible for said treatment if the sample does not exhibit PTEN loss.

The present disclosure also provides a method of selecting a subject having castration-resistant prostate cancer for treatment with a bispecific antibody that comprises an antigen binding site that can bind an extracellular part of ERBB2 and an antigen binding site that can bind an extracellular part of ERBB3, the method comprising: a) determining PTEN status in a sample obtained from the subject; and b) selecting the subject for said treatment if the sample exhibits PTEN loss.

Alternatively, the present disclosure provides a method of establishing whether a subject having castration-resistant prostate cancer is likely to respond to treatment with a bispecific antibody that comprises an antigen binding site that can bind an extracellular part of ERBB2 and an antigen binding site that can bind an extracellular part of ERBB3, the method comprising: a) determining PTEN status in a sample obtained from the subject; and b) selecting a sample for exhibiting PTEN loss, thereby establishing that the subject from which, or from whom, the sample is derived is likely to respond to said treatment.

Alternatively, the present disclosure provides a method of classifying a subject having castration-resistant prostate cancer on the basis of PTEN status prior to treatment with a bispecific antibody that comprises an antigen binding site that can bind an extracellular part of ERBB2 and an antigen binding site that can bind an extracellular part of ERBB3, the method comprising: a) determining PTEN status in a sample obtained from the subject; and b) classifying the subject from which, or from whom, the sample was obtained as eligible for said treatment if the sample exhibits PTEN loss.

In certain aspects, PTEN loss or PTEN status is determined using IHC. In another aspect, PTEN status is determined using a liquid biopsy assay which includes DNA sequencing.

In certain aspects, the cancer or subject to be treated according to the disclosure does not have an oncogenic driver mutation. In certain aspects, said cancer or subject does not have an oncogenic driver mutation in the PI3K, AKT, mTOR pathways. In certain aspects, said cancer or subject does not have an upregulation of said pathways. In certain aspects, said cancer or subject does not have a mutation in any one of EGFR, cMET, ALK, BRAF, KRAS, NRAS, RET and ROS1. In certain aspects, said cancer or subject does not have a mutation in known tumor-associated genes or the proteins encoded therefrom, such as EGFR, cMET, ALK, BRAF, KRAS, NRAS, RET and ROS1.

In certain aspects, said subject's cancer is tested by next generation sequencing (for instance DNA, RNA or whole transcriptome), and said cancer or subject is subsequently selected for treatment with a bispecific antibody that comprises an antigen binding site that can bind an extracellular part of ERBB2 and an antigen binding site that can bind an extracellular part of ERBB3, using a method comprising the steps of: a) determining the existence of an oncogenic driver mutation, in certain aspects a mutation that modulates the PI3K, AKT, and/or mTOR pathways, in a sample obtained from the subject; and b) classifying the subject from which, or from whom, the sample was obtained as eligible for said treatment if the sample lacks the existence of an oncogenic driver mutation, in certain aspects a mutation that modulates the PI3K, AKT, and/or mTOR pathways.

In certain aspects, said subject's cancer is tested by next generation sequencing (for instance DNA, RNA or whole transcriptome sequencing), and said cancer or subject is subsequently selected for treatment with a bispecific antibody that comprises an antigen binding site that can bind an extracellular part of ERBB2 and an antigen binding site that can bind an extracellular part of ERBB3, using a method comprising the steps of: a) determining the existence of an oncogenic driver mutation, in certain aspects an oncogenic driver mutation in any one of EGFR, cMET, ALK, BRAF, KRAS, NRAS, RET and ROS1 in a sample from said cancer of said subject; and b) classifying the subject from which, or from whom, said sample was obtained as eligible for said treatment if said sample lacks the existence of an oncogenic driver mutation, in certain aspects in EGFR, cMET, ALK, BRAF, KRAS, NRAS, RET and ROS1.

In certain aspects, a CRPC or a subject having a CRPC is selected for treatment with a bispecific antibody that comprises an antigen binding site that can bind an extracellular part of ERBB2 and an antigen binding site that can bind an extracellular part of ERBB3, using a method comprising the steps of: a) determining the existence of an oncogenic driver mutation, in certain aspects a mutation that modulates the PI3K, AKT, and/or mTOR pathways, in a sample obtained from the CRPC of the subject; and b) classifying the subject from which, or from whom, the sample was obtained as eligible for said treatment if the sample lacks the existence of an oncogenic driver mutation, in certain aspects a mutation that modulates the PI3K, AKT, and/or mTOR pathways.

In certain aspects, a CRPC or a subject having a CRPC is selected for treatment with a bispecific antibody that comprises an antigen binding site that can bind an extracellular part of ERBB2 and an antigen binding site that can bind an extracellular part of ERBB3, using a method comprising the steps of: a) determining the existence of an oncogenic driver mutation, in certain aspects an oncogenic driver mutation in any one of EGFR, cMET, ALK, BRAF, KRAS, NRAS, RET and ROS1 in a sample obtained from the CRPC of the subject; and b) classifying the subject from which, or from whom, the sample was obtained as eligible for said treatment if the sample lacks the existence of an oncogenic driver mutation, in certain aspects in EGFR, cMET, ALK, BRAF, KRAS, NRAS, RET and ROS1.

In certain aspects, the sample obtained from said subject is a sample taken from the tumor or cancer prior to the anti-CRPC treatment of the present disclosure. In certain aspects, the sample obtained from said subject is a sample that comprises a tumor or cancer cell from the tumor or cancer from which the subject to be treated is suffering. In certain aspects, the sample taken from the tumor or cancer is obtained prior to commencing anti-CRPC treatment of the present disclosure.

As used herein, the term “antigen-binding site” refers to a site derived from and preferably as present on a bispecific antibody which is capable of binding to antigen. An antigen-binding site is typically formed by and present in the variable domain of the antibody. The variable domain contains said antigen-binding site. The antigen-binding site can bind to the antigen under normal physiological conditions. This is often also referred to as that the antigen binding site “binds” the antigen.

In one embodiment an antibody variable domain comprises a heavy chain variable region (VH) and a light chain variable region (VL). The antigen-binding site can be present in the combined VH/VL variable domain, or in only the VH region or only the VL region. When the antigen-binding site is present in only one of the two regions of the variable domain, the counterpart variable region can contribute to the folding and/or stability of the binding variable region, but does not significantly contribute to the binding of the antigen itself.

As used herein, antigen-binding refers to the typical binding capacity of an antibody to its antigen. An antibody comprising an antigen-binding site that binds to ERBB2, binds to ERBB2 and, under otherwise identical conditions, at least 100-fold lower to the homologous receptors ERBB1 and ERBB4 of the same species. An antibody comprising an antigen-binding site that binds to ERBB3, binds to ERBB3 and, under otherwise identical conditions, not to the homologous receptors ERBB1 and ERBB4 of the same species.

Considering that the ERBB-family is a family of cell surface receptors, the binding is typically assessed on cells that express the receptor(s). Binding of an antibody to an antigen can be assessed in various ways. One way is to incubate the antibody with the antigen (preferably cells expressing the antigen), removing unbound antibody (preferably by a wash step) and detecting bound antibody by means of a labeled antibody that binds to the bound antibody.

Antigen binding by an antibody is typically mediated through the complementarity regions of the antibody and the specific three-dimensional structure of both the antigen and the variable domain allowing these two structures to bind together with precision (an interaction similar to a lock and key), as opposed to random, non-specific sticking of antibodies. As an antibody typically recognizes an epitope of an antigen, and as such epitope may be present in other compounds as well, antibodies according to the present invention that bind ERBB2 and/or ERBB3 may recognize other proteins as well, if such other compounds contain the same epitope. Hence, the term “binding” does not exclude binding of the antibodies to another protein or protein(s) that contain the same epitope.

Such other protein(s) is preferably not a human protein. An ERBB2 antigen-binding site and an ERBB3 antigen-binding site as defined herein typically do not bind to other proteins on the membrane of cells in a post-natal, preferably adult human.

The term “interferes with binding” as used herein means that the antibody is directed to an epitope on ERBB3 and the antibody competes with ligand for binding to ERBB3. The antibody may diminish ligand binding, displace ligand when this is already bound to ERBB3 or it may, for instance through steric hindrance, at least partially prevent that ligand can bind to ERBB3.

The term “antibody” as used herein means a proteinaceous molecule, preferably belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity. The specificity determines which antigen or epitope thereof is specifically bound by the binding domain. The affinity is a measure for the strength of binding to a particular antigen or epitope. Antibodies such the bispecific antibodies of the present invention comprise the constant domains (Fc part) of a natural antibody. An antibody of the invention is typically a bispecific full length antibody, preferably of the human IgG subclass. Preferably, an antibody as disclosed herein is of the human IgG1 subclass. Such antibodies have good ADCC properties, have favorable half-life upon in vivo administration to humans and CH3 engineering technology exists that can provide for modified heavy chains that preferentially form heterodimers over homodimers upon co-expression in clonal cells.

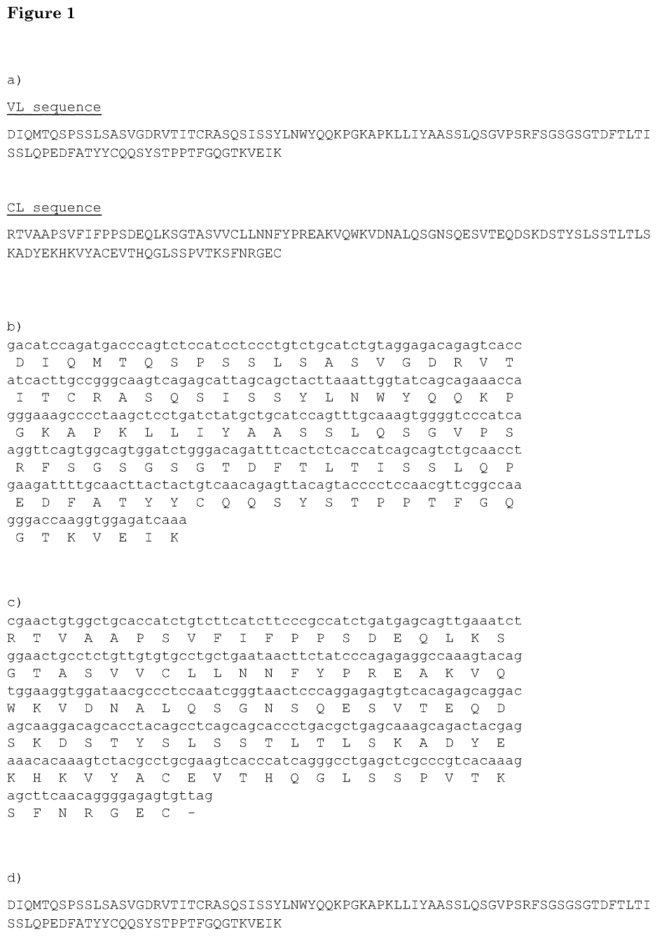

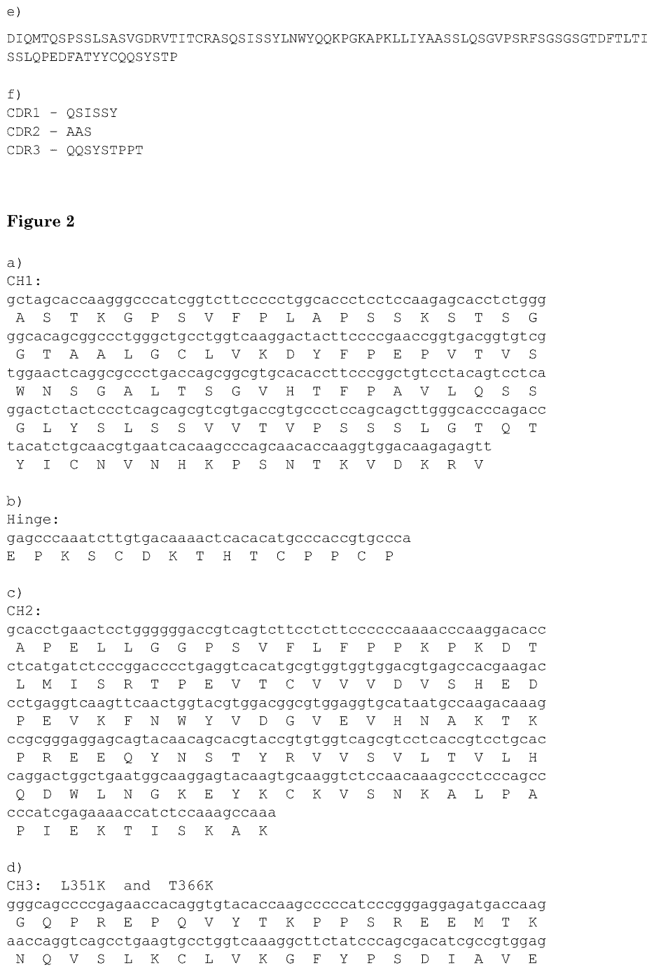

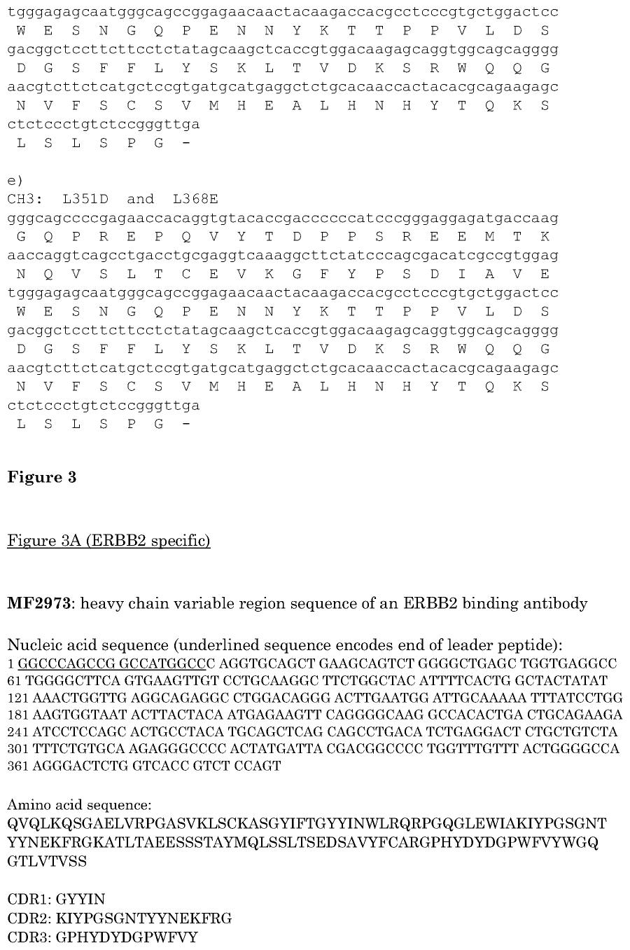

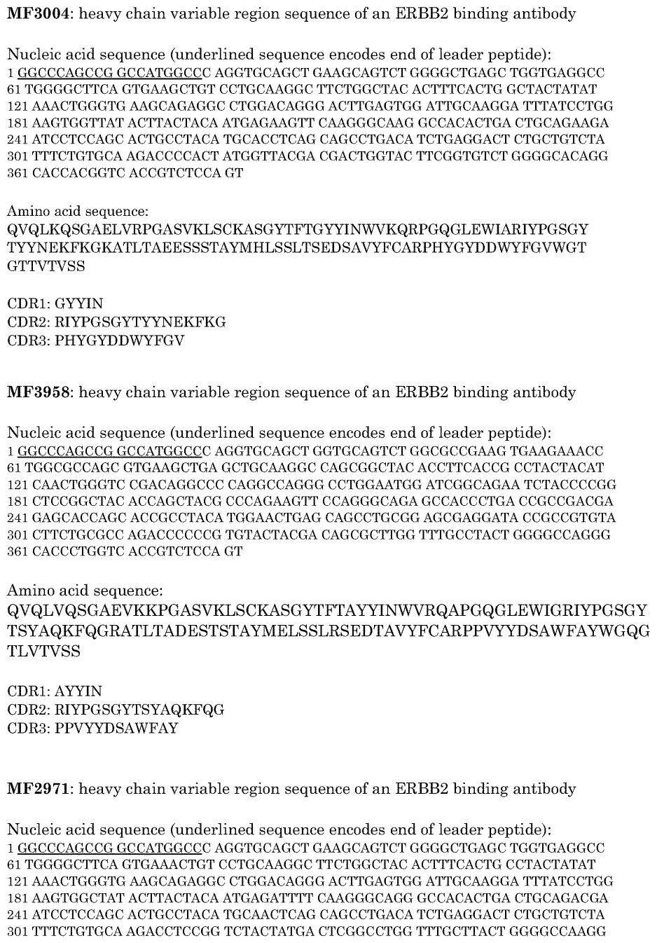

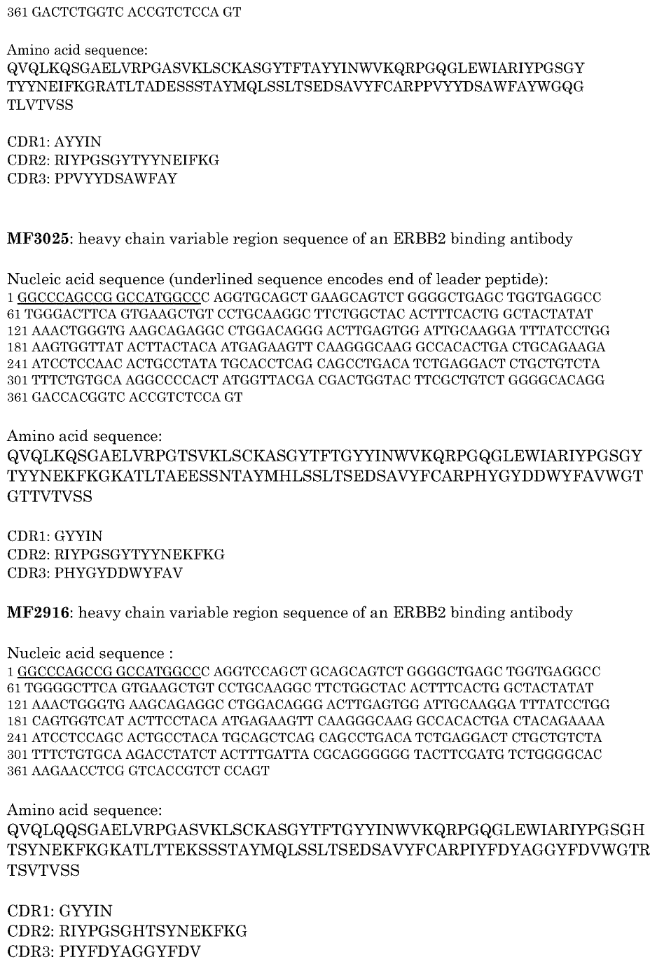

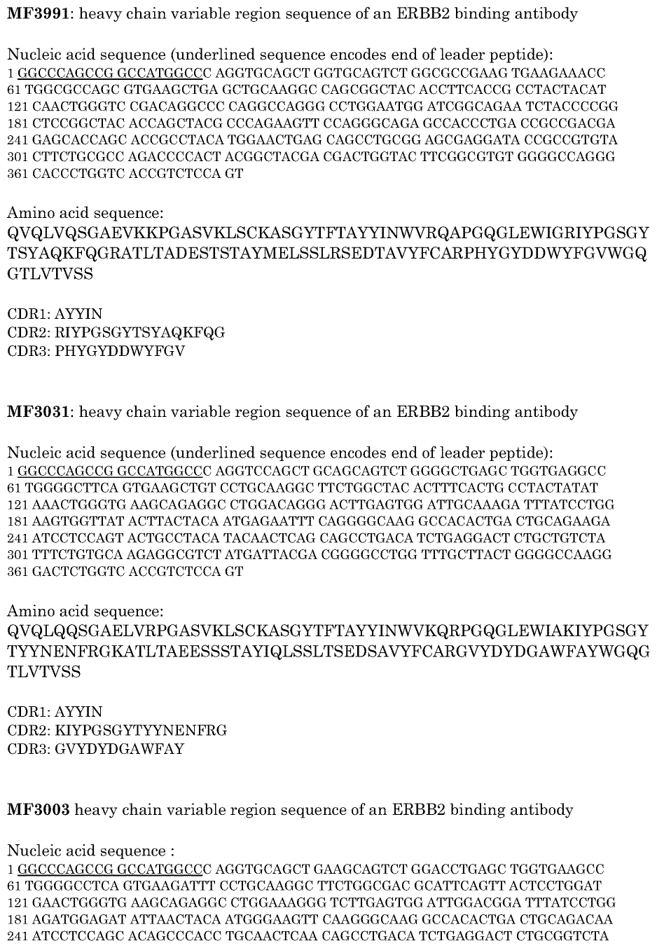

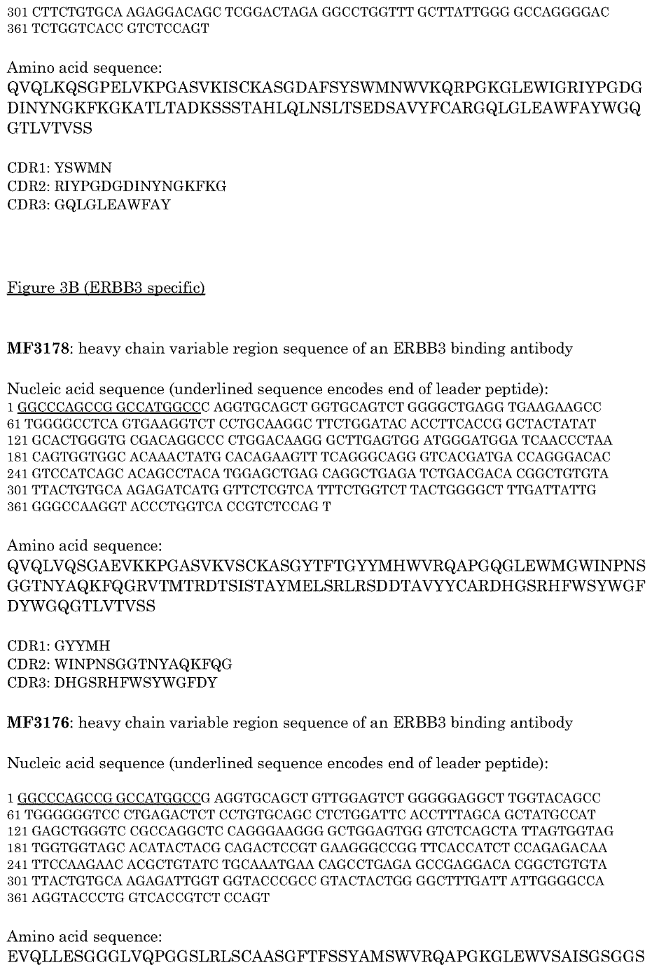

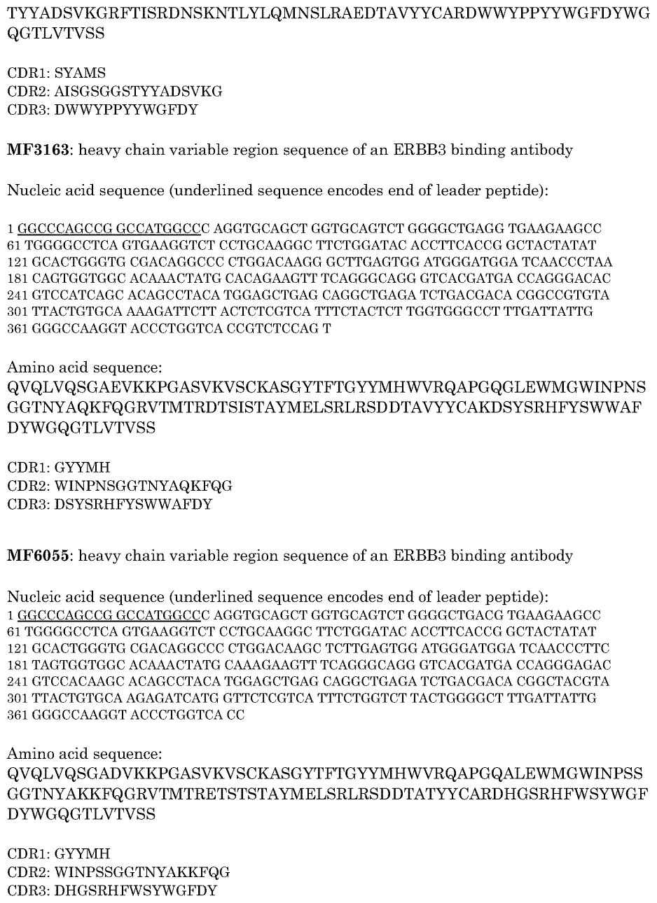

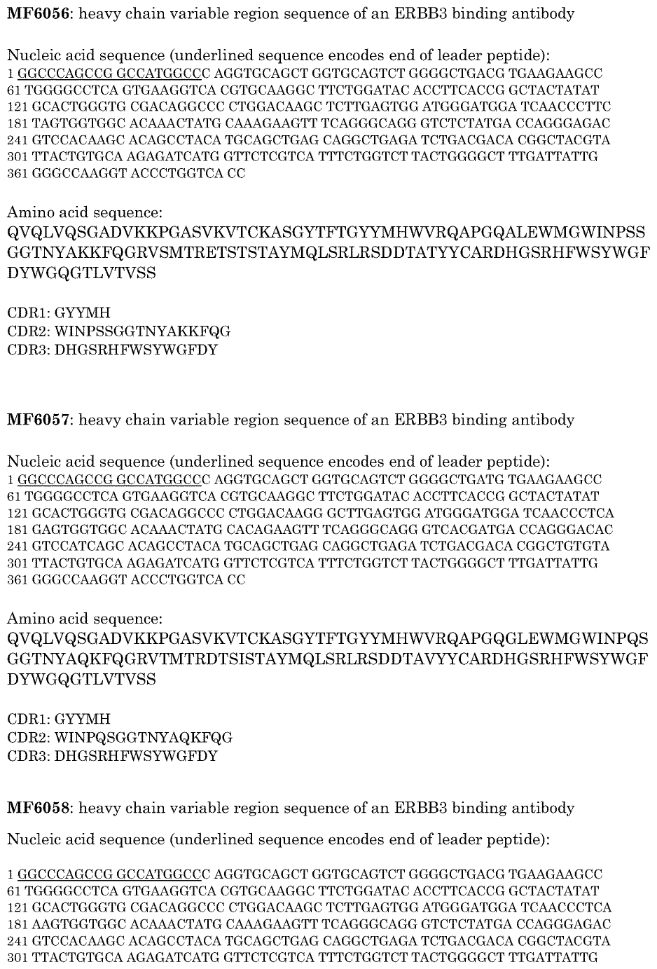

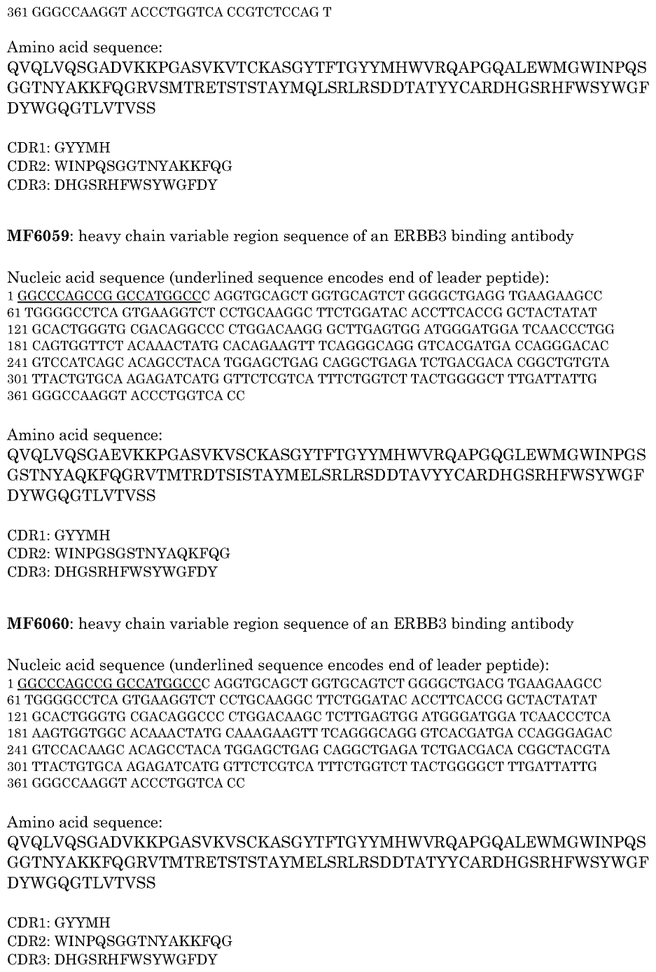

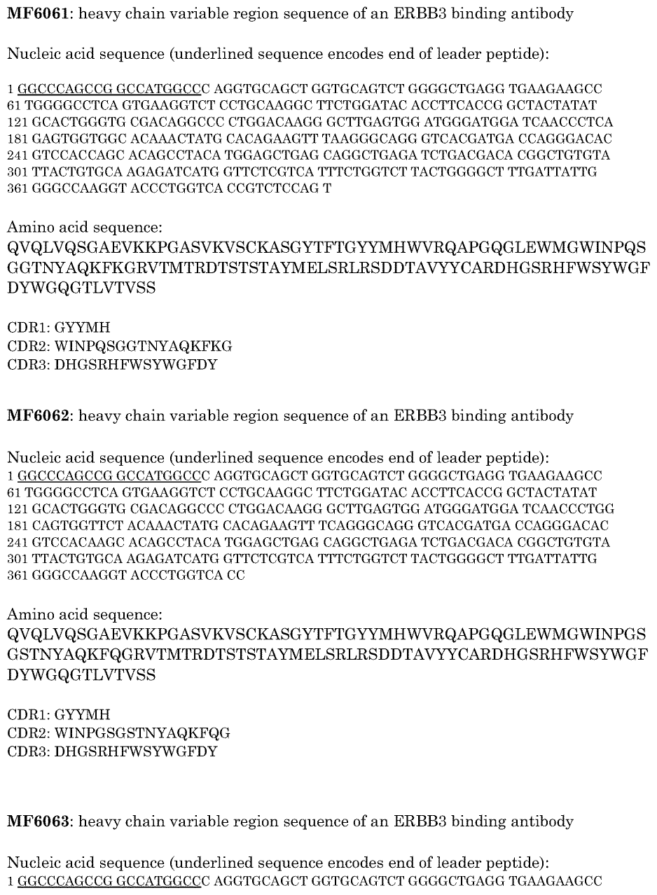

An antibody as disclosed herein is preferably a “full length” antibody. The term ‘full length’ is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL (suitable amino acid sequences for the respective domains are depicted in FIG. 1 and FIG. 2. An antibody binds to antigen via the variable domains contained in the Fab portion, and after binding can interact with molecules and cells of the immune system through the constant domains, mostly through the Fc portion. The terms ‘variable domain’, ‘VH/VL pair’, ‘VH/VL’ are used herein interchangeably. Full length antibodies according to the invention encompass antibodies wherein mutations may be present that provide desired characteristics. Such mutations should not be deletions of substantial portions of any of the regions. However, antibodies wherein one or several amino acid residues are deleted, without essentially altering the binding characteristics of the resulting antibody are embraced within the term “full length antibody”. For instance, an IgG antibody can have 1-20 amino acid residue insertions, deletions or a combination thereof in the constant region. For instance, ADCC activity of an antibody can be improved when the antibody itself has a low ADCC activity, by slightly modifying the constant region of the antibody (Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489).

Full length IgG antibodies are preferred because of their favorable half-life and the need to stay as close to fully autologous (human) molecules for reasons of immunogenicity. An antibody as disclosed herein is preferably a bispecific IgG antibody, preferably a bispecific full length IgG1 antibody. IgG1 is favored based on its long circulatory half-life in man. In order to prevent any immunogenicity in humans it is preferred that the bispecific IgG antibody is a human IgG1.

The term ‘bispecific’ (bs) means that one part of the antibody (as defined above) binds to one epitope on an antigen whereas a second part binds to a different epitope. The different epitope is typically present on a different antigen. The heavy chain variable regions of the bispecific antibody are typically different from each other, whereas the light chain variable regions are preferably the same. A bispecific antibody wherein the different heavy chain variable regions are associated with the same, or a common, light chain is also referred to as a bispecific antibody with a common light chain. A bispecific antibody as described herein typically comprises one variable domain that binds ERBB2 and another variable domain that binds ERBB3.

Preferred bispecific antibodies can be obtained by co-expression of two different heavy chains and a common light chain in a single cell. When wildtype CH3 domains are used, co-expression of two different heavy chains and a common light chain will result in three different species, AA, AB and BB. To increase the percentage of the desired bispecific product (AB) CH3 engineering can be employed, or in other words, one can use heavy chains with compatible heterodimerization domains, as defined hereunder. Suitable compatible CH3 heterodimerization domains are depicted in FIGS. 2d and 2e.

The term ‘compatible heterodimerization domains’ as used herein refers to protein domains that are engineered such that engineered domain A′ will preferentially form heterodimers with engineered domain B′ and vice versa, whereas homodimerization between A′-A′ and B′-B′ is diminished.

The term ‘common light chain’ refers to light chains which may be identical or have some amino acid sequence differences while the binding specificity of the full length antibody is not affected. It is for instance possible, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common VL’, ‘single light chain’, ‘single VL’, with or without the addition of the term ‘rearranged’ are all used herein interchangeably.

A common light chain (variable region) preferably has a germline sequence. A preferred germline sequence is a light chain variable region that is frequently used in the human repertoire and has good thermodynamic stability, yield and solubility. In a preferred embodiment the light chain comprises a light chain region comprising the amino acid sequence of an IgVκ1-39*01 gene segment as depicted FIG. 1, more-preferably common light chain IGKV1-39/jk1 with 0-10, preferably 0-5 amino acid insertions, deletions, substitutions, additions or a combination thereof. IgVκ1-39 is short for Immunoglobulin Variable Kappa 1-39 Gene. The gene is also known as Immunoglobulin Kappa Variable 1-39; IGKV139; or IGKV1-39. External Ids for the gene are HGNC: 5740; Entrez Gene: 28930; Ensembl: ENSG00000242371. The variable region of IGKV1-39 is listed in the FIG. 1. The V-region can be combined with one of five J-regions. FIG. 1 describes two preferred sequences for IgVκ1-39 in combination with a J-region. The joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org).

It is preferred that the IgVκ1-39*01 comprising light chain variable region is a germline sequence. It is further preferred that the IGJκ1*01 or/IGJκ5*01 comprising light chain variable region is a germline sequence. In a preferred embodiment, the IGKV1-39/jk1 or IGKV1-39/jk5 light chain variable regions are germline sequences.

In a preferred embodiment the light chain variable region comprises a germline IgVκ1-39*01. In a preferred embodiment the light chain variable region comprises the kappa light chain IgVκ1-39*01/IGJκ1*01 or IgVκ1-39*01/IGJκ5*01. In a preferred embodiment a IgVκ1-39*01/IGJκ1*01. The light chain variable region preferably comprises a germline kappa light chain IgVκ1-39*01/IGJκ1*01 or germline kappa light chain IgVκ1-39*01/IGJκ5*01, preferably a germline IgVκ1-39*01/IGJκ1*01.

Those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions. The light chain can also be a light chain as specified herein above, having 1-5 amino acid insertions, deletions, substitutions or a combination thereof.

Preferably, both the first antigen binding site and said second antigen binding site comprise a light chain variable region comprising a CDR1 having the sequence (RASQSISSYLN), a CDR2 having the sequence (AASSLQS), and a CDR3 having the sequence (QQSYSTPPT) according to KABAT numbering or according to the IMGT numbering system, the CDRs are QSISSY, AAS and QQSYSTPPT, respectively.

Antibodies disclosed herein can reduce a ligand-induced receptor function of ERBB3 on an ERBB2 and ERBB3 positive cell. In the presence of excess ERBB2, ERBB2/ERBB3 heterodimers may provide a growth signal to the expressing cell in the absence of detectable ligand for the ERBB3 chain in the heterodimer. This ERBB3 receptor function is herein referred as a ligand-independent receptor function of ERBB3. The ERBB2/ERBB3 heterodimer also provide a growth signal to the expressing cell in the presence of an ERBB3 ligand. This ERBB3 receptor function is herein referred to as a ligand-induced receptor function of ERBB3.

The term “ERBB3 ligand” as used herein refers to polypeptides which bind and activate ERBB3. Examples of ERBB3 ligands include, but are not limited to neuregulin 1 (NRG) and neuregulin 2, betacellulin, heparin-binding epidermal growth factor, and epiregulin. The term includes biologically active fragments and/or variants of a naturally occurring polypeptide.

Preferably, the ligand-induced receptor function of ERBB3 is ERBB3 ligand-induced growth of an ERBB2 and ERBB3 positive cell. In a preferred embodiment said cell is an MCF-7 cell (ATCC® HTB-22™); an SKBR3 (ATCC® HTB-30™) cell; an NCI-87 (ATCC® CRL-5822™) cell; a BxPC-3-luc2 cell (Perkin Elmer 125058), a BT-474 cell (ATCC® HTB-20™) or a JIMT 1 cell (DSMZ no.: ACC 589).

The ERBB2 protein contains several domains (see for reference FIG. 1 of Landgraf, R Breast Cancer Res. 2007; 9(1): 202-). The extracellular domains are referred to as domains I-IV. The place of binding to the respective domains of antigen-binding sites of antibodies described herein has been mapped. A bispecific antibody with an antigen-binding site (first antigen-binding site) that binds domain I or domain IV of ERBB2 (first antigen-binding site) comprises a heavy chain variable region that maintains significant binding specificity and affinity for ERBB2 when combined with various light chains. Bispecific antibodies with an antigen-binding site (first antigen-binding site) that binds domain I or domain IV of ERBB2 (first antigen-binding site) and an antigen-binding site for ERBB3 (second antigen-binding site) are more effective in reducing a ligand-induced receptor function of ERBB3 when compared to a bispecific antibody comprising an antigen-binding site (first antigen-binding site) that binds to another extra-cellular domain of ERBB2. A bispecific antibody comprising an antigen-binding site (first antigen-binding site) that binds ERBB2, wherein said antigen-binding site binds to domain I or domain IV of ERBB2 is preferred. Preferably said antigen-binding site binds to domain IV of ERBB2. Preferred antibodies comprise a first antigen-binding site that binds domain I of ERBB2 and a second antigen-binding site that binds domain III of ERBB3.

In one preferred embodiment, said antibody comprises an antigen-binding site that binds at least one amino acid of domain I of ERBB2 selected from the group consisting of T144, T164, R166, P172, G179, S180 and R181, and surface-exposed amino acid residues that are located within about 5 amino acid positions from T144, T164, R166, P172, G179, S180 or R181.

In one preferred embodiment, said antibody preferably comprises an antigen-binding site that binds at least one amino acid of domain III of ERBB3 selected from the group comprising R426 and surface-exposed amino acid residues that are located within 11.2 Å from R426 in the native ERBB3 protein.

A bispecific antibody with an antigen-binding site (first antigen-binding site) that binds ERBB2, and that further comprises ADCC are more effective than other ERBB2 binding antibodies that did not have significant ADCC activity, particularly in vivo. A bispecific antibody which exhibits ADCC is therefore preferred. By engineering Fc regions (through introducing amino acid substitutions) that bind to activating receptors with greater selectivity, antibodies can be created that have greater capability to mediate cytotoxic activities desired by an anti-cancer Mab.

One technique for enhancing ADCC of an antibody is afucosylation. (See for instance Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489). Further provided is therefore a bispecific antibody as disclosed herein, which is afucosylated. Alternatively, or additionally, multiple other strategies can be used to achieve ADCC enhancement, for instance including glycoengineering and mutagenesis, all of which seek to improve Fc binding to low-affinity activating FcγRIIIa, and/or to reduce binding to the low affinity inhibitory FcγRIIb.

Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are typically co-incubated with the antibody-labeled target cells. Target cell lysis is subsequently typically measured by release of intracellular label, for instance by a scintillation counter or spectrophotometry.

The antibodies disclosed herein are in certain aspects used in humans. Thus, in certain aspects said antibodies are human or humanized antibodies. Tolerance of a human to a polypeptide is governed by many different aspects. Immunity, be it T-cell mediated, B-cell mediated or other is one of the variables that are encompassed in tolerance of the human for a polypeptide. The constant region of a bispecific antibody is preferably a human constant region. The constant region may contain one or more, preferably not more than 10, preferably not more than 5 amino-acid differences with the constant region of a naturally occurring human antibody. It is preferred that the constant part is entirely derived from a naturally occurring human antibody. Various antibodies produced herein are derived from a human antibody variable domain library. As such these variable domains are human. The unique CDR regions may be derived from humans, be synthetic or derived from another organism. The variable region is considered a human variable region when it has an amino acid sequence that is identical to an amino acid sequence of the variable region of a naturally occurring human antibody, but for the CDR region. The variable region of an ERBB2 binding VH, an ERBB3 binding VH, or a light chain in an antibody may contain one or more, preferably not more than 10, preferably not more than 5 amino-acid differences with the variable region of a naturally occurring human antibody, not counting possible differences in the amino acid sequence of the CDR regions. Such mutations occur also in nature in the context of somatic hypermutation.

Antibodies may be derived from various animal species, at least with regard to the heavy chain variable region. It is common practice to humanize such e.g. murine heavy chain variable regions. There are various ways in which this can be achieved among which there are CDR-grafting into a human heavy chain variable region with a 3D-structure that matches the 3-D structure of the murine heavy chain variable region; deimmunization of the murine heavy chain variable region, preferably done by removing known or suspected T- or B-cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

Such deimmunized murine heavy chain variable regions are less immunogenic in humans than the original murine heavy chain variable region. Preferably a variable region or domain is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in the invention is preferably a mammal, more preferably a primate, most preferably a human.

In certain aspects, an antibody disclosed herein comprises a constant region of a human antibody. According to differences in their heavy chain constant domains, antibodies are grouped into five classes, or isotypes: IgG, IgA, IgM, IgD, and IgE. These classes or isotypes comprise at least one of said heavy chains that is named with a corresponding Greek letter. In certain aspects, the constant region comprises an IgG constant region, in certain aspects an IgG1 constant region, in certain aspects a mutated IgG1 constant region. Some variation in the constant region of IgG1 occurs in nature, such as for instance the allotypes G1m1, 17 and G1m3, and/or is allowed without changing the immunological properties of the resulting antibody. Typically between about 1-10 amino acid insertions, deletions, substitutions or a combination thereof are allowed in the constant region.

In certain aspects, an antibody of the present disclosure, including but not limited to a bispecific antibody, comprises an antigen binding site that can bind an extracellular part of ERBB3 which blocks both ERBB3 and its ligand heregulin. In certain aspects, said antibody also comprises an antigen binding site that can bind an extracellular part of ERBB3 at domain III. In certain aspects, said antibody also comprises an antigen binding site that can bind an extracellular part of ERBB2. In certain aspects, said antibody comprises an antigen binding site that can bind an extracellular part of ERBB2 and an antigen binding site that can bind an extracellular part of ERBB3. The bispecific antibody of the present disclosure comprises a first antigen-binding site that binds an extracellular part of ERBB2 and a second antigen-binding site that binds an extracellular part of ERBB3. In certain aspects, said bispecific antibody has a first antigen-binding site that binds domain I of ERBB2 and a second antigen-binding site that binds domain III of ERBB3. In certain aspects, the affinity of the first antigen-binding site for ERBB2 is lower than the affinity of the second antigen-binding site for ERBB3. In certain aspects, said bispecific antibody is or comprises zenocutuzumab (International Nonproprietary Name).

In certain aspects, the cancer is not NRG1 fusion positive. Not being NRG1 fusion positive, or NRG1 fusion negative, means that any cancer or subject as mentioned herein, does not comprise an NRG1 fusion gene, in particular said subject or cancer does not comprise an NRG fusion gene which expresses a protein that comprises an NRG1 EGF-like domain. In certain aspects, said subject comprises a cancer-associated cell, such as a cancer-associated fibroblast, which is not NRG1-fusion positive. Said cancer-associated cell is typically located in the human prostate gland.

The bispecific antibody of the present disclosure comprises a first antigen-binding site that binds an extracellular part of ERBB2 and a second antigen-binding site that binds an extracellular part of ERBB3. In certain aspects, said bispecific antibody has a first antigen-binding site that binds domain I of ERBB2 and a second antigen-binding site that binds domain III of ERBB3. In certain aspects, the affinity of the first antigen-binding site for ERBB2 is lower than the affinity of the second antigen-binding site for ERBB3.

In certain aspects, the bispecific antibody comprises
i) at least the CDR1, CDR2 and CDR3 sequences of an ERBB2 specific heavy chain variable region selected from the group consisting of MF2973, MF3004, MF3958, MF2971, MF3025, MF2916, MF3991, MF3031 and MF3003 or wherein said antibody comprises CDR sequences that differ in at most 3 amino acids, preferably in at most 2 amino acids, preferably in at most 1 amino acid from the CDR1, CDR2 and CDR3 sequences of MF2973, MF3004, MF3958, MF2971, MF3025, MF2916, MF3991, MF3031 or MF3003; and/or
ii) at least the CDR1, CDR2 and CDR3 sequences of an ERBB3 specific heavy chain variable region selected from the group consisting of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

In certain aspects, the bispecific antibody comprises
i) an ERBB2 specific heavy chain variable region sequence selected from the group consisting of the heavy chain variable region sequences of MF2973, MF3004, MF3958, MF2971, MF3025, MF2916, MF3991, MF3031 and MF3003, or wherein said antibody comprises a heavy chain variable region sequence that differs in at most 15 amino acids from the heavy chain variable region sequence of MF2973, MF3004, MF3958, MF2971, MF3025, MF2916, MF3991, MF3031 or MF3003; and/or
ii) an ERBB3 specific heavy chain variable region sequence selected from the group consisting of the heavy chain variable region sequences of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises a heavy chain variable region sequence that differs in at most 15 amino acids from the heavy chain variable region sequence of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

In certain aspects, the bispecific antibody for use in the present invention is MF3958×MF3178, which comprises heavy chain variable regions MF3958 (anti-ERBB2) and MF3178 (anti-ERBB3). MF3958×MF3178 has been demonstrated to be well tolerated as a single agent, with low risk for immunogenicity in the treatment of over 100 patients, making it an excellent agent for combination therapy, providing an advantage over other anti-ERBB2 and/or anti-ERBB3 targeting agents. Without being bound to any theory, MF3958×MF3178's efficacy in the treatment of patients having cancer harboring an ERBB2 and ERBB3 positive cell having an ERBB3 mutation is thought to be based on the epitope specificity of MF3958×MF3178 and imbalance of affinity, permitting MF3958×MF3178 to dock onto domain 1 of ERBB2, and block ERBB3 at domain 3 from dimerizing with ERBB2 thereby disrupting activation of the PI3K pathway.

The variable domain that comprises said first antigen binding site and the variable domain that comprises said second antigen binding site of said bispecific antibody preferably comprise a light chain variable region comprising a CDR1 having the sequence (RASQSISSYLN), a CDR2 having the sequence (AASSLQS), and a CDR3 having the sequence (QQSYSTPPT), according to KABAT numbering or according to the IMGT numbering system, the CDRs are QSISSY, AAS and QQSYSTPPT, respectively.

The variable domain that comprises said first antigen binding site and the variable domain that comprises said second antigen binding site of said bispecific antibody preferably comprise a light chain variable region of FIG. 1a or FIG. 1b and/or CDR1, CDR2 and CDR3 sequences QSISSY, AAS and QQSYSTPPT, respectively, according to the IMGT numbering system (FIG. 1f).

The amount of bispecific antibody to be administered to a subject is typically in the therapeutic window, meaning that a sufficient quantity is used for obtaining a therapeutic effect, while the amount does not exceed a threshold value leading to an unacceptable extent of side-effects. The selected dosage level will depend upon a variety of factors including the route of administration, the time of administration, the rate of excretion of the particular compound being employed, the duration of the treatment, other drugs, compounds and/or materials used in combination, the age, sex, weight, condition, general health and prior medical history of the subject being treated, and like factors well known in the medical arts. The dosage is in the range of 200-1000 mg, weekly, biweekly or tri-weekly. Preferably, dosing the therapeutic of the present disclosure, targeting ERBB2×ERBB3, follows a weekly, biweekly or tri-weekly administration regimen of 750 mg, preferably a bi-weekly or tri-weekly dose of 750 mg. The dosing is preferably in subjects with cancer having a solid tumor harboring an ERBB3 mutation, following, a dosing regimen is comprising a weekly flat dose administration of 400 mg, preferably commenced after a single administration of 800 mg. Following this alternative dosing regimen, the bispecific antibody of the invention is preferably administered in a weekly dose of 400 mg for 3 weeks followed by 1 week without dosing. Next, one or more cycles of a period of four weeks, consisting of three weekly flat dosages of 400 mg, followed by a week without administration is followed. This is preferably followed until a therapeutic effect is observed. A dosing regimen of the present disclosure comprises a bi-weekly cycle with a flat dose of 750 mg weekly commenced after an initial administration of a 750 mg infusion over a four-hour period, followed by a biweekly two-hour infusion of 750 mg in a four-week cycle. This is preferably followed until a therapeutic effect is observed.

Dosing preferably involves intravenous injections of two infusions of the bispecific antibody of the invention to arrive at the complete dose, preferably when dosing >360 mg antibody. Alternatively, a single infusion of the complete dose may be given for lower dosages, for instance when dosing ≤360 mg antibody. Pre-medication maybe included in the dosing regimen to mitigate infusion-related reactions.

Preferably, treatment comprises stabilization of the tumor in terms of size or lesions or prevention of further tumor growth, including tumor reduction. Preferably, treatment or administration is with the bispecific antibody according to the invention on a weekly regimen and proceeds for a period of at least 1, 2, 4, 8 or at least 12 months. Preferably, a dosing regimen is followed comprising a weekly cycle with a flat dose of 400 mg weekly commenced after an initial administration of 800 mg. From week 3, the bispecific antibody of the invention is given at a weekly dose of 400 mg for 3 weeks followed by 1 week without dosing of the bispecific antibody of the invention. Alternatively, a dosing regimen is followed comprising a bi-weekly cycle with a flat dose of 750 mg weekly commenced after an initial administration of a 750 mg infusion over a four-hour period, followed by a bi-weekly two-hour infusion of 750 mg in a four-week cycle. A further alternative comprises a tri-weekly administration of a flat dose of 750 mg per subject.

Preferably, a bispecific antibody that has a first antigen-binding site that binds or can bind an extracellular part of ERBB2 and a second antigen-binding site that binds or can bind an extracellular part of ERBB3, in particular MF3958×MF3178, stabilizes tumors of a castration-resistant prostate cancer in terms of size or lesions or the treatment prevents further tumor growth of a castration-resistant prostate cancer.

Antibodies of the present disclosure can be formulated as a pharmaceutical composition comprising a pharmaceutically acceptable carrier, diluent, or excipient, and additional, optional, active agents. The antibodies and compositions comprising the antibodies can be administered by any route including parenteral, enteral, and topical administration. Parenteral administration is usually by injection, and includes, e.g., intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, intratumoral, and intrasternal injection and infusion.

The disclosure provides bispecific antibodies for use in the methods and treatments described herein. Suitable bispecific antibodies comprise a first antigen-binding site that binds or can bind ERBB2 and a second antigen-binding site that binds or can bind ERBB3. The bispecific antibody reduces or can reduce a ligand-induced receptor function of ERBB3 on an ERBB2 and ERBB3 positive cell and/or disrupt ERBB2 and ERBB3 heterodimerization. Preferred antibodies and their preparation are disclosed in WO 2015/130173, which is hereby incorporated by reference. The examples in WO 2015/130173 further describe a number of properties of the antibodies, such as ligand binding and epitope mapping.

In certain aspects, the present disclosure provides an antibody that binds or can bind ERBB3. In certain aspects, said antibody binds domain III of ERBB3 and comprises a CDR1, CDR2 and CDR3 sequence as disclosed herein. In certain aspects, said antibody is a monospecific antibody, such as patritumab (U3-1287/A888), seribantumab (MM-121), lumretuzumab (RG7116, RO-5479599), elgemtumab (LJM716), AV-203, KTN3379 (CDX-3379), or GSK2849330. In certain aspects, said antibody is an antibody-drug conjugate, such as patritumab deruxtecan (U3-1402). In certain aspects, said antibody is a monospecific bivalent antibody.

In certain aspects, patritumab is administered at 18 mg/kg once every 21 days, followed by 9 mg/kg once every 21 days.

In certain aspects, seribantumab is administered at a 40 mg/kg loading dose followed by 20 mg/kg weekly maintenance dose (40/20 mg/kg). Dose or schedule may be adjusted at the discretion of the treating physician. In certain aspects, seribantumab is administered a 3 g weekly dose, administered via intravenous injection (IV).

In certain aspects, lumretuzumab is administered at 500 or 1000 mg every 3 weeks via IV infusion in combination with pertuzumab, wherein pertuzumab is administered as an initial loading dose of 840 mg for every 3 weeks via IV infusion followed by a maintenance dose of 420 mg, every 3 weeks via IV infusion.

In certain aspects, patritumab deruxtecan is administered as an intravenous administration of 5.6 mg/kg every 3 weeks (q3W).

In certain aspects, KTN3379/CDX-3379 is administered as an intravenous administration of 20 mg/kg every 3 weeks (q3W). Alternatively, CDX-3379 is administered at a dose of 12 mg/kg once every 3 weeks in combination with 400 mg/m² cetuximab on the first day followed by weekly doses of 250 mg/m² cetuximab.

In certain aspects, AV-203 is administered as an intravenous administration of 20 mg/kg every 2 weeks (q2W).

In certain aspects, GSK2849330 is administered at a dose of 30 mg/kg once a week.

In certain aspects, bispecific antibodies as disclosed herein comprise:
at least the CDR3 sequence, preferably at least the CDR1, CDR2 and CDR3 sequences, or at least the heavy chain variable region sequence, of an ERBB2 specific heavy chain variable region selected from the group consisting of MF2973, MF3004, MF3958, MF2971, MF3025, MF2916, MF3991, MF3031 and MF3003, or a heavy chain variable region sequence that differs in at most 15 amino acids, preferably in at most 1, 2, 3, 4, 5, 6, 7, 8, 9 or 10 amino acids, more preferably in at most 1, 2, 3, 4 or 5 amino acids, from the recited heavy chain variable region sequences; and/or
at least the CDR3 sequence, preferably at least the CDR1, CDR2 and CDR3 sequences, or at least the heavy chain variable region sequence, of an ERBB 3 specific heavy chain variable region selected from the group consisting of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or a heavy chain variable region sequence that differs in at most 15 amino acids, preferably in at most 1, 2, 3, 4, 5, 6, 7, 8, 9 or 10 amino acids, more preferably in at most 1, 2, 3, 4 or 5 amino acids, from the recited heavy chain variable region sequences.

In certain aspects, antibodies that bind ERBB3, but not necessarily ERBB2, as disclosed herein comprise:
at least the CDR3 sequence, preferably at least the CDR1, CDR2 and CDR3 sequences, or at least the heavy chain variable region sequence, of an ERBB 3 specific heavy chain variable region selected from the group consisting of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or a heavy chain variable region sequence that differs in at most 15 amino acids, preferably in at most 1, 2, 3, 4, 5, 6, 7, 8, 9 or 10 amino acids, more preferably in at most 1, 2, 3, 4 or 5 amino acids, from the recited heavy chain variable region sequences.

CDR sequences are for instance varied for optimization purposes, preferably in order to improve binding efficacy or the stability of the antibody. Optimization is for instance performed by mutagenesis procedures where after the stability and/or binding affinity of the resulting antibodies are preferably tested and an improved ERBB2 or ERBB3-specific CDR sequence is preferably selected. A skilled person is well capable of generating antibody variants comprising at least one altered CDR sequence. For instance, conservative amino acid substitution is applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

In certain aspects, antibodies comprise a variable domain that binds ERBB2, wherein the VH chain of said variable domain comprises the amino acid sequence of VH chain MF2973; MF3004; MF3958 (is humanized MF2971); MF2971; MF3025; MF2916; MF3991 (is humanized MF3004); MF3031 or MF3003; or comprises the amino acid sequence of VH chain MF2973; MF3004; MF3958 (is humanized MF2971); MF2971; MF3025; MF2916; MF3991 (is humanized MF3004); MF3031 or MF3003 as having at most 15, preferably 1, 2, 3, 4, 5, 6, 7, 8, 9 or 10 more preferably at most 1, 2, 3, 4 or 5, amino acid insertions, deletions, substitutions or a combination thereof with respect to the above mentioned VH chain sequence. The VH chain of the variable domain that binds ERBB2 preferably comprises the amino acid sequence of:
MF2971 or a humanized version thereof, wherein said humanized version preferably comprises the amino acid sequence of MF3958; or
MF3004 or a humanized version thereof, wherein said humanized version preferably comprises the amino acid sequence of MF3991. In one embodiment, the VH chain of the variable domain that binds ERBB2 comprises the amino acid sequence of VH chain MF2971 or a humanized version thereof, wherein said humanized version preferably comprises the amino acid sequence of MF3958; or MF3004 or a humanized version thereof, wherein said humanized version preferably comprises the amino acid sequence of MF3991, wherein the recited VH sequences have at most 15, preferably 1, 2, 3, 4, 5, 6, 7, 8, 9 or 10, more preferably at most 1, 2, 3, 4 or 5, amino acid insertions, deletions, substitutions or a combination thereof with respect to the respective sequence. In a preferred embodiment the VH chain of the variable domain that binds ERBB2 comprises the amino acid sequence of MF3958; or comprises the amino acid sequence of MF3958 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence.

The VH chain of the variable domain that binds ERBB3 preferably comprises the amino acid sequence of VH chain MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074; or comprises the amino acid sequence of VH chain MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence. The VH chain of the variable domain that binds ERBB3 preferably comprises the amino acid sequence of MF3178, MF3176, MF3163, MF6058, MF6061 or MF6065; or comprises the amino acid sequence of MF3178, MF3176, MF3163, MF6058, MF6061 or MF6065 having at most 15, preferably 1, 2, 3, 4, 5, 6, 7, 8, 9 or 10, more preferably in at most 1, 2, 3, 4 or 5, amino acid insertions, deletions, substitutions or a combination thereof with respect to the respective VH chain sequence. In a preferred embodiment the VH chain of the variable domain that binds ERBB3 comprises the amino acid sequence of MF3178; or comprises the amino acid sequence of MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence.

Preferably, the above-mentioned amino acid insertions, deletions and substitutions are not present in the CDR3 region. The above-mentioned amino acid insertions, deletions and substitutions are also preferably not present in the CDR1 and CDR2 regions. The above-mentioned amino acid insertions, deletions and substitutions are also preferably not present in the FR4 region.

In certain aspects, the antibody comprises at least the CDR1, CDR2 and CDR3 sequences of MF2971, MF3958, MF3004 or MF3991, most preferably at least the CDR1, CDR2 and CDR3 sequences of MF3958. Said antibody preferably comprises at least the CDR1, CDR2 and CDR3 sequences of MF3178, MF3176, MF3163, MF6058, MF6061 or MF6065, most preferably at least the CDR1, CDR2 and CDR3 sequence of MF3178.

In certain aspects, the ERBB2 specific heavy chain variable region comprises the amino acid sequence of the VH chain MF3958 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH (preferably wherein said insertions, deletions, substitutions are not in CDR1, CDR2, or CDR3). In certain aspects they are also not present in the FR4 region. An amino acid substitution is in certain aspects a conservative amino acid substitution.

In certain aspects, the ERBB3 specific heavy chain variable region comprises the amino acid sequence of the VH chain MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH. The one or more amino acid insertions, deletions, substitutions or a combination thereof are preferably not in the CDR1, CDR2 and CDR3 region of the VH chain. They are also preferably not present in the FR4 region. An amino acid substitution is preferably a conservative amino acid substitution.

In certain aspects, the ERBB2 specific heavy chain variable region comprises the amino acid sequence of the VH chain MF3991 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH (preferably wherein said insertions, deletions, substitutions are not in CDR1, CDR2, or CDR3). They are also preferably not present in the FR4 region. An amino acid substitution is preferably a conservative amino acid substitution.

In certain aspects, the ERBB3 specific heavy chain variable region comprises the amino acid sequence of the VH chain MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH. The one or more amino acid insertions, deletions, substitutions or a combination thereof are preferably not in the CDR1, CDR2 and CDR3 region of the VH chain. They are also preferably not present in the FR4 region. An amino acid substitution is preferably a conservative amino acid substitution.

In certain aspects, the first antigen-binding site of the antibody comprises at least the CDR1, CDR2 and CDR3 sequences of MF3958, or CDR1, CDR2 and CDR3 sequences that differ in at most three, preferably in at most two, preferably in at most one amino acid from the CDR1, CDR2 and CDR3 sequences of MF3958, and wherein said second antigen-binding site comprises at least the CDR1, CDR2 and CDR3 sequence of MF3178, or CDR1, CDR2 and CDR3 sequences that differ in at most three, preferably in at most two, preferably in at most one amino acid from the CDR1, CDR2 and CDR3 sequences of MF3178.

In certain aspects, the bispecific antibody comprises i) a first antigen binding site comprising an ERBB2 specific heavy chain variable region comprising the CDR1, CDR2, and CDR3 sequence of MF3958 and a light chain variable region and ii) a second antigen binding site comprising an ERBB3 specific heavy chain variable region comprising the CDR1, CDR2, and CDR3 sequence of MF3178 and a light chain variable region.

In certain aspects, the ERBB2 specific heavy chain variable region has the MF3958 sequence and the ERBB3 specific heavy chain variable region has the MF3178 sequence. This combination is also referred to as the PB4188 antibody. Preferably, the PB4188 antibody is afucosylated.

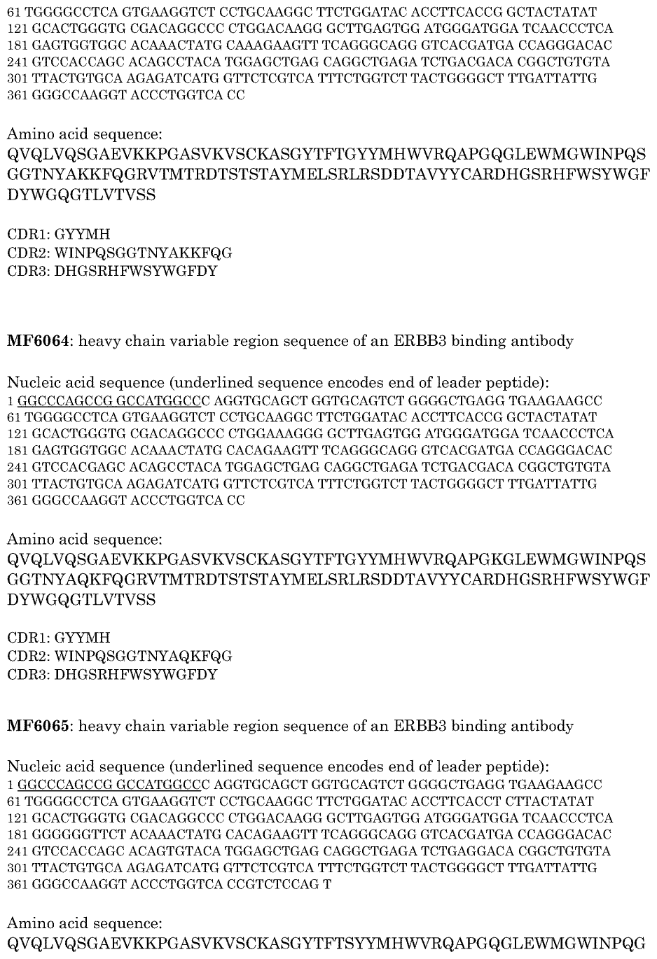

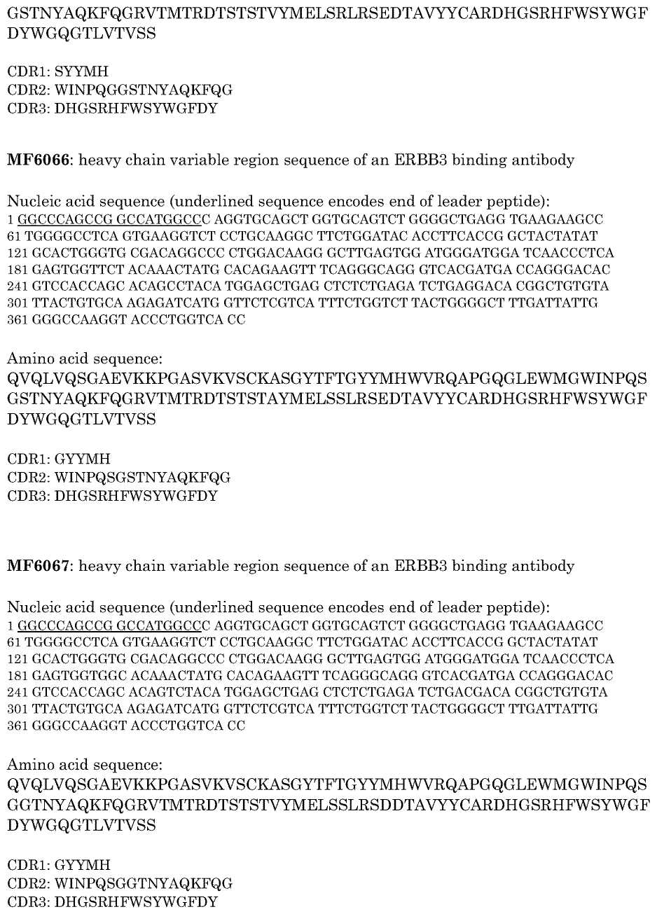

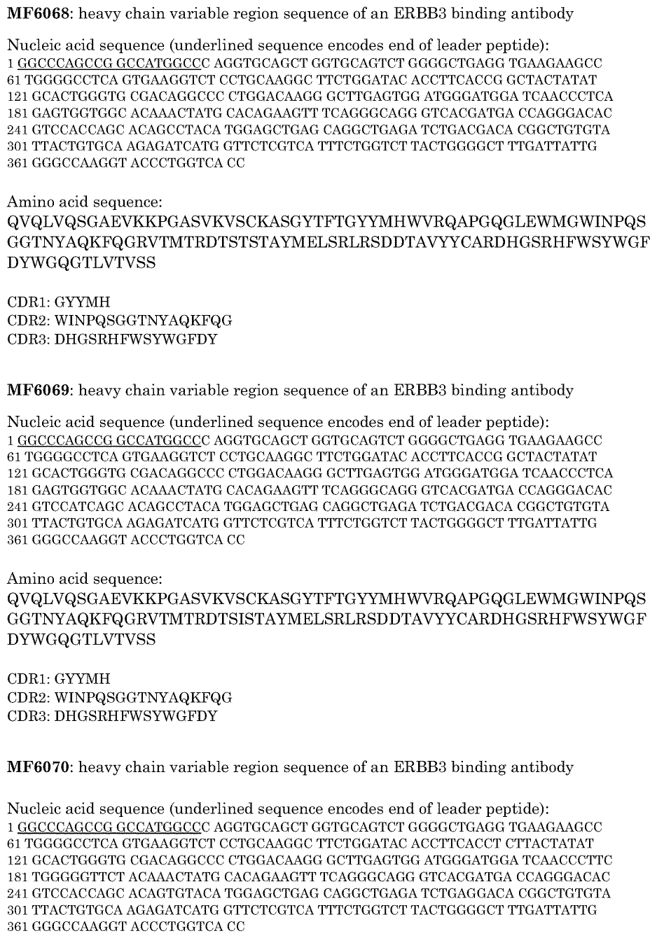

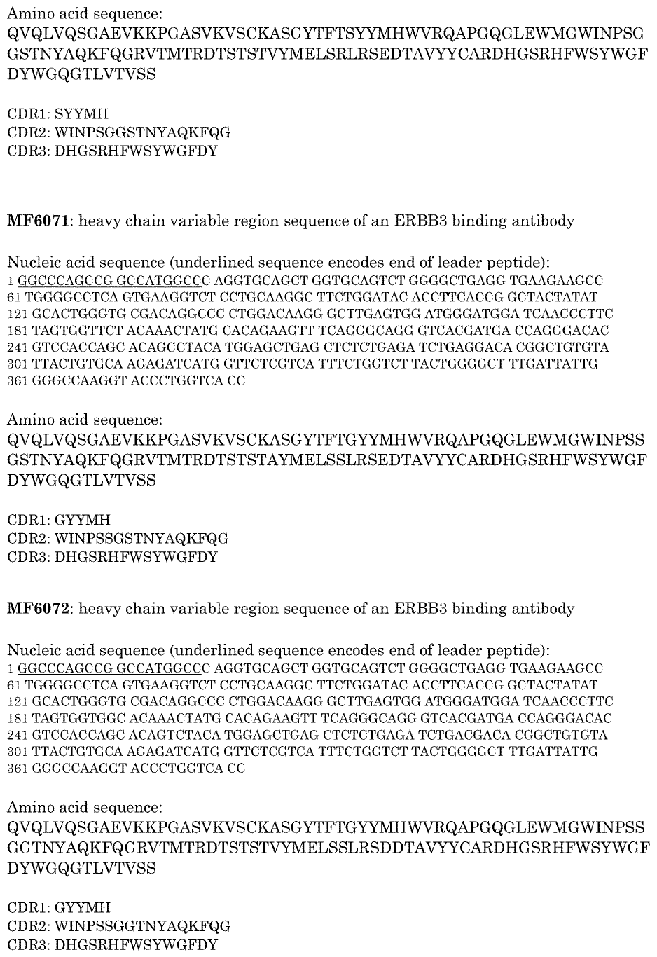

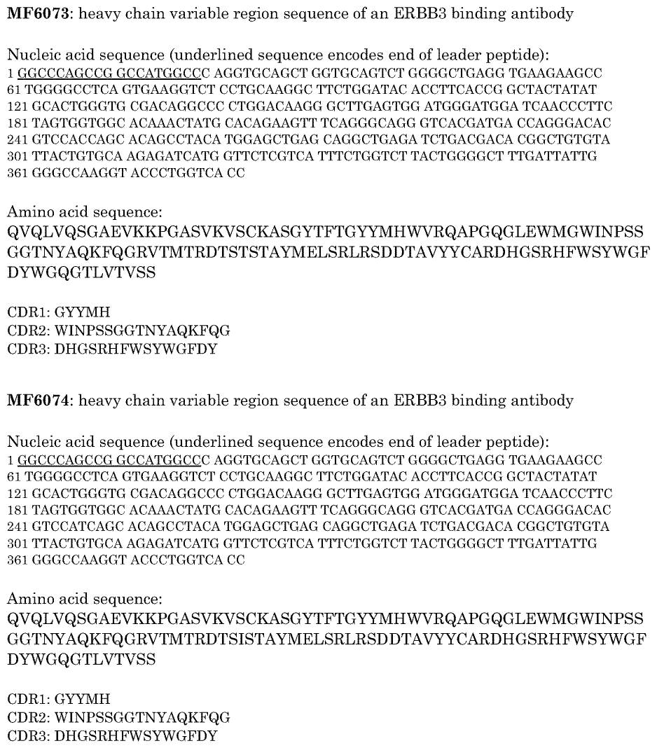

In certain aspects, the bispecific antibody comprises the “heavy chain for ERBB2 binding” as depicted in FIG. 3a and the “heavy chain for ERBB3 binding” as depicted in FIG. 3b.

In certain aspects, the antigen binding sites of the bispecific antibody comprise a common light chain as defined herein, preferably a germline common light chain, preferably the rearranged germline human kappa light chain IgVκ1-39*01/IGJκ1*01 or a fragment or a functional derivative thereof (nomenclature according to the IMGT database worldwide web at imgt.org). The terms rearranged germline human kappa light chain IgVκ1-39*01/IGJκ1*01, IGKV1-39/IGKJ1, huVκ1-39 light chain or in short huVκ1-39 are used. The light chain can have 1, 2, 3, 4 or 5 amino acid insertions, deletions, substitutions or a combination thereof. The mentioned 1, 2, 3, 4 or 5 amino acid substitutions are preferably conservative amino acid substitutions, the insertions, deletions, substitutions or a combination thereof are preferably not in the CDR3 region of the VL chain, preferably not in the CDR1, CDR2 or CDR3 region or FR4 region of the VL chain. Preferably, the first antigen binding site and the second antigen binding site comprise the same light chain variable region, or rather, a common light chain. In certain aspects, the light chain variable region comprises a CDR1 having the sequence (RASQSISSYLN), a CDR2 having the sequence (AASSLQS), and a CDR3 having the sequence (QQSYSTPPT) according to KABAT numbering or according to the IMGT numbering system, the CDRs are QSISSY, AAS and QQSYSTPPT, respectively. Preferably, the light chain variable region comprises the common light chain sequence depicted FIG. 1.

Various methods are available to produce bispecific antibodies and are discussed in WO 2015/130173. One method involves the expression of two different heavy chains and two different light chains in a cell and collecting antibody that is produced by the cell. Antibody produced in this way will typically contain a collection of antibodies with different combinations of heavy and light chains, some of which are the desired bispecific antibody. The bispecific antibody can subsequently be purified from the collection.

The ratio of bispecific to other antibodies that are produced by the cell can be increased in various ways. Preferably, the ratio is increased by expressing not two different light chains but two essentially identical light chains in the cell. This concept is in the art also referred to as the “common light chain” method. When the essentially identically light chains work together with the two different heavy chains allowing the formation of variable domains with different antigen-binding sites and concomitant different binding properties, the ratio of bispecific antibody to other antibody that is produced by the cell is significantly improved over the expression of two different light chains. The ratio of bispecific antibody that is produced by the cell can be further improved by stimulating the pairing of two different heavy chains with each other over the pairing of two identical heavy chains. The art describes various ways in which such heterodimerization of heavy chains can be achieved. A preferred method is described in PCT application No. PCT/NL2013/050294 (WO 2013/157954 A1), which are incorporated herein by reference. Methods and means are disclosed for producing bispecific antibodies from a single cell, whereby means are provided that favor the formation of bispecific antibodies over the formation of monospecific antibodies.

For the purpose of clarity and a concise description features are described herein as part of the same or separate embodiments, however, it will be appreciated that the scope of the invention may include embodiments having combinations of all or some of the features described. Also, any aspect disclosed herein as mentioned to be related to any use in a method of treatment according to the present disclosure applies equally to the method of treatment according to the present disclosure, and vice versa.

### CLAUSES

1. A bispecific antibody that comprises an antigen binding site that can bind an extracellular part of ERBB2 and an antigen binding site that can bind an extracellular part of ERBB3 for use in a method of treatment of a castration-resistant prostate cancer in a subject.

2. The bispecific antibody for use according to clause 1, wherein the cancer has progressed after prior treatment with an androgen receptor axis-targeting agent.

3. The bispecific antibody for use according to clause 2, wherein the androgen receptor axis-targeting agent is an androgen receptor antagonist, such as a second generation androgen receptor antagonist.

4. The bispecific antibody for use according to clause 3, wherein the androgen receptor antagonist is enzalutamide.

5. The bispecific antibody for use according to clause 2, wherein the androgen receptor axis-targeting agent is an androgen synthesis inhibitor, such as abiraterone acetate.

6. The bispecific antibody for use according to any one of the preceding clauses, wherein the method of treatment further comprises the use of an androgen receptor axis-targeting agent.

7. The bispecific antibody for use according to any one of clauses 1-5, wherein the method of treatment further comprises the use of an androgen receptor antagonist, such as a second generation androgen receptor antagonist, such as enzalutamide.

8. The bispecific antibody for use according to any one of clauses 1-5, wherein the method of treatment further comprises the use of an androgen synthesis inhibitor, such as abiraterone acetate.

9. The bispecific antibody for use according to any one of clauses 1˜4 or 6-7, wherein if the cancer has progressed after prior treatment with an androgen receptor antagonist, the method of treatment with the bispecific antibody further comprises the use of an androgen receptor antagonist.

10. The bispecific antibody for use according to clause 9, wherein the androgen receptor antagonist used in the prior treatment and the androgen receptor antagonist further used in the method of treatment with the bispecific antibody, are the same.

11. The bispecific antibody for use according to clause 9 or 10, wherein the androgen receptor antagonist used in the prior treatment and the androgen receptor antagonist further used in the method of treatment with the bispecific antibody, are both enzalutamide.

12. The bispecific antibody for use according to any one of clauses 4, 7 or 11, wherein enzalutamide is administered at 160 mg once per day.

13. The bispecific antibody for use according to any one of clauses 1, 2, 5, 6 or 8, wherein if the cancer has progressed after prior treatment with an androgen synthesis inhibitor, the method of treatment with the bispecific antibody further comprises the use of an androgen synthesis inhibitor.

14. The bispecific antibody for use according to clause 13, wherein the androgen synthesis inhibitor used in the prior treatment and the androgen synthesis inhibitor further used in the method of treatment with the bispecific antibody, are the same.

15. The bispecific antibody for use according to clause 13 or 14, wherein the androgen synthesis inhibitor used in the prior treatment and the androgen synthesis inhibitor further used in the method of treatment with the bispecific antibody, are both abiraterone acetate.

16. The bispecific antibody for use according to any one of clauses 5, 8 or 15, wherein abiraterone acetate is administered at 1000 mg once per day.

17. The bispecific antibody for use according to clause 16, wherein abiraterone acetate is administered in combination with prednisone at 5 mg twice per day.

18. A method of treatment of a subject having a castration-resistant prostate cancer, the method comprising administering to the subject a bispecific antibody that comprises an antigen binding site that can bind an extracellular part of ERBB2 and an antigen binding site that can bind an extracellular part of ERBB3.

19. Use of a bispecific antibody that comprises an antigen binding site that can bind an extracellular part of ERBB2 and an antigen binding site that can bind an extracellular part of ERBB3 for the manufacture of a medicament for the treatment of castration-resistant prostate cancer in a subject.

20. The method of treatment or use according to clause 18 or 19, wherein the cancer has progressed after prior treatment with an androgen receptor axis-targeting agent.

21. The method of treatment or use according to clause 20, wherein the androgen receptor axis-targeting agent is an androgen receptor antagonist, such as a second generation androgen receptor antagonist.

22. The method of treatment or use according to clause 21, wherein the androgen receptor antagonist is enzalutamide.

23. The method of treatment or use according to clause 20, wherein the androgen receptor axis-targeting agent is an androgen synthesis inhibitor, such as abiraterone acetate.

24. The method of treatment or use according to any one of clauses 18-23, wherein the treatment with the bispecific antibody or use for the manufacture of a medicament further comprises the administration or use of an androgen receptor axis-targeting agent.

25. The method of treatment or use according to any one of clauses 18-23, wherein the treatment with the bispecific antibody or use for the manufacture of a medicament further comprises the administration or use of an androgen receptor antagonist, such as a second generation androgen receptor antagonist, such as enzalutamide.

26. The method of treatment or use according to any one of clauses 18-23, wherein the treatment with the bispecific antibody or use for the manufacture of a medicament further comprises the administration or use of an androgen synthesis inhibitor, such as abiraterone acetate.

27. The method of treatment or use according to any one of clauses 18-26, wherein if the cancer has progressed after prior treatment with an androgen receptor antagonist, the method of treatment with the bispecific antibody or the manufacture of a medicament further comprises the administration or use of an androgen receptor antagonist.

28. The method of treatment or use according to clause 27, wherein the androgen receptor antagonist used in the prior treatment and the androgen receptor antagonist further used in the method of treatment with the bispecific antibody or used for the manufacture of a medicament, are the same.

29. The method of treatment or use according to any one of clauses 27 or 28, wherein the androgen receptor antagonist used in the prior treatment and the androgen receptor antagonist further used in the method of treatment with the bispecific antibody or used for the manufacture of a medicament, are both enzalutamide.

30. The method of treatment or use according to any one of clauses 22, 25 or 29, wherein enzalutamide is administered or is to be administered at 160 mg once per day.

31. The method of treatment or use according to any one of clauses 18, 19, 23, 24 or 26, wherein if the cancer has progressed after prior treatment with an androgen synthesis inhibitor, the method of treatment with the bispecific antibody or the manufacture of a medicament further comprises the administration or use of an androgen synthesis inhibitor.

32. The method of treatment or use according to clause 31, wherein the androgen synthesis inhibitor used in the prior treatment and the androgen synthesis inhibitor further used in the method of treatment with the bispecific antibody or used for the manufacture of a medicament, are the same.

33. The method of treatment or use according to clause 31 or 32, wherein the androgen synthesis inhibitor used in the prior treatment and the androgen synthesis inhibitor further used in the method of treatment with the bispecific antibody or used for the manufacture of a medicament, are both abiraterone acetate.

34. The method of treatment or use according to any one of clauses 23, 26 or 33, wherein abiraterone acetate is administered or is to be administered at 1000 mg once per day.

35. The method of treatment or use according to clause 34, wherein abiraterone acetate is administered or is to be administered in combination with prednisone at 5 mg twice per day.

36. The bispecific antibody for use according to any one of clauses 1-17, or the method of treatment or use according to any one of clauses 18-35, wherein the method of treatment comprises administering said bispecific antibody in an amount of 750 mg once every two weeks.

37. The bispecific antibody for use according to any one of clauses 1-17 or 36, or the method of treatment or use according to any one of clauses 18-36, wherein the subject or cancer has a PTEN wildtype status.

38. The bispecific antibody for use according to any one of clauses 1-17 or 36, or the method of treatment or use according to any one of clauses 18-36, wherein the subject or cancer does not exhibit PTEN loss.

39. The bispecific antibody for use according to any one of clauses 1-17 or any one of clauses 36-38, or the method of treatment or use according to any one of clauses 18-38, wherein the method of treatment comprises administering a therapeutically effective amount of the bispecific antibody that comprises an antigen binding site that can bind an extracellular part of ERBB2 and an antigen binding site that can bind an extracellular part of ERBB3 to a subject in need thereof.

40. The bispecific antibody for use according to any of clauses 6-17 or any one of clauses 36-39, or the method of treatment or use according to any one of clauses 24-39, wherein the androgen receptor axis-targeting agent is administered, or is to be administered, according to the medical prescription instructions as established by health care authorities, such as the FDA.

41. The bispecific antibody for use according to any one of clauses 1-17 or any one of clauses 36-40, or the method of treatment or use according to any one of clauses 18-40, wherein the cancer is characterized by histologically confirmed adenocarcinoma of the prostate, in certain aspects without neuroendocrine differentiation or small cell features.

42. The bispecific antibody for use according to any one of clauses 1-17 or any one of clauses 36-41, or the method of treatment or use according to any one of clauses 18-41, wherein the bispecific antibody comprises a first antigen-binding site that binds or can bind domain I of ERBB2 and a second antigen-binding site that binds or can bind domain III of ERBB3.

43. The bispecific antibody for use according to any one of clauses 1-17 or any one of clauses 36-42, or the method of treatment or use according to any one of clauses 18-42, wherein the bispecific antibody comprises
i) at least the CDR1, CDR2 and CDR3 sequences of an ERBB2 specific heavy chain variable region selected from the group consisting of MF2973, MF3004, MF3958, MF2971, MF3025, MF2916, MF3991, MF3031 and MF3003 or wherein said antibody comprises CDR sequences that differ in at most 3 amino acids, preferably in at most 2 amino acids, preferably in at most 1 amino acid from the CDR1, CDR2 and CDR3 sequences of MF2973, MF3004, MF3958, MF2971, MF3025, MF2916, MF3991, MF3031 or MF3003; and/or
ii) at least the CDR1, CDR2 and CDR3 sequences of an ERBB3 specific heavy chain variable region selected from the group consisting of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

44. The bispecific antibody for use according to any one of clauses 1-17 or any one of clauses 36-43, or the method of treatment or use according to any one of clauses 18-43, wherein the bispecific antibody comprises
i) an ERBB2 specific heavy chain variable region sequence selected from the group consisting of the heavy chain variable region sequences of MF2973, MF3004, MF3958, MF2971, MF3025, MF2916, MF3991, MF3031 and MF3003, or wherein said antibody comprises a heavy chain variable region sequence that differs in at most 15 amino acids from the heavy chain variable region sequences of MF2973, MF3004, MF3958, MF2971, MF3025, MF2916, MF3991, MF3031 or MF3003; and/or
ii) an ERBB3 specific heavy chain variable region sequence selected from the group consisting of the heavy chain variable region sequences of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises a heavy chain variable region sequence that differs in at most 15 amino acids from the heavy chain variable region sequences of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

45. The bispecific antibody for use according to any one of clauses 1-17 or any one of clauses 36-44, or the method of treatment or use according to any one of clauses 18-44, wherein the bispecific antibody comprises heavy chain variable regions MF3958 and MF3178.

46. The bispecific antibody for use according to any one of clauses 1-17 or any one of clauses 36-45, or the method of treatment or use according to any one of clauses 18-45, wherein the bispecific antibody comprises a variable domain that comprises said first antigen binding site and a variable domain that comprises said second antigen binding site, and wherein the first and the second antigen binding sites comprise a light chain variable region which comprises a CDR1 which comprises sequence QSISSY, a CDR2 which comprises sequence AAS and a CDR3 which comprises sequence QQSYSTPPT.

47. An antibody that comprises an antigen binding site that can bind an extracellular part of ERBB3 for use in a method of treatment of a castration-resistant prostate cancer in a subject.

48. A method of treatment of a subject having a castration-resistant prostate cancer, the method comprising administering to the subject an effective amount of an antibody that comprises an antigen binding site that can bind an extracellular part of ERBB3.

49. Use of an antibody that comprises an antigen binding site that can bind an extracellular part of ERBB3 for the manufacture of a medicament for the treatment of castration-resistant prostate cancer in a subject.

50. The antibody for use according to clause 47, the method of treatment of clause 48 or the use according to clause 49, wherein the antibody comprises an antigen-binding site that binds domain III of ERBB3.

51. The antibody for use according to clause 47 or 50, the method of treatment of clause 48 or 50, or the use according to clause 49 or 50, wherein the antibody comprises
i) at least the CDR1, CDR2 and CDR3 sequences of an ERBB3 specific heavy chain variable region selected from the group consisting of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

52. The antibody for use according to any one of clauses 47 or 50-51, the method of treatment of any one of clauses 48 or 50-51, or the use according to any one of clauses 49-51, wherein the antibody comprises
i) an ERBB3 specific heavy chain variable region sequence selected from the group consisting of the heavy chain variable region sequences of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises a heavy chain variable region sequence that differs in at most 15 amino acids from the heavy chain variable region sequence of MF3178; MF3176; MF3163; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

53. The antibody for use according to any one of clauses 47 or 50-52, the method of treatment of any one of clauses 48 or 50-52, or the use according to any one of clauses 49-52, wherein the antibody comprises heavy chain variable region MF3178.

54. The antibody for use according to any one of clauses 47 or 50-53, the method of treatment of any one of clauses 48 or 50-53, or the use according to any one of clauses 49-53, wherein the antibody comprises a variable domain that comprises said antigen binding site that can bind an extracellular part of ERBB3, and wherein the antigen binding site comprises a light chain variable region which comprises a CDR1 which comprises sequence QSISSY, a CDR2 which comprises sequence AAS and a CDR3 which comprises sequence QQSYSTPPT.

55. The antibody for use according to any one of clauses 47 or 50-54, the method of treatment of any one of clauses 48 or 50-54, or the use according to any one of clauses 49-54, wherein the antibody is a monospecific antibody that binds ERBB3, such as a monospecific bivalent antibody.

56. The antibody for use according to any one of clauses 47 or 50-55, the method of treatment of any one of clauses 48 or 50-55, or the use according to any one of clauses 49-55, wherein the antibody comprises patritumab (U3-1287/A888), seribantumab (MM-121), lumretuzumab (RG7116, RO-5479599), elgemtumab (LJM716), AV-203, KTN3379/CDX-3379, GSK2849330 or an antibody-drug conjugate, such as patritumab deruxtecan (U3-1402).

57. The antibody for use according to any one of clauses 47 or 50-55, the method of treatment of any one of clauses 48 or 50-55, or the use according to any one of clauses 49-55, wherein the antibody can reduce or reduces a ligand-induced receptor function of ERBB3.

58. The antibody for use according to any one of clauses 47 or 50-55, the method of treatment of any one of clauses 48 or 50-55, or the use according to any one of clauses 49-55, wherein the antibody comprises an antigen binding site that can bind an extracellular part of ERBB3 which blocks both ERBB3 and its ligand heregulin.

59. The antibody for use according to any one of clauses 47 or 50-58, the method of treatment of any one of clauses 48 or 50-58, or the use according to any one of clauses 49-58, wherein the cancer has progressed after prior treatment with an androgen receptor axis-targeting agent.

60. The antibody for use according to clause 59, the method of treatment of clause 59, or the use according to clause 59, wherein the androgen receptor axis-targeting agent is an androgen receptor antagonist, such as a second generation androgen receptor antagonist.

61. The antibody for use according to clause 60, the method of treatment of clause 60, or the use according to clause 60, wherein the androgen receptor antagonist is enzalutamide.

62. The antibody for use according to clause 59, the method of treatment of clause 59, or the use according to clause 59, wherein the androgen receptor axis-targeting agent is an androgen synthesis inhibitor, such as abiraterone acetate.

63. The antibody for use according to any one of clauses 47 or 50-62, the method of treatment of any one of clauses 48 or 50-62, or the use according to any one of clauses 49-62, wherein the method of treatment further comprises the use of an androgen receptor axis-targeting agent.

64. The antibody for use according to any one of clauses 47 or 50-62, the method of treatment of any one of clauses 48 or 50-62, or the use according to any one of clauses 49-62, wherein the method of treatment further comprises the use of an androgen receptor antagonist, such as a second generation androgen receptor antagonist, such as enzalutamide.

65. The antibody for use according to any one of clauses 47 or 50-62, the method of treatment of any one of clauses 48 or 50-62, or the use according to any one of clauses 49-62, wherein the method of treatment further comprises the use of an androgen synthesis inhibitor, such as abiraterone acetate.

66. The antibody for use according to any one of clauses 47, 50-61 or 63-64, the method of treatment of any one of clauses 48, 50-61 or 63-64, or the use according to any one of clauses 49-61 or 63-64, wherein if the cancer has progressed after prior treatment with an androgen receptor antagonist, the method of treatment with the bispecific antibody further comprises the use of an androgen receptor antagonist.

67. The antibody for use according to clause 66, the method of treatment of clause 66, or the use according to clause 66, wherein the androgen receptor antagonist used in the prior treatment and the androgen receptor antagonist further used in the method of treatment with the bispecific antibody, are the same.

68. The antibody for use according to clause 66 or 67, the method of treatment of clause 66 or 67, or the use according to clause 66 or 67, wherein the androgen receptor antagonist used in the prior treatment and the androgen receptor antagonist further used in the method of treatment with the bispecific antibody, are both enzalutamide.

68. The antibody for use according to any one of clauses 61, 64 or 68, the method of treatment of any one of clauses 61, 64 or 68 or the use according to any one of clauses 61, 64 or 68, wherein enzalutamide is administered at 160 mg once per day.

70. The antibody for use according to any one of clauses 47 or 50-59, 62, 63 or 65, the method of treatment of any one of clauses 48 or 50-59, 62, 63 or 65, or the use according to any one of clauses 49-59, 62, 63 or 65, wherein if the cancer has progressed after prior treatment with an androgen synthesis inhibitor, the method of treatment with the bispecific antibody further comprises the use of an androgen synthesis inhibitor.

71. The antibody for use according to clause 70, the method of treatment of clause 70, or the use according to clause 70, wherein the androgen synthesis inhibitor used in the prior treatment and the androgen synthesis inhibitor further used in the method of treatment with the bispecific antibody, are the same.

72. The antibody for use according to clause 70 or 71, the method of treatment of clause 70 or 71, or the use according to clause 70 or 71, wherein the androgen synthesis inhibitor used in the prior treatment and the androgen synthesis inhibitor further used in the method of treatment with the bispecific antibody, are both abiraterone acetate.

73. The antibody for use according to any one of clause 62, 65 or 72, the method of treatment of any one of clause 62, 65 or 72, or the use according to any one of clause 62, 65 or 72, wherein abiraterone acetate is administered at 1000 mg once per day.

74. The antibody for use according to clause 73, the method of treatment of clause 73, or the use according to clause 73, wherein abiraterone acetate is administered in combination with prednisone at 5 mg twice per day.

75. The antibody for use according to any one of clauses 47 or 50-74, the method of treatment of any one of clauses 48 or 50-74, or the use according to any one of clauses 49-74, wherein the method of treatment comprises administering said bispecific antibody in an amount of 750 mg once every two weeks.

76. The antibody for use according to any one of clauses 47 or 50-75, the method of treatment of any one of clauses 48 or 50-75, or the use according to any one of clauses 49-75, wherein the subject or cancer has a PTEN wildtype status.

77. The antibody for use according to any one of clauses 47 or 50-76, the method of treatment of any one of clauses 48 or 50-76, or the use according to any one of clauses 49-76, wherein the subject or cancer does not exhibit PTEN loss.

78. The antibody for use according to any one of clauses 47 or 50-77, the method of treatment of any one of clauses 48 or 50-77, or the use according to any one of clauses 49-77, wherein the method of treatment comprises administering a therapeutically effective amount of the antibody to a subject in need thereof.

79. The antibody for use according to any one of clauses 47 or 50-78, the method of treatment of any one of clauses 48 or 50-78, or the use according to any one of clauses 49-78, wherein the androgen receptor axis-targeting agent is administered, or is to be administered, according to the medical prescription instructions as established by health care authorities, such as the FDA.

80. The antibody for use according to any one of clauses 47 or 50-79, the method of treatment of any one of clauses 48 or 50-79, or the use according to any one of clauses 49-79, wherein the cancer is characterized by histologically confirmed adenocarcinoma of the prostate, in certain aspects without neuroendocrine differentiation or small cell features.

81. A method of selecting a subject having castration-resistant prostate cancer for treatment with a bispecific antibody that comprises an antigen binding site that can bind an extracellular part of ERBB2 and an antigen binding site that can bind an extracellular part of ERBB3, or for treatment with an antibody that comprises an antigen binding site that can bind an extracellular part of ERBB3, the method comprising: a) determining PTEN status in a sample obtained from the subject; and b) selecting the subject for said treatment if the sample does not exhibit PTEN loss.

82. A method of establishing whether a subject having castration-resistant prostate cancer is likely to respond to treatment with a bispecific antibody that comprises an antigen binding site that can bind an extracellular part of ERBB2 and an antigen binding site that can bind an extracellular part of ERBB3, or to treatment with an antibody that comprises an antigen binding site that can bind an extracellular part of ERBB3, the method comprising: a) determining PTEN status in a sample obtained from the subject; and b) selecting a sample for not exhibiting PTEN loss, thereby establishing that the subject from which the sample is derived is likely to respond to said treatment.

83. A method of classifying a subject having castration-resistant prostate cancer on the basis of PTEN status prior to treatment with a bispecific antibody that comprises an antigen binding site that can bind an extracellular part of ERBB2 and an antigen binding site that can bind an extracellular part of ERBB3, or prior to treatment with an antibody that comprises an antigen binding site that can bind an extracellular part of ERBB3, the method comprising: a) determining PTEN status in a sample obtained from the subject; and b) classifying the subject from which or from whom the sample was obtained as eligible for said treatment if the sample does not exhibit PTEN loss.

84. A method of any one of clauses 81-83, wherein PTEN status is determined using IHC.

85. A method of any one of clauses 81-83, wherein PTEN status is determined using a liquid biopsy.

86. A kit-of-parts comprising a bispecific antibody that comprises an antigen binding site that can bind an extracellular part of ERBB2 and an antigen binding site that can bind an extracellular part of ERBB3 and an androgen receptor axis-targeting agent.

87. The kit-of-parts of clause 86, further comprising instructions for use.

88. The kit-of-parts of clause 86 or 87, wherein the instructions for use include instructions for administration of a bispecific antibody and of an androgen receptor axis-targeting agent.

89. The kit-of-parts of any one of clauses 86-88, wherein the androgen receptor axis-targeting agent comprises an androgen receptor antagonist, such as a second generation androgen receptor antagonist, such as enzalutamide or comprises an androgen synthesis inhibitor, such as abiraterone acetate, and wherein the bispecific antibody comprises zenocutuzumab.

90. The kit-of-parts of clause 89, wherein the instructions for use include the administration of abiraterone acetate at 1000 mg once per day in combination with prednisone at 5 mg twice per day and administration of the bispecific antibody in an amount of 750 mg once every two weeks.

91. The kit-of-parts of clause 89, wherein the instructions for use include the administration of enzalutamide at 160 mg once per day and administration of the bispecific antibody in an amount of 750 mg once every two weeks.

92. The kit-of-parts of clause 90 or 91, wherein the instructions for use of abiraterone acetate or enzalutamide include instructions for oral administration, and administration of the bispecific antibody include instructions for an intravenous injection.

93. A kit-of-parts comprising an antibody that comprises an antigen binding site that can bind an extracellular part of ERBB3 and an androgen receptor axis-targeting agent.

94. The kit-of-parts of clause 93, further comprising instructions for use.

95. The kit-of-parts of clause 93 or 94, wherein the instructions for use include instructions for administration of an antibody and of an androgen receptor axis-targeting agent.

96. The kit-of-parts of any one of clauses 93-95, wherein the androgen receptor axis-targeting agent comprises an androgen receptor antagonist, such as a second generation androgen receptor antagonist, such as enzalutamide or comprises an androgen synthesis inhibitor, such as abiraterone acetate, and wherein the antibody comprises zenocutuzumab.

97. The kit-of-parts of clause 96, wherein the instructions for use include the administration of abiraterone acetate at 1000 mg once per day in combination with prednisone at 5 mg twice per day and administration of the antibody in an amount of 750 mg once every two weeks.

98. The kit-of-parts of clause 96, wherein the instructions for use include the administration of enzalutamide at 160 mg once per day and administration of the antibody in an amount of 750 mg once every two weeks.

99. The kit-of-parts of clause 97 or 98, wherein the instructions for use of abiraterone acetate or enzalutamide include instructions for oral administration, and administration of the antibody include instructions for an intravenous injection.

100. The bispecific antibody for use according to any one of clauses 1-17 or 36, or the method of treatment or use according to any one of clauses 18-36, wherein the subject or cancer has a PTEN loss status.

101. The bispecific antibody for use according to any one of clauses 1-17 or 36, or the method of treatment or use according to any one of clauses 18-36, wherein the subject or cancer exhibits PTEN loss.

102. The bispecific antibody for use according to any one of clauses 1-17 or 36, or the method of treatment or use according to any one of clauses 18-36, wherein the subject or cancer does not have an oncogenic driver mutation in any of the PI3K, AKT, and/or mTOR pathways, or wherein the subject or cancer does not have an upregulation of any of said pathways.

103. The bispecific antibody for use according to any one of clauses 1-17 or 36, or the method of treatment or use according to any one of clauses 18-36, wherein the subject or cancer does not exhibit an oncogenic driver mutation in any of the PI3K, AKT, and/or mTOR pathways, or wherein the subject or cancer does not exhibit an upregulation of any of said pathways.

104. The bispecific antibody for use according to any one of clauses 1-17 or 36, or the method of treatment or use according to any one of clauses 18-36, wherein the subject or cancer does not have an oncogenic driver mutation in any known tumor-associated genes or the proteins encoded therefrom, such as EGFR, cMET, ALK, BRAF, KRAS, NRAS, RET and ROS1.

105. The bispecific antibody for use according to any one of clauses 1-17 or 36, or the method of treatment or use according to any one of clauses 18-36, wherein the subject or cancer does not exhibit an oncogenic driver mutation in any known tumor-associated genes or the proteins encoded therefrom, such as EGFR, cMET, ALK, BRAF, KRAS, NRAS, RET and ROS1.

106. The antibody for use according to any one of clauses 47 or 50-75, the method of treatment of any one of clauses 48 or 50-75, or the use according to any one of clauses 49-75, wherein the subject or cancer has a PTEN loss status.

107. The antibody for use according to any one of clauses 47 or 50-75, the method of treatment of any one of clauses 48 or 50-75, or the use according to any one of clauses 49-75, wherein the subject or cancer exhibits PTEN loss.

108. The antibody for use according to any one of clauses 47 or 50-75, the method of treatment of any one of clauses 48 or 50-75, or the use according to any one of clauses 49-75, wherein the subject or cancer does not have an oncogenic driver mutation in any known tumor-associated genes or the proteins encoded therefrom, such as EGFR, cMET, ALK, BRAF, KRAS, NRAS, RET and ROS1.

109. The antibody for use according to any one of clauses 47 or 50-75, the method of treatment of any one of clauses 48 or 50-75, or the use according to any one of clauses 49-75, wherein the subject or cancer does not exhibit an oncogenic driver mutation in any known tumor-associated genes or the proteins encoded therefrom, such as EGFR, cMET, ALK, BRAF, KRAS, NRAS, RET and ROS1.

110. A method of selecting a subject having castration-resistant prostate cancer for treatment with a bispecific antibody that comprises an antigen binding site that can bind an extracellular part of ERBB2 and an antigen binding site that can bind an extracellular part of ERBB3, or for treatment with an antibody that comprises an antigen binding site that can bind an extracellular part of ERBB3, the method comprising: a) determining the existence of an oncogenic driver mutation in any one of the PI3K, AKT and/or mTOR pathways, or a mutation that modulates the PI3K, AKT and/or mTOR pathways, in a sample obtained from the cancer of said subject; and b) selecting said subject for said treatment if the sample lacks the existence of an oncogenic driver mutation in the PI3K, AKT, and/or mTOR pathways, or a mutation that modulates the PI3K, AKT, and/or mTOR pathways.

111. The method of clause 110, wherein the subject's cancer is tested by next generation sequencing, such as DNA, RNA or whole transcriptome sequencing.

112. A method of selecting a subject having castration-resistant prostate cancer for treatment with a bispecific antibody that comprises an antigen binding site that can bind an extracellular part of ERBB2 and an antigen binding site that can bind an extracellular part of ERBB3, or for treatment with an antibody that comprises an antigen binding site that can bind an extracellular part of ERBB3, the method comprising: a) determining the existence of an oncogenic driver mutation in any one of EGFR, cMET, ALK, BRAF, KRAS, NRAS, RET and ROS1, in a sample obtained from the cancer of said subject; and b) selecting said subject for said treatment if the sample lacks the existence of said oncogenic driver mutation.

113. The method of clause 110, wherein the subject's cancer is tested by next generation sequencing, such as DNA, RNA or whole transcriptome sequencing.

114. A method of selecting a subject having castration-resistant prostate cancer for treatment with a bispecific antibody that comprises an antigen binding site that can bind an extracellular part of ERBB2 and an antigen binding site that can bind an extracellular part of ERBB3, or for treatment with an antibody that comprises an antigen binding site that can bind an extracellular part of ERBB3, the method comprising: a) testing the subject's cancer by next generation sequencing for the existence of an oncogenic driver mutation that modulates any one of the PI3K, AKT and/or mTOR pathway, in a sample obtained from the subject; and b) classifying the subject from which, or from whom, the sample was obtained as eligible for said treatment if said sample lacks the existence of said oncogenic driver mutation.

115. A method of selecting a subject having castration-resistant prostate cancer for treatment with a bispecific antibody that comprises an antigen binding site that can bind an extracellular part of ERBB2 and an antigen binding site that can bind an extracellular part of ERBB3, or for treatment with an antibody that comprises an antigen binding site that can bind an extracellular part of ERBB3, the method comprising: a) testing the subject's cancer by next generation sequencing for the existence of an oncogenic driver mutation, in certain aspects an oncogenic driver mutation in any one of EGFR, cMET, ALK, BRAF, KRAS, NRAS, RET and ROS1, in a sample obtained from the subject; and b) classifying the subject from which, or from whom, the sample was obtained as eligible for said treatment if said sample lacks the existence of said oncogenic driver mutation.

116. The method of any one of clauses 110-115, wherein the subject or cancer furthermore has PTEN wildtype status.

### EXAMPLES

As used herein “MFXXXX” wherein X is independently a numeral 0-9, refers to a Fab comprising a variable domain wherein the VH has the amino acid sequence identified by the 4 digits depicted in FIG. 3. Unless otherwise indicated the light chain variable region typically has a sequence of FIG. 1b. The light chain constant region in the examples has a sequence as depicted in FIG. 1c. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as WO2013/157954) and FIG. 2d and FIG. 2e. Bispecific antibodies in the examples have an Fc tail with a KK/DE CH3 heterodimerization domain, a CH2 domain and a CH1 domain as indicated in FIG. 2, a common light chain as indicated in FIG. 1a and a VHs as specified by the MF numbers.

### Example 1: Multispecific Antibodies Binding Domain I of ERBB2 and Domain III of ERBB3

Bispecific antibodies comprising heavy chain variable regions as mentioned in Table 2 and 3 were obtained as described in WO2015/130173. The indicated ERBB3 binding domains may also be used to provide monospecific antibodies.

TABLE 2
SEQ ID NOs for heavy chain variable regions of
bispecific antibodies binding ERBB2 and ERBB3.

| ERBB2 | | ERBB3 | |
|---|---|---|---|
| binding | | binding | |
| domain ID | SEQ ID NOs | domain ID | SEQ ID NOs |
| MF2973 | SEQ ID NO: 2 | MF3178 | SEQ ID NO: 47 |
| MF3004 | SEQ ID NO: 7 | MF3176 | SEQ ID NO: 52 |
| MF3958 | SEQ ID NO: 12 | MF3163 | SEQ ID NO: 57 |
| MF2971 | SEQ ID NO: 17 | MF6055 | SEQ ID NO: 62 |
| MF3025 | SEQ ID NO: 22 | MF6056 | SEQ ID NO: 67 |
| MF2916 | SEQ ID NO: 27 | MF6057 | SEQ ID NO: 72 |
| MF3991 | SEQ ID NO: 32 | MF6058 | SEQ ID NO: 77 |
| MF3031 | SEQ ID NO: 37 | MF6059 | SEQ ID NO: 82 |
| MF3003 | SEQ ID NO: 42 | MF6060 | SEQ ID NO: 87 |
| | | MF6061 | SEQ ID NO: 92 |
| | | MF6062 | SEQ ID NO: 97 |
| | | MF6063 | SEQ ID NO: 102 |
| | | MF6064 | SEQ ID NO: 107 |
| | | MF6065 | SEQ ID NO: 112 |
| | | MF6066 | SEQ ID NO: 117 |
| | | MF6067 | SEQ ID NO: 122 |
| | | MF6068 | SEQ ID NO: 127 |
| | | MF6069 | SEQ ID NO: 132 |
| | | MF6070 | SEQ ID NO: 137 |
| | | MF6071 | SEQ ID NO: 142 |
| | | MF6072 | SEQ ID NO: 147 |
| | | MF6073 | SEQ ID NO: 152 |
| | | MF6074 | SEQ ID NO: 157 |

TABLE 3
Any of the heavy chain variable regions binding ERBB2 can be combined with any of the heavy chain variable
regions binding ERBB3 in the bispecific antibodies of the present disclosure. Variants of the variable
heavy chain regions may also be combined in the bispecific antibodies of the present disclosure.

| | MF2973 | MF3004 | MF3958 | MF2971 | MF3025 | MF2916 | MF3991 | MF3031 | MF3003 |
|---|---|---|---|---|---|---|---|---|---|
| MF3178 | X | X | X | X | X | X | X | X | X |
| MF3176 | X | X | X | X | X | X | X | X | X |
| MF3163 | X | X | X | X | X | X | X | X | X |
| MF6055 | X | X | X | X | X | X | X | X | X |
| MF6056 | X | X | X | X | X | X | X | X | X |
| MF6057 | X | X | X | X | X | X | X | X | X |
| MF6058 | X | X | X | X | X | X | X | X | X |
| MF6059 | X | X | X | X | X | X | X | X | X |
| MF6060 | X | X | X | X | X | X | X | X | X |
| MF6061 | X | X | X | X | X | X | X | X | X |
| MF6062 | X | X | X | X | X | X | X | X | X |
| MF6063 | X | X | X | X | X | X | X | X | X |
| MF6064 | X | X | X | X | X | X | X | X | X |
| MF6065 | X | X | X | X | X | X | X | X | X |
| MF6066 | X | X | X | X | X | X | X | X | X |
| MF6067 | X | X | X | X | X | X | X | X | X |
| MF6068 | X | X | X | X | X | X | X | X | X |
| MF6069 | X | X | X | X | X | X | X | X | X |
| MF6070 | X | X | X | X | X | X | X | X | X |
| MF6071 | X | X | X | X | X | X | X | X | X |
| MF6072 | X | X | X | X | X | X | X | X | X |
| MF6073 | X | X | X | X | X | X | X | X | X |
| MF6074 | X | X | X | X | X | X | X | X | X |

### Example 2

a Clinical Study with Zenocutuzumab, a Full Length IgG1 Bispecific Antibody Comprising MF3958×MF3178, Targeting ERBB2 and ERBB3, in Patients with Castration-Resistant Prostate Cancer.

This is a Phase II, open-label, multicenter, international study designed to evaluate the efficacy of zenocutuzumab in patients with metastatic mCRPC with evidence of disease progression in accordance with Prostate Cancer Clinical Trials Working Group 3 (PCWG3) criteria on the last line of prior hormonal therapy that included either the second-generation AR antagonist enzalutamide or the androgen synthesis inhibitor abiraterone acetate. Patients will be enrolled and will receive zenocutuzumab in combination with the AR targeting agent on which they experienced disease progression immediately before study entry. A cohort of patients with phosphate and tensin homolog (PTEN) wild-type status will be enrolled. A safety run-in will be performed in the first 4 to 6 patients.

### Study Medication

Zenocutuzumab is an investigational medicinal product and the companion AR signaling inhibitors are considered non-investigational medicinal products. Zenocutuzumab, a bispecific humanized full length IgG1 antibody, is formulated at 20 mg/mL.

### Zenocutuzumab

Zenocutuzumab will be administered as a 2-hour IV infusion on Day 1, then Q2W of every 28-day cycle. A flat dose of 750 mg will be administered.

Patients will receive zenocutuzumab in combination with other medications. Before each zenocutuzumab infusion, premedication is required. The mandatory premedication regimen is:
Paracetamol/acetaminophen 1000 mg PO or IV.
Dexchlorpheniramine 5 mg IV (or other anti-H1 equivalent, PO or IV).
Dexamethasone 10 mg IV (or equivalent, PO or IV). Corticosteroids are only mandatory before Cycle 1 Day 1 administration and should be used for subsequent injections at the Investigator's discretion to manage infusion-related reactions (IRRs).
The use of an H2 antagonist is optional and can be given at the Investigator's discretion.

### Next-Generation AR Signaling Inhibitors

Patients will receive zenocutuzumab in combination with one of the following next generation AR signaling inhibitors that the patient was receiving immediately prior to entering the study:
Abiraterone acetate (ZYTIGA®) 1000 mg PO QD in combination with prednisone 5 mg PO twice daily (BID).
Enzalutamide (XTANDIR) 160 mg PO QD.

Administration of enzalutamide or abiraterone acetate will be initiated on Cycle 1 Day 1.

Abiraterone acetate: Abiraterone acetate is taken in combination with prednisone and should be taken on an empty stomach. No food should be consumed for at least 2 hours before and for at least 1 hour after each dose is taken. Tablets should be swallowed whole with water. Abiraterone acetate is available as a 250-mg tablet, which is white to off white, oval shaped and debossed with AA250 on one side. Tablets should be stored at 20° C. to 25° C. (68° F.-77° F.); temperature excursions to 15° C. to 30° C. (59° F.-86° F.) are permitted.

Enzalutamide: Enzalutamide may be taken with or without food. Capsules should be swallowed whole. Enzalutamide is supplied as a 40 mg white to off-white oblong soft gelatin capsule imprinted in black ink with “MDV.” Capsules should be stored at 20° C. to 25° C. (68° F.-77° F.) in a dry place with the container tightly closed; temperature excursions are permitted to 15° C. to 30° C. (59° F.-86° F.).

### Treatment Adaptation

Next-generation AR signaling inhibitors will be administered according to the local prescribing information for each drug. Dose adjustment of enzalutamide or abiraterone acetate is permitted based on clinical judgement for drug related toxicities: Abiraterone acetate: For a patient who develops hepatotoxicity during treatment (ALT and/or AST greater than 5×ULN or total bilirubin greater than 3×ULN), treatment should be interrupted. Treatment may be restarted at a reduced dose of 750 mg QD once liver function tests have returned to the patient's baseline or to ALT and AST values ≤2.5×ULN and total bilirubin less ≤1.5×ULN. Once the patient resumes treatment, serum transaminases and bilirubin should be monitored at a minimum of every 2 weeks for 3 months and monthly thereafter. If hepatotoxicity recurs at the dose of 750 mg QD, re-treatment may be restarted at a reduced dose of 500 mg QD once function tests have returned to the patient's baseline or to ALT and AST less ≤2.5×ULN and total bilirubin ≤1.5×ULN. If hepatotoxicity recurs at the reduced dose of 500 mg QD, treatment should be discontinued.

Enzalutamide: If a patient experiences a Grade ≥3 toxicity or an intolerable side effect, withhold dosing for 1 week or until symptoms improve to Grade ≤2, then resume at the same or a reduced dose (120 or 80 mg), if warranted.

### Treatment Withdrawal

Zenocutuzumab will be administered until any of the following occurs, at which point it will be definitively discontinued, except in cases of perceived benefit and in agreement with the Sponsor. Continuation of single-agent treatment in the event of benefit will be discussed on a case-by-case basis and in agreement with the Sponsor.
Progressive disease.
AEs/unacceptable toxicity.
Withdrawal of consent.
Patient noncompliance.
Investigator decision (eg, clinical deterioration).
Treatment interruption >6 consecutive weeks.
Discontinuation of any treatment combination drug.

Eligible patients will be enrolled and will receive sequential treatment cycles, with a treatment cycle being 4 weeks (28 days). All patients will receive zenocutuzumab with a flat dose of 750 mg IV Q2W.

### Study Population

### Inclusion Criteria

Patients must fulfill all of the following requirements to enter the study:
1. Signed informed consent before initiation of any study procedures.
2. Age ≥18 years at signature of informed consent.
3. Eastern Cooperative Oncology Group (ECOG) performance status of 0 or 1.
4. Estimated life expectancy of ≥12 weeks.
5. Minimum of 3 weeks since any major surgery, completion of radiation, completion of all prior systemic anticancer therapy, or at least 5-half-lives if the prior therapy is a single agent small molecule therapeutic, and adequately recovered from the acute toxicities of any prior therapy to Grade ≤1 National Cancer Institute (NCI)-Common Terminology Criteria for AEs (CTCAE) v. 5.0, except alopecia or neuropathy.
6. Left ventricular ejection fraction (LVEF) ≥50% by echocardiogram (ECHO) or multigated acquisition scan (MUGA).
7. Adequate organ function:

$$Absolute neutrophil count \geq 1.5 \times {10}^{9} / L .$$

$$Hemoglobin \geq 9 g / dL .$$

$$Platelets \geq 100 \times {10}^{9} / L .$$

Serum calcium within normal ranges (or corrected with supplements).
Alanine aminotransferase (ALT), aspartate aminotransferase (AST)≤2.5×upper limit of normal (ULN) (in case of liver involvement by malignancy, ALT/AST≤5×ULN will be allowed).
Total bilirubin≤1.5×ULN (in case of Gilbert disease, total bilirubin≤3×ULN will be allowed).
Estimated glomerular filtration rate of >30 mL/min based on the Cockroft-Gault formula.

$$Serum albumin > 3. g / dL .$$

8. Availability of a representative tumor specimen, either a formalin-fixed paraffin embedded (FFPE) de novo (ie, obtained up to 2 months before signing of the informed consent form or an FFPE archival tumor sample, preferably collected within 2 years of the start of study treatment. A fresh FFPE sample is preferred.

9. Sexually active male and female patients of childbearing potential must agree to use 1 of the following highly effective methods of birth control during the entire duration of the study and for 6 months after final administration of zenocutuzumab:
Combined (estrogen- and progestogen-containing) hormonal contraception associated with inhibition of ovulation: Oral, Intravaginal, Transdermal.
Progestogen-only hormonal contraception associated with inhibition of ovulation: Oral, Injectable, Implantable, Intrauterine device, Intrauterine hormone-releasing system, Bilateral tubal occlusion, Vasectomized partner, Sexual abstinence.

Further inclusion criteria: Patients must fulfill all of the following requirements to enter the study:
B1. Histologically confirmed adenocarcinoma of the prostate without neuroendocrine differentiation or small cell features.
B2. Metastatic disease documented by at least 2 bone lesions on whole body bone scintigraphy, or soft tissue disease documented by computed tomography (CT) scan/magnetic resonance imaging (MRI).
B3. Ongoing androgen deprivation with a serum testosterone level≤1.73 nmol/L (≤50 ng/dL) at screening, and prior to start of treatment.
B4. Current ongoing therapy with a next-generation AR signaling inhibitor (enzalutamide or abiraterone) started at least 90 days before screening, and prior to start of treatment. An interruption of dosing of a maximum of 30 days is permitted before resuming the agent.
B5. Progressive disease by PCWG3 criteria for study entry defined as 1 of the following:
   Prostate-specific antigen (PSA) progression defined as 2 increases in PSA over a previous reference value reaching a minimum value of >1 ng/mL at screening, and prior to start of treatment (if the patient had an interruption of dosing of abiraterone or enzalutamide of >14 days, rising PSA must be documented after resuming drug).
   Soft tissue disease progression defined by RECIST v1.1.
   Previously normal (<10 mm) lymph nodes must have grown by >5 mm in the short axis to be considered to have progressed.
   Bone disease progression defined by >2 new lesions on whole body bone scintigraphy.
B6. Patients receiving bisphosphonates or denosumab for bone health must have been on a stable dose for at least 4 weeks before study treatment initiation.
B7. Able to swallow oral medications and absence of gastrointestinal conditions (eg, malabsorption, resection) deemed to jeopardize intestinal absorption.

### Exclusion Criteria

Exclusion Criteria are the presence of any of the following criteria excludes a patient from participating in the study:
1. Central nervous system metastases that are untreated or symptomatic, or require radiation, surgery, or continued steroid therapy to control symptoms within 14 days of study entry.
2. Previous exposure to anti-ERBB3-directed therapies.
3. Known leptomeningeal involvement.
4. Participation in another interventional clinical trial or treatment with any investigational drug within 4 weeks before study entry.
5. Chronic use of high-dose oral corticosteroid therapy (>10 mg of prednisone-equivalent a day).
6. Uncontrolled hypertension (systolic blood pressure >150 mmHg and/or diastolic blood pressure >100 mmHg) or unstable angina.
7. History of congestive heart failure Class II-IV by New York Heart Association criteria, or serious cardiac arrhythmia requiring treatment (except atrial fibrillation, or paroxysmal supraventricular tachycardia).
8. History of myocardial infarction within 6 months of study entry.
9. History of prior or concomitant malignancies (other than excised nonmelanoma skin cancer, cured in situ cervical carcinoma, or low-grade Ta or T1 urothelial carcinoma of the bladder that has undergone potentially curative therapy) within 3 years of study entry.
10. Current serious illness or medical conditions including, but not limited to uncontrolled active infection, and clinically significant pulmonary, metabolic, or psychiatric disorders.
11. Patients with the following known infectious diseases:
   Known active hepatitis B infection (hepatitis B surface antigen [HBsAg] positive) without receiving antiviral treatment. Note: Patients with active hepatitis B (HBsAg positive) must receive antiviral treatment with lamivudine, tenofovir, entecavir, or other antiviral agents, starting at least >7 days before the initiation of the study treatment. Patients with antecedents of hepatitis B (anti-HBc positive, HBsAg, and hepatitis B virus [HBV] DNA negative) are eligible.
12. Known human immunodeficiency virus (HIV)-positive patients unless the CD4+ count is ≥300/μL, viral load is undetectable, and the patient is currently receiving highly active antiretroviral therapy.

Further exclusion criteria which excludes a patient from participating in the study are the presence of any of the following criteria:
13. More than 2 lines of a second-generation hormonal agent for metastatic disease.
14. More than 2 lines of systemic chemotherapy for metastatic disease.
15. Patients with only nonmeasurable lesions other than bone metastasis (eg, pleural effusion, ascites, other visceral locations).
16. A history of seizure or any condition predisposing patient to seizure within 12 months before study treatment, including history of unexplained loss of consciousness or transient ischemic attack, for patients receiving enzalutamide.

### Management of Infusion-Related Reactions

Patients will be monitored closely during the study treatment period. Patients must be premedicated with antihistamines, paracetamol/acetaminophen, and corticosteroids before each infusion of zenocutuzumab.

### Prophylactic and Concomitant Medications

### Permitted Medications

All medication necessary for the wellbeing of the patient and which is not expected to interfere with evaluation of the study drug, including supportive treatment of symptoms and AEs or standard treatment of concomitant conditions, may be given at the Investigator's discretion.

Luteinizing hormone-releasing hormone (LHRH) agonists or antagonists in patients who did not previously undergo bilateral orchiectomy are permitted.

### Prohibited Medications

Concomitant chronic oral corticosteroids (>10 mg/day prednisone equivalent), tumor necrosis factor (TNF)-α inhibitors, anti-T-cell antibodies (due to risk of immunosuppression).
Any investigational drug during the study or for 4 weeks before the first dose of study treatment.
It is preferred patients refrain from beginning a new investigational drug for at least 4 weeks after the last dose of study drug.
Systemic anticancer therapy. Note: For patients in Group B, the next generation AR signaling inhibitors that the patient was receiving immediately prior to entering the study is permitted.

### Efficacy Assessments: Measures of Response, Tumor Measurements

Radiological measurement of tumors to assess antitumor effect of zenocutuzumab will be performed by CT scan or MRI. Imaging will be performed according to standard local practice and data will be collected in the electronic case report form. Imaging of all patients will be assessed by the local investigator.

Brain MRI or CT scans should be performed at the same frequency as CT/MRI of chest, abdomen, and pelvis, and only if brain metastases are detected on the Screening scan. Additional imaging of anatomical sites (eg, head, neck) should be performed as applicable for the patient's tumor type. Note that where there is a rationale for assessment of bone lesions, these assessments will be performed as part of the CT or MRI assessment and will not require additional radiological bone scan assessment.

Additional scans may be performed to confirm a response as appropriate. Any requirement for confirmatory scans will typically be performed at the next protocol-required assessment time point or at the Follow-Up Visit. For the first tumor assessment only, a window of +3 days is allowed. Thereafter a ±3-day window is permitted but should precede initiation of the next treatment cycle. Patients who discontinue treatment for reasons other than disease progression and who do not withdraw consent will have their disease status assessed every 8 weeks for up to 12 months until disease progression and/or until the initiation of new anticancer treatment or withdrawal of consent, whichever occurs first.

Whole body bone scintigraphy will be performed every 8 weeks for the first 12 months of treatment and every 12 weeks thereafter. Tumor assessments will be completed according to PCWG3-modified RECIST criteria.

### Tumor Markers

PSA levels will be assessed prior to start of treatment and on Day 1 of every cycle (every 4 weeks±3 days). The same laboratory should be used for repeat assessments.

Evolution of tumor marker levels will be followed throughout treatment.

### Tumor Tissue Sample Assessment

PTEN analysis will be performed in the baseline biopsy (preferably fresh; archived sample collected within 2 years is acceptable conditional on the biopsy being collected after establishment of castration resistance) by IHC (local or central) or next generation sequencing, to identify patients with PTEN wild-type status.

### Example 3

Following the clinical trial protocol of Example 2, mCRPC patients with histologically confirmed prostate adenocarcinoma were enrolled. Patients who were progressing on either abiraterone acetate or enzalutamide were continued on their backbone AR axis-targeting agents after study enrollment and received treatment with zenocutuzumab in combination. Sites of metastatic involvement included lymph nodes, bone, and/or visceral organs. PTEN status of tumors was established in patients by IHC when sufficient biological material was available, using Roche Ventana Optiview kit DAB and SP218 PTEN antibody using Benchmark Ultra immunohistochemical automated slide stainer. The OptiView DAB IHC Detection Kit (OptiView) is an indirect, biotin-free system for detecting mouse IgG, mouse IgM and rabbit primary antibodies. The kit is intended to identify targets by IHC in sections of formalin-fixed, paraffin-embedded and frozen tissue that are stained on the VENTANA automated slide stainers and visualized by light microscopy. The clinical interpretation of any staining, or the absence of staining, is complemented by morphological studies and evaluation of proper controls.

Among a cohort of ten patients treated, treatment-emergent adverse events (TEAE) related to study treatment classified as a Grade 3 TEAE or higher were not observed. Furthermore, the combination of Zenocutuzumab and abiraterone acetate or enzalutamide was very well tolerated and no safety concerns were noted. Also, no treatment discontinuation due to an adverse event occurred nor were any unexpected signals of toxicity reported. Only a single IRR was reported (i.e. Grade 2, related).

### Example 4

Following the clinical trial protocol of Example 2, a 85 year-old male with mCRPC was treated with a combination of zenocutuzumab and enzalutamide. The patient had received a multitude of prior lines of systemic therapy, including two lines of androgen receptor axis-targeting agents (i.e. abiraterone and enzalutamide) and two lines of chemotherapy (e.g. docetaxel and cabazitaxel) but showed progressive disease after these treatments. Blood-based next-generation sequencing revealed non-sense mutations in NF1 (L1201*) and ERRFI1 (Y403*) and a missense mutation in TP53 (V172D). Per investigator report, the patient had slow radiographic progression in lymph nodes (LN) (target lesions) prior to study enrollment. On study treatment, the patient had a best overall response of stable disease by RECIST (6 months) and no evidence of disease (NED) by bone scans by PCWG3 (6 months), overall stable. After six months of on-going treatment, no evidence of lymph node progression, consistent with radiographic disease control, was reported.

### Example 5

Following the clinical trial protocol of Example 2, a 77 year-old male with mCRPC was treated with a combination of zenocutuzumab and abiraterone acetate/prednisone. The patient had received four prior lines of systemic therapy, including one line of chemotherapy and two lines of androgen receptor axis-targeting agents (i.e. darolutamide and abiraterone) but showed progressive disease after these treatments. PTEN loss was observed by IHC testing performed on archival prostate biopsy sample. No tumor-associated somatic alterations or mutations were detected by blood based next-generation sequencing testing. Per investigator report, the patient had LN progression (non-targets) and metastasis in bone tissue prior to enrollment. On study treatment, the patient had a best overall response of stable disease by RECIST (4 months) and stable disease by bone scans by PCWG3 (4 months), overall stable. After five months of on-going treatment, no evidence of lymph node or bone progression, consistent with radiographic disease control, was reported.

### Example 6

Following the clinical trial protocol of Example 2, a 77 year-old male with mCRPC was treated with a combination of zenocutuzumab and enzalutamide. The patient had received multiple prior lines of systemic therapy, including chemotherapy, a radiopharmaceutical (Xofigo®) and androgen receptor axis-targeting agents, such as docetaxel, abiraterone (in combination with prednisone) and enzalutamide but showed progressive disease after these treatments. The patient's tumor was shown to be PTEN wildtype by IHC testing performed on an archival tumor sample from a prior prostate biopsy. Only a missense mutation was detected in TP53 (A161T) and a nonsense mutation in APC (K139*) by blood based next-generation sequencing testing. On study treatment, the patient had a best overall response of stable disease by measurement of their target lesions (lymph nodes) under RECIST v1.1. per investigator. In the context of a skeletal-related event, the patient was discontinued from treatment.

### Example 7

Following the clinical trial protocol of Example 2, two more patients that were wildtype for PTEN status as established using IHC testing were treated with a combination of zenocutuzumab and enzalutamide. By PCWG3 criteria, PSA values will be assessed for a minimum of 12 weeks after treatment initiation to trend for improvement. Imaging studies will be performed at pre-specified timepoints in the same timeframe to assess for radiographic disease control. These patients had received prior treatment with multiple prior lines of systemic therapy, including chemotherapy and androgen receptor axis-targeting agents, such as abiraterone, docetaxel combined with bicalutamide; cabazitaxel or prednisone combined with enzalutamide but both showed progressive disease after these treatments.

1. (canceled)

2. A method of treating a human subject having a castration-resistant prostate cancer, the method comprising administering to the subject a therapeutically effective amount of a bispecific antibody that comprises an antigen binding site that can bind an extracellular part of ERBB2 and an antigen binding site that can bind an extracellular part of ERBB3, wherein the antigen binding site that binds ErbB2 comprises the CDR1, CDR2 and CDR3 sequences of MF3958 according to SEQ ID NO: 12, wherein the antigen binding site that binds ErbB3 comprises the CDR1, CDR2 and CDR3 sequences of MF3178 according to SEQ ID NO: 47, and wherein both antigen binding sites comprises the CDR1, CDR2 and CDR3 sequences of the light chain variable region according to SEQ ID NO: 161.

3. The method according to claim 2, wherein the cancer has progressed after prior treatment with an androgen receptor axis-targeting agent.

4. The method according to claim 3, wherein the androgen receptor axis-targeting agent is an androgen receptor antagonist.

5. The method according to claim 4, wherein the androgen receptor antagonist is enzalutamide.

6. The method according to claim 3, wherein the androgen receptor axis-targeting agent is an androgen synthesis inhibitor.

7. The method according to claim 3, further comprising administration of an androgen receptor axis-targeting agent selected from enzalutamide and abiraterone acetate to said subject.

8.-11. (canceled)

12. The method according to claim 7, wherein the androgen receptor antagonist used in the prior treatment and the androgen receptor axis-targeting agent further used in the method of treatment with the bispecific antibody, are both enzalutamide.

13. The method according to claim 12, wherein enzalutamide is administered at 160 mg once per day.

14. The method according to claim 2, wherein the cancer has progressed after prior treatment with an androgen synthesis inhibitor and wherein the method further comprises administration of an androgen synthesis inhibitor to said subject.

15. (canceled)

16. The method according to claim 14, wherein the androgen synthesis inhibitor used in the prior treatment and the androgen synthesis inhibitor further used in the method of treatment with the bispecific antibody, are both abiraterone acetate.

17. The method according to claim 7, wherein the abiraterone acetate is administered at 1000 mg once per day.

18. The according to claim 17, wherein the abiraterone acetate is administered in combination with prednisone at 5 mg twice per day.

19. The method according to claim 2, wherein the the bispecific antibody is administered in an amount of 750 mg once every two weeks.

20. The method according to claim 2, wherein the cancer is not NRG1 fusion positive.

21. (canceled)

22. The method according to claim 2, wherein the cancer is a histologically confirmed adenocarcinoma of the prostate without neuroendocrine differentiation or small cell features.

23. (canceled)

24. (canceled)

25. The method according to claim 2, wherein the bispecific antibody comprises
i) the ERBB2 specific heavy chain variable region sequence MF3958, or a heavy chain variable region sequence that differs in at most 15 amino acids from the heavy chain variable region sequence of MF3958 and
ii) the ERBB3 specific heavy chain variable region sequence of MF3178 or a heavy chain variable region sequence that differs in at most 15 amino acids from the heavy chain variable region sequence of MF3178 and
wherein the amino acid differences are not present in the CDR1, CDR2 and CDR3 regions of MF3958 and MF3178.

26. The method according to claim 25, wherein the bispecific antibody comprises the heavy chain variable regions sequences of MF3958 according to SEQ ID NO:12 and MF3178 according to SEQ ID NO: 47.

27. (canceled)

28. The method according to claim 2, wherein the subject or cancer has a phosphatase and tensin homolog (PTEN) wildtype status and wherein PTEN status is determined using immunohistochemistry (IHC).

29. The method according to claim 2, wherein the cancer is metastatic castration-resistant prostate cancer or wherein the subject is at risk of developing metastatic castration-resistant prostate cancer.

30. A kit-of-parts comprising a bispecific antibody that comprises an antigen binding site that can bind an extracellular part of ERBB2 and an antigen binding site that can bind an extracellular part of ERBB3 and an androgen receptor axis-targeting agent selected from enzalutamide and abiraterone acetate, wherein the antigen binding site that binds ErbB2 comprises the CDR1, CDR2 and CDR3 sequences of MF3958 according to SEQ ID NO: 12, wherein the antigen binding site that binds ErbB3 comprises the CDR1, CDR2 and CDR3 sequences of MF3178 according to SEQ ID NO: 47, and wherein both antigen binding sites comprise the CDR1, CDR2 and CDR3 sequences of the light chain variable region according to SEQ ID NO: 161.

31.-36. (canceled)

37. The method according to claim 2, further comprising administration of an androgen receptor axis-targeting agent selected from enzalutamide and abiraterone acetate.

38. The bispecific antibody according to claim 2, wherein the bispecific antibody comprises an antigen binding site that can bind an extracellular part of ERBB2 comprising a CDR1 sequence AYYIN, a CDR2 sequence RIYPGSGYTSYAQKFQG, and a CDR3 sequence PPVYYDSAWFAY; and
ii) an antigen binding site that can bind an extracellular part of ERBB3 comprising a CDR1 sequence GYYMH, a CDR2 sequence WINPNSGGTNYAQKFQG, and a CDR3 sequence DHGSRHFWSYWGFDY, whereby the CDR sequences are according to the Kabat numbering system.

39. The method according to claim 26, wherein both antigen binding sites comprise a variable light chain region with the amino acid sequence according to SEQ ID NO: 161.

40. The method according to claim 39, wherein the bispecific antibody is of the human IgG1 subclass.


SEQUENCE LISTING (183 sequences)

SEQ ID NO: 1 - DNA, 385 bp, synthetic construct
  source 1..385: mol_type = other DNA
ggcccagccggccatggcccaggtgcagctgaagcagtctggggctgagctggtgaggcctggggcttcagtgaagttgtcctgcaaggcttctggctacattttcactggctactatataaactggttgaggcagaggcctggacagggacttgaatggattgcaaaaatttatcctggaagtggtaatacttactacaatgagaagttcaggggcaaggccacactgactgcagaagaatcctccagcactgcctacatgcagctcagcagcctgacatctgaggactctgctgtctatttctgtgcaagagggccccactatgattacgacggcccctggtttgtttactggggccaagggactctggtcaccgtctccagt

SEQ ID NO: 2 - protein, 122 aa, synthetic construct
QVQLKQSGAELVRPGASVKLSCKASGYIFTGYYINWLRQRPGQGLEWIAKIYPGSGNTYYNEKFRGKATLTAEESSSTAYMQLSSLTSEDSAVYFCARGPHYDYDGPWFVYWGQGTLVTVSS

SEQ ID NO: 3 - protein, 5 aa, synthetic construct
GYYIN

SEQ ID NO: 4 - protein, 17 aa, synthetic construct
KIYPGSGNTYYNEKFRG

SEQ ID NO: 5 - protein, 13 aa, synthetic construct
GPHYDYDGPWFVY

SEQ ID NO: 6 - DNA, 382 bp, synthetic construct
  source 1..382: mol_type = other DNA
ggcccagccggccatggcccaggtgcagctgaagcagtctggggctgagctggtgaggcctggggcttcagtgaagctgtcctgcaaggcttctggctacactttcactggctactatataaactgggtgaagcagaggcctggacagggacttgagtggattgcaaggatttatcctggaagtggttatacttactacaatgagaagttcaagggcaaggccacactgactgcagaagaatcctccagcactgcctacatgcacctcagcagcctgacatctgaggactctgctgtctatttctgtgcaagaccccactatggttacgacgactggtacttcggtgtctggggcacaggcaccacggtcaccgtctccagt

SEQ ID NO: 7 - protein, 121 aa, synthetic construct
QVQLKQSGAELVRPGASVKLSCKASGYTFTGYYINWVKQRPGQGLEWIARIYPGSGYTYYNEKFKGKATLTAEESSSTAYMHLSSLTSEDSAVYFCARPHYGYDDWYFGVWGTGTTVTVSS

SEQ ID NO: 8 - protein, 5 aa, synthetic construct
GYYIN

SEQ ID NO: 9 - protein, 17 aa, synthetic construct
RIYPGSGYTYYNEKFKG

SEQ ID NO: 10 - protein, 12 aa, synthetic construct
PHYGYDDWYFGV

SEQ ID NO: 11 - DNA, 382 bp, synthetic construct
  source 1..382: mol_type = other DNA
ggcccagccggccatggcccaggtgcagctggtgcagtctggcgccgaagtgaagaaacctggcgccagcgtgaagctgagctgcaaggccagcggctacaccttcaccgcctactacatcaactgggtccgacaggccccaggccagggcctggaatggatcggcagaatctaccccggctccggctacaccagctacgcccagaagttccagggcagagccaccctgaccgccgacgagagcaccagcaccgcctacatggaactgagcagcctgcggagcgaggataccgccgtgtacttctgcgccagaccccccgtgtactacgacagcgcttggtttgcctactggggccagggcaccctggtcaccgtctccagt

SEQ ID NO: 12 - protein, 121 aa, synthetic construct
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPPVYYDSAWFAYWGQGTLVTVSS

SEQ ID NO: 13 - protein, 5 aa, synthetic construct
AYYIN

SEQ ID NO: 14 - protein, 17 aa, synthetic construct
RIYPGSGYTSYAQKFQG

SEQ ID NO: 15 - protein, 12 aa, synthetic construct
PPVYYDSAWFAY

SEQ ID NO: 16 - DNA, 382 bp, synthetic construct
  source 1..382: mol_type = other DNA
ggcccagccggccatggcccaggtgcagctgaagcagtctggggctgagctggtgaggcctggggcttcagtgaaactgtcctgcaaggcttctggctacactttcactgcctactatataaactgggtgaagcagaggcctggacagggacttgagtggattgcaaggatttatcctggaagtggctatacttactacaatgagattttcaagggcagggccacactgactgcagacgaatcctccagcactgcctacatgcaactcagcagcctgacatctgaggactctgctgtctatttctgtgcaagacctccggtctactatgactcggcctggtttgcttactggggccaagggactctggtcaccgtctccagt

SEQ ID NO: 17 - protein, 121 aa, synthetic construct
QVQLKQSGAELVRPGASVKLSCKASGYTFTAYYINWVKQRPGQGLEWIARIYPGSGYTYYNEIFKGRATLTADESSSTAYMQLSSLTSEDSAVYFCARPPVYYDSAWFAYWGQGTLVTVSS

SEQ ID NO: 18 - protein, 5 aa, synthetic construct
AYYIN

SEQ ID NO: 19 - protein, 18 aa, synthetic construct
RIYPGSGSYTYYNEIFKG

SEQ ID NO: 20 - protein, 12 aa, synthetic construct
PPVYYDSAWFAY

SEQ ID NO: 21 - DNA, 382 bp, synthetic construct
  source 1..382: mol_type = other DNA
ggcccagccggccatggcccaggtgcagctgaagcagtctggggctgagctggtgaggcctgggacttcagtgaagctgtcctgcaaggcttctggctacactttcactggctactatataaactgggtgaagcagaggcctggacagggacttgagtggattgcaaggatttatcctggaagtggttatacttactacaatgagaagttcaagggcaaggccacactgactgcagaagaatcctccaacactgcctatatgcacctcagcagcctgacatctgaggactctgctgtctatttctgtgcaaggccccactatggttacgacgactggtacttcgctgtctggggcacagggaccacggtcaccgtctccagt

SEQ ID NO: 22 - protein, 121 aa, synthetic construct
QVQLKQSGAELVRPGTSVKLSCKASGYTFTGYYINWVKQRPGQGLEWIARIYPGSGYTYYNEKFKGKATLTAEESSNTAYMHLSSLTSEDSAVYFCARPHYGYDDWYFAVWGTGTTVTVSS

SEQ ID NO: 23 - protein, 5 aa, synthetic construct
GYYIN

SEQ ID NO: 24 - protein, 17 aa, synthetic construct
RIYPGSGYTYYNEKFKG

SEQ ID NO: 25 - protein, 12 aa, synthetic construct
PHYGYDDWYFAV

SEQ ID NO: 26 - DNA, 385 bp, synthetic construct
  source 1..385: mol_type = other DNA
ggcccagccggccatggcccaggtccagctgcagcagtctggggctgagctggtgaggcctggggcttcagtgaagctgtcctgcaaggcttctggctacactttcactggctactatataaactgggtgaagcagaggcctggacagggacttgagtggattgcaaggatttatcctggcagtggtcatacttcctacaatgagaagttcaagggcaaggccacactgactacagaaaaatcctccagcactgcctacatgcagctcagcagcctgacatctgaggactctgctgtctatttctgtgcaagacctatctactttgattacgcaggggggtacttcgatgtctggggcacaagaacctcggtcaccgtctccagt

SEQ ID NO: 27 - protein, 122 aa, synthetic construct
QVQLQQSGAELVRPGASVKLSCKASGYTFTGYYINWVKQRPGQGLEWIARIYPGSGHTSYNEKFKGKATLTTEKSSSTAYMQLSSLTSEDSAVYFCARPIYFDYAGGYFDVWGTRTSVTVSS

SEQ ID NO: 28 - protein, 5 aa, synthetic construct
GYYIN

SEQ ID NO: 29 - protein, 17 aa, synthetic construct
RIYPGSGHTSYNEKFKG

SEQ ID NO: 30 - protein, 13 aa, synthetic construct
PIYFDYAGGYFDV

SEQ ID NO: 31 - DNA, 382 bp, synthetic construct
  source 1..382: mol_type = other DNA
ggcccagccggccatggcccaggtgcagctggtgcagtctggcgccgaagtgaagaaacctggcgccagcgtgaagctgagctgcaaggccagcggctacaccttcaccgcctactacatcaactgggtccgacaggccccaggccagggcctggaatggatcggcagaatctaccccggctccggctacaccagctacgcccagaagttccagggcagagccaccctgaccgccgacgagagcaccagcaccgcctacatggaactgagcagcctgcggagcgaggataccgccgtgtacttctgcgccagaccccactacggctacgacgactggtacttcggcgtgtggggccagggcaccctggtcaccgtctccagt

SEQ ID NO: 32 - protein, 121 aa, synthetic construct
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPHYGYDDWYFGVWGQGTLVTVSS

SEQ ID NO: 33 - protein, 5 aa, synthetic construct
AYYIN

SEQ ID NO: 34 - protein, 17 aa, synthetic construct
RIYPGSGYTSYAQKFQG

SEQ ID NO: 35 - protein, 12 aa, synthetic construct
PHYGYDDWYFGV

SEQ ID NO: 36 - DNA, 382 bp, synthetic construct
  source 1..382: mol_type = other DNA
ggcccagccggccatggcccaggtccagctgcagcagtctggggctgagctggtgaggcctggggcttcagtgaagctgtcctgcaaggcttctggctacactttcactgcctactatataaactgggtgaagcagaggcctggacagggacttgagtggattgcaaagatttatcctggaagtggttatacttactacaatgagaatttcaggggcaaggccacactgactgcagaagaatcctccagtactgcctacatacaactcagcagcctgacatctgaggactctgctgtctatttctgtgcaagaggcgtctatgattacgacggggcctggtttgcttactggggccaagggactctggtcaccgtctccagt

SEQ ID NO: 37 - protein, 121 aa, synthetic construct
QVQLQQSGAELVRPGASVKLSCKASGYTFTAYYINWVKQRPGQGLEWIAKIYPGSGYTYYNENFRGKATLTAEESSSTAYIQLSSLTSEDSAVYFCARGVYDYDGAWFAYWGQGTLVTVSS

SEQ ID NO: 38 - protein, 5 aa, synthetic construct
AYYIN

SEQ ID NO: 39 - protein, 17 aa, synthetic construct
KIYPGSGYTYYNENFRG

SEQ ID NO: 40 - protein, 12 aa, synthetic construct
GVYDYDGAWFAY

SEQ ID NO: 41 - DNA, 379 bp, synthetic construct
  source 1..379: mol_type = other DNA
ggcccagccggccatggcccaggtgcagctgaagcagtctggacctgagctggtgaagcctggggcctcagtgaagatttcctgcaaggcttctggcgacgcattcagttactcctggatgaactgggtgaagcagaggcctggaaagggtcttgagtggattggacggatttatcctggagatggagatattaactacaatgggaagttcaagggcaaggccacactgactgcagacaaatcctccagcacagcccacctgcaactcaacagcctgacatctgaggactctgcggtctacttctgtgcaagaggacagctcggactagaggcctggtttgcttattggggccaggggactctggtcaccgtctccagt

SEQ ID NO: 42 - protein, 120 aa, synthetic construct
QVQLKQSGPELVKPGASVKISCKASGDAFSYSWMNWVKQRPGKGLEWIGRIYPGDGDINYNGKFKGKATLTADKSSSTAHLQLNSLTSEDSAVYFCARGQLGLEAWFAYWGQGTLVTVSS

SEQ ID NO: 43 - protein, 5 aa, synthetic construct
YSWMN

SEQ ID NO: 44 - protein, 17 aa, synthetic construct
RIYPGDGDINYNGKFKG

SEQ ID NO: 45 - protein, 11 aa, synthetic construct
GQLGLEAWFAY

SEQ ID NO: 46 - DNA, 391 bp, synthetic construct
  source 1..391: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 47 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 48 - protein, 5 aa, synthetic construct
GYYMH

SEQ ID NO: 49 - protein, 17 aa, synthetic construct
WINPNSGGTNYAQKFQG

SEQ ID NO: 50 - protein, 15 aa, synthetic construct
DHGSRHFWSYWGFDY

SEQ ID NO: 51 - DNA, 385 bp, synthetic construct
  source 1..385: mol_type = other DNA
ggcccagccggccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggctgtgtattactgtgcaagagattggtggtacccgccgtactactggggctttgattattggggccaaggtaccctggtcaccgtctccagt

SEQ ID NO: 52 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWWYPPYYWGFDYWGQGTLVTVSS

SEQ ID NO: 53 - protein, 5 aa, synthetic construct
SYAMS

SEQ ID NO: 54 - protein, 17 aa, synthetic construct
AISGSGGSTYYADSVKG

SEQ ID NO: 55 - protein, 13 aa, synthetic construct
DWWYPPYYWGFDY

SEQ ID NO: 56 - DNA, 391 bp, synthetic construct
  source 1..391: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggccgtgtattactgtgcaaaagattcttactctcgtcatttctactcttggtgggcctttgattattggggccaaggtaccctggtcaccgtctccagt

SEQ ID NO: 57 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAKDSYSRHFYSWWAFDYWGQGTLVTVSS

SEQ ID NO: 58 - protein, 5 aa, synthetic construct
GYYMH

SEQ ID NO: 59 - protein, 17 aa, synthetic construct
WINPNSGGTNYAQKFQG

SEQ ID NO: 60 - protein, 15 aa, synthetic construct
DSYSRHFYSWWAFDY

SEQ ID NO: 61 - DNA, 382 bp, synthetic construct
  source 1..382: mol_type = other DNA
ggcccagccggccatggcccaggtgcagctggtgcagtctggggctgacgtgaagaagcctggggcctcagtgaaggtctcctgcaaggcttctggatacaccttcaccggctactatatgcactgggtgcgacaggcccctggacaagctcttgagtggatgggatggatcaacccttctagtggtggcacaaactatgcaaagaagtttcagggcagggtcacgatgaccagggagacgtccacaagcacagcctacatggagctgagcaggctgagatctgacgacacggctacgtattactgtgcaagagatcatggttctcgtcatttctggtcttactggggctttgattattggggccaaggtaccctggtcacc

SEQ ID NO: 62 - protein, 124 aa, synthetic construct
QVQLVQSGADVKKPGASVKVSCKASGYTFTGYYMHWVRQAPGQALEWMGWINPSSGGTNYAKKFQGRVTMTRETSTSTAYMELSRLRSDDTATYYCARDHGSRHFWSYWGFDYWGQGTLVTVSS

SEQ ID NO: 63 - protein, 5 aa, synthetic construct
GYYMH

SEQ ID NO: 64 - protein, 17 aa, synthetic construct
WINPSSGGTNYAKKFQG

SEQ ID NO: 65 - protein, 15 aa, synthetic construct
DHGSRHFWSYWGFDY

SEQ ID NO: 66 - DNA, 382 bp, synthetic construct
  source 1..382: mol_type = other DNA
ggcccagccggccatggcccaggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaacccttctagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcacc

SEQ ID NO: 67 - protein, 124 aa, synthetic construct
QVQLVQSGADVKKPGASVKVTCKASGYTFTGYYMHWVRQAPGQALEWMGWINPSSGGTNYAKKFQGRVSMTRETSTSTAYMQLSRLRSDDTATYYCARDHGSRHFWSYWGFDYWGQGTLVTVSS

SEQ ID NO: 68 - protein, 5 aa, synthetic construct
GYYMH

SEQ ID NO: 69 - protein, 17 aa, synthetic construct
WINPSSGGTNYAKKFQG

SEQ ID NO: 70 - protein, 15 aa, synthetic construct
DHGSRHFWSYWGFDY

SEQ ID NO: 71 - DNA, 382 bp, synthetic construct
  source 1..382: mol_type = other DNA
ggcccagccggccatggcccaggtgcagctggtgcagtctggggctgatgtgaagaagcctggggcctcagtgaaggtcacgtgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccatcagcacagcctacatgcagctgagcaggctgagatctgacgacacggctgtgtattactgtgcaagagatcatggttctcgtcatttctggtcttactggggctttgattattggggccaaggtaccctggtcacc

SEQ ID NO: 72 - protein, 124 aa, synthetic construct
QVQLVQSGADVKKPGASVKVTCKASGYTFTGYYMHWVRQAPGQGLEWMGWINPQSGGTNYAQKFQGRVTMTRDTSISTAYMQLSRLRSDDTAVYYCARDHGSRHFWSYWGFDYWGQGTLVTVSS

SEQ ID NO: 73 - protein, 5 aa, synthetic construct
GYYMH

SEQ ID NO: 74 - protein, 17 aa, synthetic construct
WINPQSGGTNYAQKFQG

SEQ ID NO: 75 - protein, 15 aa, synthetic construct
DHGSRHFWSYWGFDY

SEQ ID NO: 76 - DNA, 391 bp, synthetic construct
  source 1..391: mol_type = other DNA
ggcccagccggccatggcccaggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaaccctcaaagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcaccgtctccagt

SEQ ID NO: 77 - protein, 124 aa, synthetic construct
QVQLVQSGADVKKPGASVKVTCKASGYTFTGYYMHWVRQAPGQALEWMGWINPQSGGTNYAKKFQGRVSMTRETSTSTAYMQLSRLRSDDTATYYCARDHGSRHFWSYWGFDYWGQGTLVTVSS

SEQ ID NO: 78 - protein, 5 aa, synthetic construct
GYYMH

SEQ ID NO: 79 - protein, 17 aa, synthetic construct
WINPQSGGTNYAKKFQG

SEQ ID NO: 80 - protein, 15 aa, synthetic construct
DHGSRHFWSYWGFDY

SEQ ID NO: 81 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctggcagtggttctacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 82 - protein, 124 aa, synthetic construct
QVQLVQSGAEVKKPGASVKVSCKASGYTFTGYYMHWVRQAPGQGLEWMGWINPGSGSTNYAQKFQGRVTMTRDTSISTAYMELSRLRSDDTAVYYCARDHGSRHFWSYWGFDYWGQGTLVTVSS

SEQ ID NO: 83 - protein, 5 aa, synthetic construct
GYYMH

SEQ ID NO: 84 - protein, 17 aa, synthetic construct
WINPGSGSTNYAQKFQG

SEQ ID NO: 85 - protein, 15 aa, synthetic construct
DHGSRHFWSYWGFDY

SEQ ID NO: 86 - DNA, 382 bp, synthetic construct
  source 1..382: mol_type = other DNA
ggcccagccggccatggcccaggtgcagctggtgcagtctggggctgacgtgaagaagcctggggcctcagtgaaggtctcctgcaaggcttctggatacaccttcaccggctactatatgcactgggtgcgacaggcccctggacaagctcttgagtggatgggatggatcaaccctcaaagtggtggcacaaactatgcaaagaagtttcagggcagggtcacgatgaccagggagacgtccacaagcacagcctacatggagctgagcaggctgagatctgacgacacggctacgtattactgtgcaagagatcatggttctcgtcatttctggtcttactggggctttgattattggggccaaggtaccctggtcacc

SEQ ID NO: 87 - protein, 124 aa, synthetic construct
QVQLVQSGADVKKPGASVKVSCKASGYTFTGYYMHWVRQAPGQALEWMGWINPQSGGTNYAKKFQGRVTMTRETSTSTAYMELSRLRSDDTATYYCARDHGSRHFWSYWGFDYWGQGTLVTVSS

SEQ ID NO: 88 - protein, 5 aa, synthetic construct
GYYMH

SEQ ID NO: 89 - protein, 17 aa, synthetic construct
WINPQSGGTNYAKKFQG

SEQ ID NO: 90 - protein, 15 aa, synthetic construct
DHGSRHFWSYWGFDY

SEQ ID NO: 91 - DNA, 391 bp, synthetic construct
  source 1..391: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttaagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 92 - protein, 124 aa, synthetic construct
QVQLVQSGAEVKKPGASVKVSCKASGYTFTGYYMHWVRQAPGQGLEWMGWINPQSGGTNYAQKFKGRVTMTRDTSTSTAYMELSRLRSDDTAVYYCARDHGSRHFWSYWGFDYWGQGTLVTVSS

SEQ ID NO: 93 - protein, 5 aa, synthetic construct
GYYMH

SEQ ID NO: 94 - protein, 17 aa, synthetic construct
WINPQSGGTNYAQKFKG

SEQ ID NO: 95 - protein, 15 aa, synthetic construct
DHGSRHFWSYWGFDY

SEQ ID NO: 96 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctggcagtggttctacaaactatgcacagaagtttcagggcagggtcacgatgaccagggacacgtccacaagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 97 - protein, 124 aa, synthetic construct
QVQLVQSGAEVKKPGASVKVSCKASGYTFTGYYMHWVRQAPGQGLEWMGWINPGSGSTNYAQKFQGRVTMTRDTSTSTAYMELSRLRSDDTAVYYCARDHGSRHFWSYWGFDYWGQGTLVTVSS

SEQ ID NO: 98 - protein, 5 aa, synthetic construct
GYYMH

SEQ ID NO: 99 - protein, 17 aa, synthetic construct
WINPGSGSTNYAQKFQG

SEQ ID NO: 100 - protein, 15 aa, synthetic construct
DHGSRHFWSYWGFDY

SEQ ID NO: 101 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcaaagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 102 - protein, 124 aa, synthetic construct
QVQLVQSGAEVKKPGASVKVSCKASGYTFTGYYMHWVRQAPGQGLEWMGWINPQSGGTNYAKKFQGRVTMTRDTSTSTAYMELSRLRSDDTAVYYCARDHGSRHFWSYWGFDYWGQGTLVTVSS

SEQ ID NO: 103 - protein, 5 aa, synthetic construct
GYYMH

SEQ ID NO: 104 - protein, 17 aa, synthetic construct
WINPQSGGTNYAKKFQG

SEQ ID NO: 105 - protein, 15 aa, synthetic construct
DHGSRHFWSYWGFDY

SEQ ID NO: 106 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggaaaggggcttgagtggatgggatggatcaaccctcagagtggtggcacaaactatgcacagaagtttcagggcagggtcacgatgaccagggacacgtccacgagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 107 - protein, 124 aa, synthetic construct
QVQLVQSGAEVKKPGASVKVSCKASGYTFTGYYMHWVRQAPGKGLEWMGWINPQSGGTNYAQKFQGRVTMTRDTSTSTAYMELSRLRSDDTAVYYCARDHGSRHFWSYWGFDYWGQGTLVTVSS

SEQ ID NO: 108 - protein, 5 aa, synthetic construct
GYYMH

SEQ ID NO: 109 - protein, 17 aa, synthetic construct
WINPQSGGTNYAQKFQG

SEQ ID NO: 110 - protein, 15 aa, synthetic construct
DHGSRHFWSYWGFDY

SEQ ID NO: 111 - DNA, 391 bp, synthetic construct
  source 1..391: mol_type = other DNA
ggcccagccggccatggcccaggtgcagctggtgcagtctggggctgaggtgaagaagcctggggcctcagtgaaggtctcctgcaaggcttctggatacaccttcacctcttactatatgcactgggtgcgacaggcccctggacaagggcttgagtggatgggatggatcaaccctcaggggggttctacaaactatgcacagaagtttcagggcagggtcacgatgaccagggacacgtccaccagcacagtgtacatggagctgagcaggctgagatctgaggacacggctgtgtattactgtgcaagagatcatggttctcgtcatttctggtcttactggggctttgattattggggccaaggtaccctggtcaccgtctccagt

SEQ ID NO: 112 - protein, 124 aa, synthetic construct
QVQLVQSGAEVKKPGASVKVSCKASGYTFTSYYMHWVRQAPGQGLEWMGWINPQGGSTNYAQKFQGRVTMTRDTSTSTVYMELSRLRSEDTAVYYCARDHGSRHFWSYWGFDYWGQGTLVTVSS

SEQ ID NO: 113 - protein, 5 aa, synthetic construct
SYYMH

SEQ ID NO: 114 - protein, 17 aa, synthetic construct
WINPQGGSTNYAQKFQG

SEQ ID NO: 115 - protein, 15 aa, synthetic construct
DHGSRHFWSYWGFDY

SEQ ID NO: 116 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggttctacaaactatgcacagaagtttcagggcagggtcacgatgaccagggacacgtccaccagcacagcctacatggagctgagctctctgagatctgaggacacggctgtgtattactgtgcaagagatcatggttctcgtcatttctggtcttactggggctttgattattggggccaaggtaccctggtcacc

SEQ ID NO: 117 - protein, 124 aa, synthetic construct
QVQLVQSGAEVKKPGASVKVSCKASGYTFTGYYMHWVRQAPGQGLEWMGWINPQSGSTNYAQKFQGRVTMTRDTSTSTAYMELSSLRSEDTAVYYCARDHGSRHFWSYWGFDYWGQGTLVTVSS

SEQ ID NO: 118 - protein, 5 aa, synthetic construct
GYYMH

SEQ ID NO: 119 - protein, 17 aa, synthetic construct
WINPQSGSTNYAQKFQG

SEQ ID NO: 120 - protein, 15 aa, synthetic construct
DHGSRHFWSYWGFDY

SEQ ID NO: 121 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccaccagcacagtctacatggagctgagctctctgagatctgacgacacggctgtgtattactgtgcaagagatcatggttctcgtcatttctggtcttactggggctttgattattggggccaaggtaccctggtcacc

SEQ ID NO: 122 - protein, 124 aa, synthetic construct
QVQLVQSGAEVKKPGASVKVSCKASGYTFTGYYMHWVRQAPGQGLEWMGWINPQSGGTNYAQKFQGRVTMTRDTSTSTVYMELSSLRSDDTAVYYCARDHGSRHFWSYWGFDYWGQGTLVTVSS

SEQ ID NO: 123 - protein, 5 aa, synthetic construct
GYYMH

SEQ ID NO: 124 - protein, 17 aa, synthetic construct
WINPQSGGTNYAQKFQG

SEQ ID NO: 125 - protein, 15 aa, synthetic construct
DHGSRHFWSYWGFDY

SEQ ID NO: 126 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 127 - protein, 124 aa, synthetic construct
QVQLVQSGAEVKKPGASVKVSCKASGYTFTGYYMHWVRQAPGQGLEWMGWINPQSGGTNYAQKFQGRVTMTRDTSTSTAYMELSRLRSDDTAVYYCARDHGSRHFWSYWGFDYWGQGTLVTVSS

SEQ ID NO: 128 - protein, 5 aa, synthetic construct
GYYMH

SEQ ID NO: 129 - protein, 17 aa, synthetic construct
WINPQSGGTNYAQKFQG

SEQ ID NO: 130 - protein, 15 aa, synthetic construct
DHGSRHFWSYWGFDY

SEQ ID NO: 131 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 132 - protein, 124 aa, synthetic construct
QVQLVQSGAEVKKPGASVKVSCKASGYTFTGYYMHWVRQAPGQGLEWMGWINPQSGGTNYAQKFQGRVTMTRDTSISTAYMELSRLRSDDTAVYYCARDHGSRHFWSYWGFDYWGQGTLVTVSS

SEQ ID NO: 133 - protein, 5 aa, synthetic construct
GYYMH

SEQ ID NO: 134 - protein, 17 aa, synthetic construct
WINPQSGGTNYAQKFQG

SEQ ID NO: 135 - protein, 15 aa, synthetic construct
DHGSRHFWSYWGFDY

SEQ ID NO: 136 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcacctcttactatatgcactgggtgcgacaggcccctggacaagggcttgagtggatgggatggatcaacccttctgggggttctacaaactatgcacagaagtttcagggcagggtcacgatgaccagggacacgtccaccagcacagtgtacatggagctgagcaggctgagatctgaggacacggctgtgtattactgtgcaagagatcatggttctcgtcatttctggtcttactggggctttgattattggggccaaggtaccctggtcacc

SEQ ID NO: 137 - protein, 124 aa, synthetic construct
QVQLVQSGAEVKKPGASVKVSCKASGYTFTSYYMHWVRQAPGQGLEWMGWINPSGGSTNYAQKFQGRVTMTRDTSTSTVYMELSRLRSEDTAVYYCARDHGSRHFWSYWGFDYWGQGTLVTVSS

SEQ ID NO: 138 - protein, 5 aa, synthetic construct
SYYMH

SEQ ID NO: 139 - protein, 17 aa, synthetic construct
WINPSGGSTNYAQKFQG

SEQ ID NO: 140 - protein, 15 aa, synthetic construct
DHGSRHFWSYWGFDY

SEQ ID NO: 141 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggttctacaaactatgcacagaagtttcagggcagggtcacgatgaccagggacacgtccaccagcacagcctacatggagctgagctctctgagatctgaggacacggctgtgtattactgtgcaagagatcatggttctcgtcatttctggtcttactggggctttgattattggggccaaggtaccctggtcacc

SEQ ID NO: 142 - protein, 124 aa, synthetic construct
QVQLVQSGAEVKKPGASVKVSCKASGYTFTGYYMHWVRQAPGQGLEWMGWINPSSGSTNYAQKFQGRVTMTRDTSTSTAYMELSSLRSEDTAVYYCARDHGSRHFWSYWGFDYWGQGTLVTVSS

SEQ ID NO: 143 - protein, 5 aa, synthetic construct
GYYMH

SEQ ID NO: 144 - protein, 17 aa, synthetic construct
WINPSSGSTNYAQKFQG

SEQ ID NO: 145 - protein, 15 aa, synthetic construct
DHGSRHFWSYWGFDY

SEQ ID NO: 146 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccaccagcacagtctacatggagctgagctctctgagatctgacgacacggctgtgtattactgtgcaagagatcatggttctcgtcatttctggtcttactggggctttgattattggggccaaggtaccctggtcacc

SEQ ID NO: 147 - protein, 124 aa, synthetic construct
QVQLVQSGAEVKKPGASVKVSCKASGYTFTGYYMHWVRQAPGQGLEWMGWINPSSGGTNYAQKFQGRVTMTRDTSTSTVYMELSSLRSDDTAVYYCARDHGSRHFWSYWGFDYWGQGTLVTVSS

SEQ ID NO: 148 - protein, 5 aa, synthetic construct
GYYMH

SEQ ID NO: 149 - protein, 17 aa, synthetic construct
WINPSSGGTNYAQKFQG

SEQ ID NO: 150 - protein, 15 aa, synthetic construct
DHGSRHFWSYWGFDY

SEQ ID NO: 151 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 152 - protein, 124 aa, synthetic construct
QVQLVQSGAEVKKPGASVKVSCKASGYTFTGYYMHWVRQAPGQGLEWMGWINPSSGGTNYAQKFQGRVTMTRDTSTSTAYMELSRLRSDDTAVYYCARDHGSRHFWSYWGFDYWGQGTLVTVSS

SEQ ID NO: 153 - protein, 5 aa, synthetic construct
GYYMH

SEQ ID NO: 154 - protein, 17 aa, synthetic construct
WINPSSGGTNYAQKFQG

SEQ ID NO: 155 - protein, 15 aa, synthetic construct
DHGSRHFWSYWGFDY

SEQ ID NO: 156 - DNA, 382 bp, synthetic construct
  source 1..382: mol_type = other DNA
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 157 - protein, 124 aa, synthetic construct
QVQLVQSGAEVKKPGASVKVSCKASGYTFTGYYMHWVRQAPGQGLEWMGWINPSSGGTNYAQKFQGRVTMTRDTSISTAYMELSRLRSDDTAVYYCARDHGSRHFWSYWGFDYWGQGTLVTVSS

SEQ ID NO: 158 - protein, 5 aa, synthetic construct
GYYMH

SEQ ID NO: 159 - protein, 17 aa, synthetic construct
WINPSSGGTNYAQKFQG

SEQ ID NO: 160 - protein, 15 aa, synthetic construct
DHGSRHFWSYWGFDY

SEQ ID NO: 161 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 162 - protein, 107 aa, Homo sapiens [sequence omitted]

SEQ ID NO: 163 - DNA, 321 bp, Homo sapiens
  source 1..321: mol_type = other DNA
  CDS 1..321: protein_id = 164; translation = DIQMTQSPSSLSASVGDRVTITCRASQSISSYLNWYQQKPGKAP KLLIYAASSLQSGVPSRFSGSGSGTDFTLTISSLQPEDFATYYCQQSYSTPPTFGQGT KVEIK
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 164 - protein, 107 aa, Homo sapiens; identical to SEQ ID NO: 161

SEQ ID NO: 165 - DNA, 324 bp, Homo sapiens
  source 1..324: mol_type = genomic DNA
  CDS 1..324: protein_id = 166
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 166 - protein, 107 aa, Homo sapiens [sequence omitted]

SEQ ID NO: 167 - protein, 107 aa, Homo sapiens; identical to SEQ ID NO: 161

SEQ ID NO: 168 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 169 - protein, 6 aa, synthetic construct
QSISSY

SEQ ID NO: 170 - skipped

SEQ ID NO: 171 - protein, 9 aa, synthetic construct
QQSYSTPPT

SEQ ID NO: 172 - DNA, 294 bp, synthetic construct
  source 1..294: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 173 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 174 - DNA, 45 bp, synthetic construct
  source 1..45: mol_type = other DNA
gagcccaaatcttgtgacaaaactcacacatgcccaccgtgccca

SEQ ID NO: 175 - protein, 15 aa, synthetic construct
EPKSCDKTHTCPPCP

SEQ ID NO: 176 - DNA, 330 bp, synthetic construct
  source 1..330: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 177 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 178 - DNA, 321 bp, synthetic construct
  source 1..321: mol_type = other DNA
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 179 - protein, 106 aa, synthetic construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - DNA, 321 bp, synthetic construct
  source 1..321: mol_type = other DNA
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 181 - protein, 106 aa, synthetic construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 11 aa, synthetic construct
RASQSISSYLN

SEQ ID NO: 183 - protein, 7 aa, synthetic construct
AASSLQS